SEQUENCE LISTING (56 sequences)

SEQ ID NO: 1 - protein, 23 aa, Artificial Sequence
  M2 ectodomain peptide #1 (M2e#1_C)
SLLTEVETPTRNEWECRCSDSSD

SEQ ID NO: 2 - protein, 23 aa, Artificial Sequence
  M2 ectodomain peptide #2 (M2e#2_C)
SLLTEVETPTRNEWECKCIDSSD

SEQ ID NO: 3 - protein, 23 aa, Artificial Sequence
  M2 ectodomain peptide #3 (M2e#3_C)
SLLTEVETPIRNEWGCRCNGSSD

SEQ ID NO: 4 - protein, 23 aa, Artificial Sequence
  M2 ectodomain peptide #4 (M2e#4_C)
SLLTEVETPIRNEWGCRCNDSSD

SEQ ID NO: 5 - protein, 23 aa, Artificial Sequence
  M2 ectodomain peptide #5 (M2e#5_C)
SLLTEVETLTRNGWECRCSDSSD

SEQ ID NO: 6 - protein, 23 aa, Artificial Sequence
  M2 ectodomain #6 (M2e#6_C)
SLLTEVETPTRNGWECKCSDSSD

SEQ ID NO: 7 - protein, 23 aa, Artificial Sequence
  M2 ectodomain #1 with serine substitutions (M2e#1_S)
SLLTEVETPTRNEWESRSSDSSD

SEQ ID NO: 8 - protein, 23 aa, Artificial Sequence
  M2 ectodomain #2 with serine substitutions (M2e#2_S)
SLLTEVETPTRNEWESKSIDSSD

SEQ ID NO: 9 - protein, 23 aa, Artificial Sequence
  M2 ectodomain #3 with serine substitutions (M2e#3_S)
SLLTEVETPIRNEWGSRSNGSSD

SEQ ID NO: 10 - protein, 23 aa, Artificial Sequence
  M2 ectodomain #4 with serine substitutions (M2e#4_S)
SLLTEVETPIRNEWGSRSNDSSD

SEQ ID NO: 11 - protein, 23 aa, Artificial Sequence
  M2 ectodomain #5 with serine substitutions (M2e#5_S)
SLLTEVETLTRNGWESRSSDSSD

SEQ ID NO: 12 - protein, 23 aa, Artificial Sequence
  M2 ectodomain #6 with serine substitutions (M2e#6_S)
SLLTEVETPTRNGWESKSSDSSD

SEQ ID NO: 13 - DNA, 294 bp, Influenza virus
atgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctctttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 14 - protein, 97 aa, Influenza virus
MSLLTEVETPIRNEWGCRCNGSSDPLTIAANIIGILHLTLWILDRLFFKCIYRRFKYGLKGGPSTEGVPKSMREEYRKEQQSAVDADDGHFVSIELE

SEQ ID NO: 15 - DNA, 546 bp, Artificial Sequence
  METR_C
atgagcctgctgaccgaggtggagacccctaccaggaacgagtgggaatgcaggtgcagcgacagcagcgacggatctgcttcaggaagtctgctcaccgaagtggaaacacctaccagaaatgaatgggagtgtaagtgcatcgatagcagtgactctggttctggagctagcctcctgacagaggttgagactcccatcaggaatgagtggggctgcaggtgtaacggctcttcagattcagctggatctggttcactccttacagaagtggagacaccaatcagaaacgagtggggctgtaggtgcaacgatagtagcgatcaggtgcacttccagcctctgcctcctgccgtggtgaagctgagtctccttaccgaggttgaaaccctgacccggaacggctgggagtgtaggtgtagcgacagcagtgaccagttcatcaaggccaacagcaagttcatcggcatcaccgagtctctgcttactgaagtcgagactccaactaggaacggctgggaatgcaagtgctccgacagttcagat

SEQ ID NO: 16 - protein, 181 aa, Artificial Sequence
  M2e Tandem Repeats_C
SLLTEVETPTRNEWECRCSDSSDGSASGSLLTEVETPTRNEWECKCIDSSDSGSGASLLTEVETPIRNEWGCRCNGSSDSAGSGSLLTEVETPIRNEWGCRCNDSSDQVHFQPLPPAVVKLSLLTEVETLTRNGWECRCSDSSDQFIKANSKFIGITESLLTEVETPTRNGWECKCSDSSD

SEQ ID NO: 17 - DNA, 552 bp, Artificial Sequence
  M2e Tandem Repeats_S
atgagcctgctgaccgaggtggagacccctaccaggaacgagtgggaaagcaggagtagcgacagcagcgacggatctgcttcaggaagtctgctcaccgaagtggaaacacctaccagaaatgaatgggagagtaagagtatcgatagcagtgactctggttctggagctagcctcctgacagaggttgagactcccatcaggaatgagtggggctcaaggagtaacggctcttcagattcagctggatctggttcactccttacagaagtggagacaccaatcagaaacgagtggggctctaggtccaacgatagtagcgatcaggtgcacttccagcctctgcctcctgccgtggtgaagctgagtctccttaccgaggttgaaaccctgacccggaacggctgggagtccaggtcaagcgacagcagtgaccagttcatcaaggccaacagcaagttcatcggcatcaccgagtctctgcttactgaagtcgagactccaactaggaacggctgggaatctaagagttccgacagttcagattagtag

SEQ ID NO: 18 - protein, 181 aa, Artificial Sequence
  M2e Tandem Repeats_S
SLLTEVETPTRNEWESRSSDSSDGSASGSLLTEVETPTRNEWESKSIDSSDSGSGASLLTEVETPIRNEWGSRSNGSSDSAGSGSLLTEVETPIRNEWGSRSNDSSDQVHFQPLPPAVVKLSLLTEVETLTRNGWESRSSDSSDQFIKANSKFIGITESLLTEVETPTRNGWESKSSDSSD

SEQ ID NO: 19 - DNA, 1497 bp, Artificial Sequence
  NP consensus sequence
atggcgtcccaaggcaccaaacggtcatatgaacagatggaaactgatggggatcgccagaatgcaactgagattagggcatccgtcgggaagatgattgatggaattgggagattctacatccaaatgtgcactgaacttaaactcagtgatcatgaagggcggttgatccagaacagcttgacaatagagaaaatggtgctctctgcttttgatgaaagaaggaataaatacctggaagaacaccccagcgcggggaaagatcccaagaaaactggggggcccatatacaggagagtagatggaaaatggatgagggaactcgtcctttatgacaaagaagaaataaggcgaatctggcgccaagccaacaatggtgaggatgctacagctggtctaactcacataatgatttggcattccaatttgaatgatgcaacataccagaggacaagagctcttgttcgaactggaatggatcccagaatgtgctctctgatgcagggctcgaccctccctagaaggtccggagctgcaggtgctgcagtcaaaggaatcgggacaatggtgatggaactgatcagaatggtcaaacgggggatcaacgatcgaaatttttggagaggtgagaatgggcggaaaacaagaagtgcttatgagagaatgtgcaacattcttaaaggaaaatttcaaacagctgcacaaagagcaatggtggatcaagtgagagaaagtcggaacccaggaaacgctgagatcgaagatctcatatttttagcaagatctgcattgatattgagaggatcagttgctcacaaatcttgcctacctgcctgtgcgtatggacctgcagtatccagtgggtacgacttcgaaaaagagggatattccttggtgggaatagaccctttcaagctacttcaaaatagccaaatatacagcttaatcagacctaacgagaatccagcacacaagagtcagctggtgtggatggcatgccattctgctgcatttgaagatttaagattgttaagcttcatcagaggaacaaaagtatctcctcgggggaaactgtcaactagaggagtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaagaacttcagacatgagggcagaaatcataagaatgatggaaggtgcaaaaccagaagaagtgtcattccgggggaggggagttttcgagctctcagacgagaaggcaacgaacccgatcgtgccctcttttgacatgagtaatgaaggatcttatttcttcggagacaatgcagaagagtacgacaattaa

SEQ ID NO: 20 - protein, 498 aa, Artificial Sequence
  NP consensus sequence
MASQGTKRSYEQMETDGDRQNATEIRASVGKMIDGIGRFYIQMCTELKLSDHEGRLIQNSLTIEKMVLSAFDERRNKYLEEHPSAGKDPKKTGGPIYRRVDGKWMRELVLYDKEEIRRIWRQANNGEDATAGLTHIMIWHSNLNDATYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACAYGPAVSSGYDFEKEGYSLVGIDPFKLLQNSQIYSLIRPNENPAHKSQLVWMACHSAAFEDLRLLSFIRGTKVSPRGKLSTRGVQIASNENMDNMGSSTLELRSGYWAIRTRSGGNTNQQRASAGQTSVQPTFSVQRNLPFEKSTIMAAFTGNTEGRTSDMRAEIIRMMEGAKPEEVSFRGRGVFELSDEKATNPIVPSFDMSNEGSYFFGDNAEEYDN

SEQ ID NO: 21 - protein, 5 aa, Artificial Sequence
  Linker peptide
GSASG

SEQ ID NO: 22 - protein, 5 aa, Artificial Sequence
  Linker peptide
SGSGA

SEQ ID NO: 23 - protein, 5 aa, Artificial Sequence
  Linker peptide
SAGSG

SEQ ID NO: 24 - protein, 4 aa, Artificial Sequence
  Linker peptide
GGGS

SEQ ID NO: 25 - protein, 8 aa, Artificial Sequence
  Linker peptide
GGSGGGGS

SEQ ID NO: 26 - protein, 14 aa, Artificial Sequence
  T cell epitope
QVHFQPLPPAVVKL

SEQ ID NO: 27 - protein, 14 aa, Artificial Sequence
  T cell epitope
QFIKANSKFIGITE

SEQ ID NO: 28 - protein, 4 aa, Artificial Sequence
  Epitope
AWWP

SEQ ID NO: 29 - DNA, 40 bp, Artificial Sequence
  Promoter
aaaaattgaaattttattttttttttttggaatataaata

SEQ ID NO: 30 - DNA, 70 bp, Artificial Sequence
  Promoter
aaaaaatgaaaataaatacaaaggttcttgagggttgtgttaaattgaaagcgagaaataatcataaatt

SEQ ID NO: 31 - DNA, 7 bp, Artificial Sequence
  Transcription terminal signal
tttttnt

SEQ ID NO: 32 - DNA, 29 bp, Artificial Sequence
  Primer
ggagctccactatttagttggtggtcgcc

SEQ ID NO: 33 - DNA, 27 bp, Artificial Sequence
  Primer
cgggtaccctagtttccggtgaatgtg

SEQ ID NO: 34 - DNA, 23 bp, Artificial Sequence
  Primer
ctagatcatcgtatggagagtcg

SEQ ID NO: 35 - DNA, 17 bp, Artificial Sequence
  Primer
gaaagttttataggtag

SEQ ID NO: 36 - DNA, 34 bp, Artificial Sequence
  Primer
gccaccgcggtggccagccaccgaaagagcaatc

SEQ ID NO: 37 - DNA, 28 bp, Artificial Sequence
  Primer
ggaagatctcaattaacgatgagtgtag

SEQ ID NO: 38 - DNA, 20 bp, Artificial Sequence
  Primer
gatgtaggcgaatttggatc

SEQ ID NO: 39 - DNA, 20 bp, Artificial Sequence
  Primer
tggtaatcgtgtcatattag

SEQ ID NO: 40 - DNA, 21 bp, Artificial Sequence
  Primer
cattattatcggttacacttc

SEQ ID NO: 41 - DNA, 17 bp, Artificial Sequence
  Primer
caagaagtgcttatgag

SEQ ID NO: 42 - DNA, 20 bp, Artificial Sequence
  Primer
ggttccgactttctctcact

SEQ ID NO: 43 - DNA, 24 bp, Artificial Sequence
  Primer
ggcacctctcttaagaagtgtaac

SEQ ID NO: 44 - DNA, 19 bp, Artificial Sequence
  Primer
cttacagaagtggagacac

SEQ ID NO: 45 - DNA, 18 bp, Artificial Sequence
  Primer
gtaaggagactcagcttc

SEQ ID NO: 46 - DNA, 20 bp, Artificial Sequence
  Primer
ggatagagatgtttgtgaac

SEQ ID NO: 47 - DNA, 19 bp, Artificial Sequence
  Primer
cttacagaagtggagacac

SEQ ID NO: 48 - DNA, 18 bp, Artificial Sequence
  Primer
gtaaggagactcagcttc

SEQ ID NO: 49 - DNA, 20 bp, Artificial Sequence
  Primer
gttacactcatgcattgatg

SEQ ID NO: 50 - DNA, 21 bp, Artificial Sequence
  Primer
caaatggaaatctaatagcac

SEQ ID NO: 51 - DNA, 1698 bp, Influenza A virus
atgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctaa

SEQ ID NO: 52 - protein, 565 aa, Influenza A virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 53 - DNA, 183 bp, Artificial Sequence
  Pr8 M2e Transmembrane domain
atgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctctttttcaaatgcatttaccgtcgctttaaatacggactgaaataa

SEQ ID NO: 54 - protein, 60 aa, Artificial Sequence
  Pr8 M2e Transmembrane domain
MSLLTEVETPIRNEWGCRCNGSSDPLTIAANIIGILHLTLWILDRLFFKCIYRRFKYGLK

SEQ ID NO: 55 - protein, 163 aa, Artificial Sequence
  M2e Tandem repeats_C
SLLTEVETPTRNEWECRCSDSSDGSASGSLLTEVETPTRNEWECKCIDSSDSGSGASLLTEVETPIRNEWGCRCNGSSDSAGSGSLLTEVETPIRNEWGCRCNDSSDGSASGSLLTEVETLTRNGWECRCSDSSDSGSGASLLTEVETPTRNGWECKCSDSSD

SEQ ID NO: 56 - protein, 163 aa, Artificial Sequence
  M2e Tandem repeats_S
SLLTEVETPTRNEWESRSSDSSDGSASGSLLTEVETPTRNEWESKSIDSSDSGSGASLLTEVETPIRNEWGSRSNGSSDSAGSGSLLTEVETPIRNEWGSRSNDSSDGSASGSLLTEVETLTRNGWESRSSDSSDSGSGASLLTEVETPTRNGWESKSSDSSD

(19) United States
(12) Patent Application Publication
(10) Pub. No.: US 2013/0115234 A1
(43) Pub. Date: May 9, 2013

(54) Ectodomains of Influenza Matrix 2 Protein, Expression System, and Uses Thereof

(71) Applicants: Tina Guina, Silver Spring, MD (US); Michael Lacy, Damascus, MD (US); Mario Skiadopoulos, Potomac, MD (US); Nutan Mytle, Gaithersburg, MD (US)

(72) Inventors: Tina Guina, Silver Spring, MD (US); Michael Lacy, Damascus, MD (US); Mario Skiadopoulos, Potomac, MD (US); Nutan Mytle, Gaithersburg, MD (US)

(73) Assignee: Emergent Product Development Gaithersburg,Inc., Gaithersburg, MD (US)

(21) Appl. No.: 13/637,305

(22) PCT Filed: Mar. 28, 2011
(86) PCT No.: PCT/US2011/030205

§ 371 Date: Jan. 13, 2013

Related U.S. Application Data
(60) Provisional application No. 61/318,232, filed on Mar. 26, 2010.

(51) Int. Cl. A61K 39/145

(52) U.S. Cl. CPC A61K 39/145
USPC 424/186.1; 435/320.1; 435/349; 435/325; 435/366; 435/69.3; 435/239; 536/23.72; 536/23.2

(57) ABSTRACT

The present invention provides a polynucleotide, polypeptide, recombinant modified vaccinia virus Ankara (rMVA) and related vaccine compositions and methods useful in the prevention and treatment of an influenza viral infection. Provided is an isolated polynucleotide encoding multiple copies of M2 influenza ectodomain peptides or rMVA comprising the polynucleotide. Also provided are methods for inducing an immune response to a subject against an influenza virus or for treating a disease or symptom caused by or resulting from infection with an influenza virus.

### BACKGROUND OF THE INVENTION

Influenza viruses are negative sense RNA members of the Orthomyxoviridae family and cause disease in humans and animals. An influenza infection is common and can be either pandemic or seasonal. Although an influenza infection does not often lead to the death of the infected individual, the morbidity can be severe. As a consequence, influenza epidemics may lead to substantial economic loss. Furthermore, influenza infection can be more dangerous for certain groups of individuals, such as those having suffered from a heart attack, C.A.R.A. patients, or the elderly.

The influenza virus causes disease in a recurring manner due to a complex set of factors including: 1) presence of an established reservoir of influenza A viruses of different subtypes in shorebirds and waterfowl; 2) ability of avian influenza viruses to recombine with influenza viruses of other animals, e.g., swine, a process termed ‘antigenic shift’; 3) accumulation of mutations in viral gene products caused by a lack of proofreading activity of the viral RNA polymerase, a process termed ‘antigenic drift.’ (Tollis, M. and L. Di Trani, Vet. J. 164: 202-215 (2002)). Antigenic shift, antigenic drift and the ability of avian viruses to infect other hosts such as swine and humans results in novel viruses that can cause severe disease in man. These reassortment and mutation events combine to cause the well-characterized antigenic variability in the two surface glycoproteins of the virus, hemagglutinin (HA) and neuraminidase (NA) which provides the virus a mechanism for escaping immune responses, particularly neutralizing antibodies, induced as the result of previous infections or vaccinations. (Palese, P. and J. F. Young, Science 215:1468-1474; Gorman, O. T., et al., Curr. Top. Microbiol. Immunol 176:75-97; and Yewdell, et al., Nature 279: 246-248).

To predict the specific influenza subtypes likely to have global impact on human health, influenza vaccine production must rely on surveillance programs. The time required to produce subtype-matched vaccines, composed of inactivated or ‘split’ virions, typically requires a minimum of 6-8 months. In the face of a serious influenza virus pandemic caused by a viral subtype, this lag time could allow for national or international spread with excessive morbidity and mortality. Therefore, an influenza vaccine that is effective for different subtypes of influenza viruses is highly desirable.

An influenza virus contains eight segments of single-stranded RNA—the genetic instructions for making the virus. Its surface is covered by a layer of two different glycoproteins: one is composed of the molecule hemagglutinin (HA), the other of neuraminidase (NA). The viral capsid is comprised of viral ribonucleic acid and several so called “internal” proteins (polymerases (PB1, PB2, and PA, matrix protein (M1) and nucleoprotein (NP)). Because antibodies against HA and NA have traditionally proved the most effective in fighting infection, much research has focused on the structure, function, and genetic variation of those molecules.

Unlike HA and NA, the external domain of the transmembrane viral M2 protein (M2e) is highly conserved and antibodies directed to this epitope are protective in mice (Treanor, J. J., et al., J. Virol. 64:1375-1377 (1990); Frace, A. M., et al., Vaccine 17:2237-2244 (1999); Wanli, L., et al., Immunol. Lett. 93:131-136 (2004); and Fan, J., et al., Vaccine 22:2293-3003 (2004)). The M2 protein is an integral membrane protein of an influenza A virus that is expressed at the plasma membrane in virus-infected cells. Due to the low abundance of the protein in the virus, the mechanism of protection of the antibody response directed against this epitope is not mediated via viral neutralization but rather by antibody-dependent, cell-mediated cytotoxicity. (Jegerlehner, A., et al., J. Immunol. 172:5598-5605 (2004)).

A major obstacle to the development of vaccines that induce immune responses is the selection of a suitable delivery format. DNA plasmid vaccines and viral vectors, used either alone or together, and recombinant protein or peptides are logical vaccine delivery formats; however, each format has advantages and disadvantages. For example, DNA vaccines are readily produced and safe to administer but potency has been lacking, especially in clinical trials, requiring the administration of large (milligram) doses. Liu, M. A. J. Intern. Med. 253:402-410 (2003). The use of viral vectors to deliver vaccines has raised concerns, usually related to safety and pre-existing immunity to the vector. Therefore, there are ample needs to develop a new influenza vaccine that is safe and effective.

### SUMMARY OF THE INVENTION

The present invention provides an isolated polynucleotide comprising a coding region encoding a polypeptide, wherein said polypeptide comprises at least five influenza virus Matrix 2 protein (M2) ectodomain peptides. In one embodiment, the polypeptide comprises any five or more of the following amino acid sequences arranged in any order relative to each other: (i) SEQ ID NO: 1 (M2e#1_C); (ii). SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S).

Also provided is an isolated polynucleotide comprising a coding region encoding a polypeptide, wherein said polypeptide comprises any three or more of the following M2 ectodomain peptides arranged in any order relative to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S). In one embodiment, the polynucleotide comprises a coding region encoding a polypeptide, which comprises the following six M2 ectodomain peptides arranged in any order relative to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); and (vi) SEQ ID NO: 6 (M2e#6_C) or amino acid sequences of (i) SEQ ID NO: 7 (M2e#1-S); (ii) SEQ ID NO: 8 (M2e#2_S); (iii) SEQ ID NO: 9 (M2e#3_S); (iv) SEQ ID NO: 10 (M2e#4_S); (v) SEQ ID NO: 11 (M2e#5_S); and (vi) SEQ ID NO: 12 (M2e#6_S).

In certain embodiments, the polynucleotide of the invention encodes a polypeptide comprising six M2 ectodomain peptides arranged in any order relative to each other, at least one linker peptide interposed between at least two M2 ectodomain peptides, and optionally an epitope interposed between at least two M2 ectodomain peptides. The epitope can be a T-cell epitope or B-cell epitope.

In some embodiments, the invention is directed to a vector comprising the polynucleotide encoding a polypeptide, which comprises multiple copies of M2 ectodomain peptides. The vector can be a viral vector, e.g., a vaccinia virus vector, e.g., a modified vaccinia virus Ankara (MVA). The vector of the invention can further express an additional polypeptide, e.g., an influenza protein or a fragment thereof. The additional influenza protein can be selected from the group consisting of hemagglutinin (HA), neuraminidase (NA), nucleoprotein (NP), Matrix 1 protein (M1), Matrix 2 protein (M2), non-structural protein (NS), RNA polymerase PA subunit (PA), RNA polymerase PB1 subunit (PB1), RNA polymerase PB2 subunit (PB2), and two or more combinations thereof. In other embodiments, the invention is a host cell comprising the vector and an isolated polypeptide encoded by the polynucleotide.

Also included is a composition comprising the polynucleotide, the vector, e.g., MVA, the host cell, or the polypeptide, and a pharmaceutically acceptable carrier. In one embodiment, a vaccine composition of the present invention further comprises an additional influenza vaccine composition. The additional influenza vaccine can comprise an MVA expressing an influenza protein or a fragment thereof.

In a further embodiment, the present invention includes a method of inducing an immune response against an influenza virus in a subject in need thereof comprising administering to said subject an effective amount of the polynucleotide, the vector, the host cell, the polypeptide, the composition, or any combination thereof either simultaneously or in any order. Also provided is a method for treating, preventing, or reducing the symptoms of an influenza virus infection or a condition associated with an influenza virus infection in a subject in need thereof comprising administering to said subject an effective amount of the polynucleotide, the vector, the host cell, the polypeptide, the composition, or any combination thereof either simultaneously or in any order. The present invention is also directed to a method to attenuate or ameliorate a symptom caused by an influenza virus infection or a condition associated with an influenza virus infection in a subject in need thereof comprising administering to said subject an effective amount of the polynucleotide, the vector, the host cell, the polypeptide, the composition, or any combination thereof either simultaneously or in any order. In other embodiments, the invention includes a method of vaccinating a subject in need thereof against an influenza virus infection comprising administering to said subject an effective amount of the polynucleotide, the vector, the host cell, the polypeptide, the composition, or any combination thereof either simultaneously or in any order.

The sequence identifiers used herein are as follows:

SEQ ID NO: 1: an amino acid sequence of M2 ectodomain #1 having cysteines (M2e#1_C)

SEQ ID NO: 2: an amino acid sequence of M2 ectodomain #2 having cysteines (M2e#2_C)

SEQ ID NO: 3: an amino acid sequence of M2 ectodomain #3 having cysteines (M2e#3_C)

SEQ ID NO: 4: an amino acid sequence of M2 ectodomain #4 having cysteines (M2e#4_C)

SEQ ID NO: 5: an amino acid sequence of M2 ectodomain #5 having cysteines (M2e#5_C)

SEQ ID NO: 6: an amino acid sequence of M2 ectodomain #6 having cysteines (M2e#6_C)

SEQ ID NO: 7: an amino acid sequence of M2 ectodomain #1 having serine substitutions (M2e#1_S)

SEQ ID NO: 8: an amino acid sequence of M2 ectodomain #2 having serine substitutions (M2e#2_S)

SEQ ID NO: 9: an amino acid sequence of M2 ectodomain #3 having serine substitutions (M2e#3_S)

SEQ ID NO: 10: an amino acid sequence of M2 ectodomain #4 having serine substitutions (M2e#4_S)

SEQ ID NO: 11: an amino acid sequence of M2 ectodomain #5 having serine substitutions (M2e#5_S)

SEQ ID NO: 12: an amino acid sequence of M2 ectodomain #6 having serine substitutions (M2e#6_S)

SEQ ID NO: 13: a nucleic acid sequence encoding Matrix 2 (M2) protein of Influenza A/Puerto Rico/8/34 (H1N1)

SEQ ID NO: 14: an amino acid sequence of M2 protein of Influenza A/Puerto Rico/8/34 (H1N1)

SEQ ID NO: 15: a nucleic acid sequence encoding the METR_C polypeptide

SEQ ID NO: 16: an amino acid sequence of the METR_C polyepeptide

SEQ ID NO: 17: a nucleic acid sequence encoding the METR_S polypeptide

SEQ ID NO: 18: an amino acid sequence of the METR_S polyepeptide

SEQ ID NO: 19: a nucleic acid sequence encoding the NP consensus sequence

SEQ ID NO: 20: an amino acid sequence of the NP consensus sequence

SEQ ID NOs: 21-25: linker peptides

SEQ ID NOs: 26-27: T cell epitopes

SEQ ID NO: 28: artificial sequence

SEQ ID NOs: 29-30: promoters

SEQ ID NO: 31: transcription termination signal

SEQ ID NOs: 32-50: primers

SEQ ID NO: 51: a nucleic acid sequence encoding the HA protein of Influenza A/Puerto Rico/8/34 (H1N1)

SEQ ID NO: 52: an amino acid sequence encoding the HA protein of Influenza A/Puerto Rico/8/34 (H1N1)

SEQ ID NO: 53: a nucleic acid sequence encoding the transmembrane domain of M2 protein of Influenza A/Puerto Rico/8/34 (H1N1)

SEQ ID NO: 54: an amino acid sequence of the trasmembrane domain of M2 protein of Influenza A/Puerto Rico/8/34 (H1N1)

SEQ ID NO: 55: an amino acid sequence of the METR_C polypeptide

SEQ ID NO: 56: an amino acid sequence of the METR_S polypeptide

### BRIEF DESCRIPTION OF THE DRAWINGS

FIG. 1 is a schematic vector map of recombination vector vEM011. The abbreviations are as follows: the term “AmpR” represents an Ampicillin resistance gene for selection in bacteria; the term “Flank 1/Flank 2 Del III” represents sequences homologous to the flanking regions of deletion site III of the MVA genome; the term “Ps” means a strong synthetic promoter; BsdR means a gene coding for blasticidine resistance; the term “GFP” means a gene coding for Green Fluorescent Protein; the term “F1 Del3 rpt” represents repeats of the rear part of Flank 1 Del III; and the term “LacZ” means an E. coli Lac Z gene for detection in bacteria.

FIG. 2 shows schematic vector maps of recombination vectors containing the influenza A genes: (A) vEM47 coding for the NP consensus sequence; (B) vEM57 coding for the M2 ectodomain tandem repeat (METR_C) peptide; (C) vEM58 coding for the M2 ectodomain tandem repeat—serine substituted (METR_S) peptide; (D) vEM61 coding for the full-length Matrix 2 domain of influenza virus A/Puerto Rico/8/34 (Pr8M2); (E) vEM62 coding for the transmembrane domain of the Matrix 2 domain derived from influenza virus A/Puerto Rico/8/34 (Pr8M2e-TML); and (F) vEM65 coding for the hemagglutinin protein of influenza virus A/Puerto Rico/8/34 (Pr8HA).

FIG. 3 shows PCR products of various MVAtors: (A) MVAtor-NP consensus (mEM10); (B) MVAtor-METR_C (mEM18); (C) MVAtor-METR_S (mEM19); and (D) MVAtor-Pr8M2 (mEM22), MVAtor-Pr8M2e_TML (mEM23) and MVAtor-Pr8HA (mEM17)

FIG. 4 shows a schematic diagram of exemplary PCR fragment. The abbreviations are as follows: the term “Flank1 Del3” means a flanking sequence 1 of insertion site deletion 3; the term “Ps” means a strong synthetic Vaccinia virus promoter; the term “flu gene” means a gene coding for the gene of interest, i.e., HA, NP, M2e, M2e_TML and METR, respectively; and the term “Flank2 Del3” represents a flanking sequence 2 of deletion 3.

FIG. 5 represents Western blot analysis of influenza proteins expressed by various MVAtors: MVAtor-NP consensus (mEM10), MVAtor-METR_C (mEM18), and MVAtor-METR_S (mEM19).

FIG. 6 represents a haemadsorption assay (HAD) for the CEF cells infected with MVAtor-Pr8HA (MVAtor-Pr8), the mock-infected CEF cells, and the CEF cells infected with MVAtor (MVAtor).

FIG. 7 represents an immunoassay for detection of Pr8M2 in CEF cells: (A) cells infected with MVAtor-Pr8M2; and (B) cells infected with MVAtor-Pr8M2e_TML.

FIG. 8 represents the percent body weight change after immunization with the MVAtors expressing influenza proteins: Pr8M2 (gray, big filled circles), Pr8M2e-TML (black, asterisks), METR-C (gray, small hollow circles), METR-S (gray, filled diamonds), NP consensus (black, filled triangles), MVAtor (black, big hollow circles), and PBS (black, filled small circles).

FIG. 9 represents viral burdens in the immunized mice. Lung weights from each of four mice per group were obtained to express 50% Tissue Culture Infectious Dose (TCID50) per lung weights.

FIG. 10 represents ELISA of serum from the mice immunized with MVA vaccines using mouse IgG anti-M2e antibody (14C2). M2e peptides used in the experiment (M2e#1 (the fourth row), M2e#4 (the third row), M2e#5 (the second row), and M2e#6 (the first row)) are shown in Table 1.

FIG. 11 shows body weight loss of mice immunized with MVA vaccines by intranasal (IN) delivery (FIG. 11A) and intramuscular (IM) delivery (FIG. 11B).

FIG. 12 represents viral burdens in the mice immunized with MVA vaccines by intranasal delivery (left bar) and intramuscular delivery (right bar).

FIG. 13 represents the percent body weight change in mice after immunization with the MVAtors expressing influenza proteins: HA (gray, filled square), NP (gray, filled triangle), M2 (gray, filled diamond), and M2+NP (gray, asterisk) as well as controls: MVAtor (black, filled diamond) and Non-lethal H1N1 PR8 (black, filled circle).

FIG. 14 represents ELISA results showing anti-NP (IgG anti-NP) immune response for mice immunized with 1d21 MVA, 2d21 MVA, 1d21 MVA+NP, 2421 MVA+NP, 1d21 MVA-M2eA+MVA-NP, 2d21 MVA-M2eA+MVA-NP, 1d21 Non-lethal H1N1 PR8, and 2d21 Non-lethal H1N1 PR8MVA using day 42 sera (pre-challenge).

FIG. 15 represents viral burdens in mice immunized with MVA, MVA-HA, MVA-NP, MVA-M2eA, MVA-M2e+NP and A/PR/8/34 at days 2 (left bar) and 4 (right bar) after challenge with H1N1 PR8 virus. Lung weights from each of mice were obtained to express 50% Tissue Culture Infectious Dose (TCID50) per lung weights.

FIG. 16A represents the percent body weight change after immunization with the MVAtors expressing influenza proteins: MVA-PR8HA (black, small filled diamond), MVA-PR8-M2+NP (black, filled square), and MVA-PR8-C+NP (gray, large filled diamond) as well as control: PBS (black, triangle).

FIG. 16B represents the percent body weight change after immunization with the MVAtors expressing all influenza proteins shown in Table 9 as well as negative controls.

FIG. 17 represents ELISA results showing anti-NP (IgG anti-NP) immune response for mice immunized with ConsNP, PR9M2+ConsNP, PR8M2e-TML+ConsNP, METR-C+ConsNP, and METR-S+ConsNP using day 42 sera (pre-challenge).

FIG. 18 represents ELISA results showing anti-M2 (IgG anti-M2e peptide) immune response for mice immunized with M2, M2-TML, METR-C, METR-S, M2+NP, M2-TML-FNP, METR-C+NP, and METR-S+NP using day 42 sera (pre-challenge).

FIG. 19 represents viral burdens in lungs of mice immunized with PBS (IN), MVAtor, NP, M2, M2e-TML, METR-C, METR-S, HA, and sublethal PR8 (IN) intranasally (left bar) or intramuscularly (right bar) at day 3 after challenge with H1N1 PR8 virus. Lung weights from each of mice were obtained to express 50% Tissue Culture Infectious Dose (TCID50) per lung weights.

FIG. 20A represents the percent body weight change after immunization with the MVAtors expressing influenza proteins: M1+NP+METR-C (black, filled square), M1+NP+M2 (gray, open triangle), M2+NP (gray, X), M1 (black, asterisk), and M1+NP (gray, filled circle) as well as controls: Flulaval (black, filled diamond), MVAtor (black, small filled square), and PBS (black, filled triangle).

FIG. 20B represents the percent survival data for mice immunized with MVAtors expressing influenza proteins: M1+NP+METR-C (gray, filled square), M1+NP+M2 (gray, open triangle), M2+NP (gray, X), M1 (black, asterisk), and M1+NP (gray, filled circle) as well as controls: Flulaval (black, open diamond), MVAtor (black, filled square), and PBS (black, filled diamond).

FIG. 21 represents ELISA results showing anti-NP (left half) and anti-M2 (right half) immune response for mice immunized with PBS, MVAtor, M1+NP+METRC, M1+NP+M2, M2+NP, M1, M1+NP, and Flulaval using day 39 sera (pre-challenge). Horizontal lines indicate group means. IgG anti-M2 levels were not assessed for the M1 treatment group.

FIG. 22A represents ELISA results showing anti-M1 immune response for mice immunized with PBS, MVAtor, M1+NP+METRC, M1+M2+NP, M2+NP, M1, M1+NP, and Flulaval using sera from day 39 (pre-challenge). Horizontal lines indicate group means.

FIG. 22B represents ELISA results showing anti-MVA immune response for mice immunized with MVAtor, M1+M2+NP, M2+NP, M1, and M1+NP using sera from day 21 (post-first immunization) (left half) and day 39 (pre-challenge) (right half). Horizontal lines indicate group means.

FIG. 23 represents viral burdens in lungs of mice immunized with PBS, MVAtor, M1+NP+METRC, M1+NP+M2, M2+NP, M1, M1+NP, and Flulaval at day 2 (left bar) or day 4 (right bar) after challenge with sH1N1 A/Mx/4108/09 virus.

FIG. 24 represents viral burdens in nasal turbinates of mice immunized with PBS, MVAtor, M1+NP+METRC, M1+NP+M2, M2+NP, M1, M1+NP, and Flulaval at day 2 (left bar) or day 4 (right bar) after challenge with sH1N1 A/Mx/4108/09 virus.

### DETAILED DESCRIPTION OF THE INVENTION

The present invention provides a polynucleotide encoding multiple copies of Influenza matrix 2 protein ectodomains, a vector (e.g., MVA) containing the polynucleotide, a polypeptide encoded by the polynucleotide, and related compositions as well as methods of administering the polynucleotide, vector (e.g., MVA), polypeptide, or composition to prevent or treat an influenza virus infection.

Methods of making and using the present invention include all conventional techniques of molecular biology, microbiology, immunology, and vaccination. Such techniques are set forth in the literature including but not limited to e.g. Sambrook Molecular Cloning; A Laboratory Manual, Second Edition (1989) and Third Edition (2001); Genetic Engineering: Principles and Methods, Volumes 1-25 (J. K. Setlow ed, 1988); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hanes & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986). (Sambrook et al. (1989) Molecular Cloning: A Laboratory Manual 2nd ed., Cold Spring Harbor Laboratory Press and Ausubel et al. Eds. (1997) Current Protocols in Molecular Biology, John Wiley & Sons, Inc.).

### DEFINITIONS

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

As used herein, the term “isolated” means that the polynucleotide, polypeptide, or fragment, variant, or derivative thereof as well as modified vaccinia Ankara (MVA) has been removed from other biological materials with which it is naturally associated. An example of an isolated polynucleotide is a recombinant polynucleotide contained in a vector. Further examples of an isolated polynucleotide include recombinant polynucleotides maintained in heterologous host cells or purified (partially or substantially) polynucleotides in solution. Isolated RNA molecules include in vivo or in vitro RNA transcripts of the polynucleotides of the present invention. Isolated polynucleotides or nucleic acids according to the present invention further include such molecules produced synthetically.

As used herein, the term “isolated virus” means that the virus, derivative, or variant thereof has been removed from other biological materials with which it is naturally associated or manipulated recombinantly to include a non-naturally occurring substance. An example of an isolated virus is a virus containing a polynucleotide from a different species that was recombinantly inserted in the viral genome. Further examples of an isolated virus include viruses containing a heterologous polynucleotide and maintained in host cells or purified (partially or substantially) virus in solution.

As used herein, the term “purified” means that the polynucleotide, polypeptide, virus or fragment, variant, or derivative thereof is substantially free of other biological material with which it is naturally associated, or free from other biological materials derived, e.g., from a recombinant host cell that has been genetically engineered to replicate viruses of the invention. For example, a purified virus of the present invention includes a virus that is at least 70-100% pure, i.e., a virus which is present in a composition wherein the virus constitutes 70-100% by weight of the total composition. In some embodiments, the purified virus of the present invention is 75%-99% by weight pure, 80%-99% by weight pure, 90-99% by weight pure, or 95% to 99% by weight pure. The relative degree of purity of a virus of the invention is easily determined by well-known methods.

The term “nucleic acid,” “nucleotide,” or “nucleic acid fragment” refers to any one or more nucleic acid segments, e.g., DNA or RNA fragments, present in a polynucleotide or construct. Two or more nucleic acids of the present invention can be present in a single polynucleotide construct, e.g., on a single plasmid, or in separate (non-identical) polynucleotide constructs, e.g., on separate plasmids. Furthermore, any nucleic acid or nucleic acid fragment may encode a single polypeptide, e.g., a single antigen, cytokine, or regulatory polypeptide, or may encode more than one polypeptide, e.g., a nucleic acid may encode two or more polypeptides. In addition, a nucleic acid may encode a regulatory element such as a promoter or a transcription terminator, or may encode a specialized element or motif of a polypeptide or protein, such as a secretory signal peptide or a functional domain. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batter et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid encompasses polynucleotide, gene, cDNA, messenger RNA (mRNA), plasmid DNA (pDNA), or derivatives of pDNA (e.g., minicircles as described in (Darquet, A-M et al., Gene Therapy 4:1341-1349 (1997)). A nucleic acid may be provided in linear (e.g., mRNA), circular (e.g., plasmid), or branched form as well as double-stranded or single-stranded forms. A nucleic acid may comprise a conventional phosphodiester bond or a non-conventional bond (e.g., an amide bond, such as found in peptide nucleic acids (PNA)). The terms nucleic acid, nucleotide, polynucleotide, DNA and gene are used interchangeably herein.

The term “polynucleotide” is intended to encompass a single nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A polynucleotide may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)).

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

“Codon-optimization” is defined herein as modifying a nucleic acid sequence for enhanced expression in a specified host cell by replacing at least one, more than one, or a significant number, of codons of the native sequence with codons that are more frequently or most frequently used in the genes of that host. Various species exhibit particular bias for certain codons of a particular amino acid.

The term “additional” as used herein refers to any biological components that are not identical with the subject biological component. The additional components may be host cells, viruses, polypeptides, polynucleotides, genes, or regulatory regions, such as promoters. It is appreciated that the subject component can derive from an influenza virus, an MVA, an M2 ectodomain peptide, or a polynucleotide encoding the M2 ectodomain as appropriate. For example, an “additional polynucleotide” or an “additional nucleic acid” or an “additional gene” or an “additional sequence” or an “exogenous DNA segment” of an M2 ectodomain gene from an influenza virus can be a promoter from a different virus, e.g., cytomegalovirus, or hemagglutinin from the same influenza virus. The term “additional polypeptide,” “additional amino acid sequence,” “additional antigen,” or “additional protein” of the M2 ectodomain from an influenza virus can be a His tag or any influenza viral polypeptides, fragments, variants, derivatives, or analogues thereof. In certain embodiments, an additional polypeptide may be an influenza polypeptide, fragments, variants, derivatives, or analogues thereof. In other embodiments, an additional polypeptide may be an M2 ectodomain polypeptide, fragment, derivative, variant, or analogue thereof.

The term “influenza polypeptides” or “influenza antigens,” as used herein, encompasses any full-length or mature polypeptides present in an influenza virus, and other variants of the full length or mature polypeptides present in an influenza virus, fragments of the full length or mature polypeptides present in an influenza virus, serotypic, allelic, and other variants of fragments of the full length or mature polypeptides present in an influenza virus, derivatives of the full-length or mature polypeptides present in an influenza virus, derivatives of fragments of the full-length or mature polypeptides present in an influenza virus, analogues of the full-length or mature polypeptides present in an influenza virus, analogues of fragments of the full-length or mature polypeptides present in an influenza virus, and chimeric and fusion polypeptides comprising the full length or mature polypeptides present in an influenza virus or one or more fragments of the full length or mature polypeptides present in an influenza virus. Non-limiting examples of Influenza polypeptides are hemagglutinin (HA), neuraminidase (NA), nucleoprotein (NP), Matrix 1 protein (M1), matrix 2 protein (M2), non-structural protein (NS), or one or more of RNA polymerase subunits, i.e., PA, PB1, and PB2.

In one embodiment in accordance with the present invention, an influenza virus polypeptide is an influenza HA protein, fragment, variant, derivative, or analogue thereof, e.g., a polypeptide comprising an amino acid sequence at least 80%, 85%, 90%, 95%, 96%, 97%, 98%, 99%, or 100% identical to a known HA sequence, e.g., SEQ ID NO: 52, wherein the polypeptide is recognizable by an antibody specifically binds to the HA sequence. The HA sequence may be a full-length HA protein which consists essentially of the HA or extracellular (ECD) domain (HA1 and HA2), the transmembrane (TM) domain, and the cytoplasmic (CYT) domain; or a fragment of the entire HA protein which consists essentially of the HA1 domain and the HA2 domain; or a fragment of the entire HA protein which consists essentially of the HA1, HA2 and the TM domain; or a fragment of the entire HA protein which consists essentially of the CYT domain; or a fragment of the entire HA protein which consists essentially of the TM domain; or a fragment of the entire HA protein which consists essentially of the HA1 domain; or a fragment of the entire HA protein which consists essentially of the HA2 domain. The HA sequence may also include an HA1/HA2 cleavage site. The HA1/HA2 cleavage site is preferably located between the HA1 and HA2 sequences, but also can be arranged in any order relative to the other sequences of the polynucleotide or polypeptide construct. The influenza HA sequence may be from a pathogenic virus strain.

In another embodiment, an influenza polypeptide is an influenza nucleoprotein (NP) sequence, fragment, variant, derivative, or analogue thereof, e.g., a polypeptide comprising, consisting essentially of, or consisting of an amino acid sequence that is at least 80%, 85%, 90%, 95%, 96%, 97%, 98%, 99%, or 100% identical to a known NP polypeptide, wherein said polypeptide is recognizable by an antibody specifically binds to the NP polypeptide. In other embodiments, the influenza NP sequence comprises, consists essentially of, or consists of an NP consensus sequence, e.g., SEQ ID NO: 20.

An influenza polypeptide can be a neuraminidase (NA) protein, fragment, variant, derivative, or analogue thereof. The NA protein, located on the envelope of influenza viruses, is known to catalyze removal of terminal sialic acid residues from viral and cellular glycoconjugates. The NA protein of influenza virus A/Puerto Rico/8/1934 (H1N1) has 454 amino acids and Accession number AAM75160.1 in Genbank, which is incorporated herein by reference in its entirety. The NA protein consists of a cytoplasmic domain (amino acids 1-6), a transmembrane domain (amino acids 7-35), and an extracellular domain (amino acids 36-454). Non-limiting examples of an influenza polypeptide comprises, consists essentially of, or consists of an amino acid sequence at least 80%, 85%, 90%, 95%, 96%, 97%, 98%, 99%, or 100% identical to a known influenza neuraminidase (NA) sequence, wherein the polypeptide is recognizable by an antibody specifically binds to the NA protein. The NA sequence may be a full-length NA protein which consists essentially of the NA. The NA sequence may be a polypeptide comprising, consisting essentially of, or consisting of the extracellular domain, the transmembrane (TM) domain, or the cytoplasmic (CYT) domain of an NA sequence. The influenza NA sequence may be from a pathogenic virus strain.

An influenza polypeptide can be a matrix 1 (M1) protein, fragment, variant, derivative, or analogue thereof. Matrix 1 protein plays critical roles in virus replication. M1 of influenza virus A/Puerto Rico/8/1934 (H1N1) has 252 amino acids and Accession number AAM75161.1 in Genbank, which is incorporated herein by reference in its entirety. Non-limiting examples of an influenza polypeptide comprises, consists essentially of, or consists of an amino acid sequence at least 80%, 85%, 90%, 95%, 96%, 97%, 98%, 99%, or 100% identical to a known influenza M1 sequence, wherein the polypeptide is recognizable by an antibody specifically binds to the M1 protein. The M1 sequence may be a polypeptide comprising, consisting essentially of, or consisting of a fragment of an NA sequence.

In other embodiments, an influenza polypeptide can be a non-structural (NS) protein, fragment, variant, derivative, or analogue thereof. Non-structural protein (NS) inhibits post-transcriptional processing of cellular pre-mRNA, by binding and inhibiting two cellular proteins that are required for the 3′-end processing of cellular pre-mRNAs: the 30 kDa cleavage and polyadenylation specificity factor (CPSF4) and the poly(A)-binding protein 2 (PABPN1). The NS protein of influenza virus A/Puerto Rico/8/1934 (H1N1) has 230 amino acids and Accession number AAM75163.1 in Genbank, which is incorporated herein by reference in its entirety. Non-limiting examples of an influenza polypeptide comprises, consists essentially of, or consists of an amino acid sequence at least 80%, 85%, 90%, 95%, 96%, 97%, 98%, 99%, or 100% identical to a known influenza NS sequence, wherein the polypeptide is recognizable by an antibody specifically binds to the NS protein. The NS sequence may be a polypeptide comprising, consisting essentially of, or consisting of a fragment of an NS sequence.

In some embodiments, an influenza polypeptide is an RNA polymerase PA (polymerase acidic protein) subunit, fragment, variant, derivative, or analogue thereof. The PA polypeptide displays an elongation factor activity in viral RNA synthesis. The PA protein of influenza virus A/Puerto Rico/811934 (H1N1) has 716 amino acids and Accession number AAM75157.1 in Genbank, which is incorporated herein by reference in its entirety. Non-limiting examples of an influenza polypeptide comprises, consists essentially of or consists of an amino acid sequence at least 80%, 85%, 90%, 95%, 96%, 97%, 98%, 99%, or 100% identical to a known influenza PA sequence, wherein the polypeptide is recognizable by an antibody specifically binds to the PA protein. The PA sequence may be a polypeptide comprising, consisting essentially of, or consisting of a fragment of a PA sequence.

In certain embodiments, an influenza polypeptide used herein is an RNA polymerase PB1 (polymerase basic protein 1) subunit, fragment, variant, derivative, or analogue thereof. PB1 proteins are responsible for replication and transcription of virus segments. The PB1 protein of influenza virus A/Puerto Rico/811934 (H1N1) has 757 amino acids and Accession number AAM75156.1 in Genbank, which is incorporated herein by reference in its entirety. Non-limiting examples of an influenza polypeptide comprises, consists essentially of, or consists of an amino acid sequence at least 80%, 85%, 90%, 95%, 96%, 97%, 98%, 99%, or 100% identical to a known influenza PB1 sequence, wherein the polypeptide is recognizable by an antibody specifically binds to the PB1 protein. The PB1 sequence may be a polypeptide comprising, consisting essentially of, or consisting of a fragment of a PB1 sequence.

In still other embodiments, an influenza polypeptide can be an RNA polymerase PB2 (polymerase basic protein 2) subunit, fragment, variant, derivative, or analogue thereof. PB2 proteins are involved in transcription initiation and cap-stealing mechanism, in which cellular capped pre-mRNA are used to generate primers for viral transcription. PB2 of influenza virus A/Puerto Rico/8/1934 (H1N1) has 759 amino acids and Accession number AAM75155.1 in Genbank, which is incorporated herein by reference in its entirety. Non-limiting examples of an influenza polypeptide comprises, consists essentially of, or consists of an amino acid sequence at least 80%, 85%, 90%, 95%, 96%, 97%, 98%, 99%, or 100% identical to a known influenza PB2 sequence, wherein the polypeptide is recognizable by an antibody specifically binds to the PB2 protein. The PB2 sequence may be a polypeptide comprising, consisting essentially of, or consisting of a fragment of a PB2 sequence.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example, promoters, ribosome binding sites, transcriptional terminators, and the like, are outside the coding region.

The term “fragment,” “analog,” “derivative,” or “variant” when referring to an influenza polypeptide includes any polypeptides which retain at least some of the immunogenicity or antigenicity of the naturally-occurring influenza proteins. Fragments of influenza polypeptides of the present invention include proteolytic fragments, deletion fragments and in particular, fragments of influenza polypeptides which exhibit increased solubility during expression, purification, and or administration to an animal. Fragments of influenza polypeptides further include proteolytic fragments or deletion fragments which exhibit reduced pathogenicity when delivered to a subject. Polypeptide fragments further include any portion of the polypeptide which comprises an antigenic or immunogenic epitope of the native polypeptide, including linear as well as three-dimensional epitopes.

An “epitopic fragment” of a polypeptide antigen is a portion of the antigen that contains an epitope. An “epitopic fragment” may, but need not, contain amino acid sequence in addition to one or more epitopes.

The term “variant,” as used herein, refers to a polypeptide that differs from the recited polypeptide due to amino acid substitutions, deletions, insertions, and/or modifications. Variants may occur naturally, such as a subtypic variant. The term “subtypic variant” is intended polypeptides or polynucleotides that are present in a different influenza virus subtypes including, but not limited to, Human A/Puerto Rico/8/34 (H1N1), Human ANiet Nam/1203/2004 (H5N1), Human A/Hong Kong/156/97 (H5N1), Human A/Hong Kong/483/97 (H5N1), Human A/Hong Kong/1073/99 (H9N2), Avian A/Chicken/HK/G9/97 (H9N2), Swine A/Swine/Hong Kong/10/98 (H9N2), Avian A/FPV/Rostock/34 (H7N1), Avian A/Turkey/Italy/4620/99 (H7N1), Avian A/FPV/Weybridge/34 (H7N7), Human A/New Calcdonia/20/99 (H1N1), Human A/Hong Kong/1/68 (H3N2), Human A/Shiga/25197 (H3N2), Human A/Singapore/1/57 (H2N2), Human A/Leningrad/134/57 (H2N2), Human A/Ann Arbor/6/60 (H2N2), Human A/Brevig Mission/1/18 (H1N1), Swine A/Swine/Wisconsin/464/98 (H1N1), Human A/Netherlands/219/03 (H7N7) and Human A/Wyoming/3/2003 (H3N2). The subtypic variants are naturally occurring variants, but it can also be produced using art-known mutagenesis techniques.

Non-naturally occurring variants may be produced using art-known mutagenesis techniques. In one embodiment, variant polypeptides differ from an identified sequence by substitution, deletion, or addition of five amino acids or fewer. Such variants may generally be identified by modifying a polypeptide sequence, and evaluating the antigenic properties of the modified polypeptide using, for example, the representative procedures described herein.

Polypeptide variants exhibit at least about 60-70%, for example, 75%, 80%, 85%, 90%, 94%, 95%, 96%, 97%, 98%, 99%, or 99.9% sequence identity with identified polypeptides. Variant polypeptides may comprise conservative or non-conservative amino acid substitutions, deletions or additions. Derivatives of polypeptides are polypeptides which have been altered so as to exhibit additional features not found on the native polypeptide. Examples include fusion proteins. An analog is another form of a polypeptide of the present invention. An example is a proprotein which can be activated by cleavage of the proprotein to produce an active mature polypeptide.

Variants may also, or alternatively, contain other modifications, whereby, for example, a polypeptide may be conjugated or coupled, e.g., fused to an additional polypeptide, e.g., a signal (or leader) sequence at the N-terminal end of the protein which co-translationally or post-translationally directs transfer of the protein. The polypeptide may also be conjugated or produced coupled to a linker or other sequence for ease of synthesis, purification or identification of the polypeptide (e.g., 6-His), or to enhance binding of the polypeptide to a solid support. For example, a polypeptide may be conjugated or coupled to an immunoglobulin Fc region. The polypeptide may also be conjugated or coupled to a sequence that imparts or modulates the immune response to the polypeptide (e.g., a T-cell epitope, B-cell epitope, cytokine, chemokine, etc.) and/or enhances uptake and/or processing of the polypeptide by antigen presenting cells or other immune system cells. The polypeptide may also be conjugated or coupled to other polypeptides/epitopes from influenza virus and/or from other bacteria and/or other viruses to generate a hybrid immunogenic protein that alone or in combination with various adjuvants can elicit protective immunity to other pathogenic organisms.

The term “sequence identity” as used herein refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window may comprise additions or deletions termed gaps while the reference sequence is kept constant. An optimal alignment is that alignment which, even with gaps, produces the greatest possible number of “identical” positions between the reference and comparator sequences. The terms “sequence identity” and “identical” are used interchangeably herein. Accordingly, sequences sharing a percentage of “sequence identity” are understood to be that same percentage “identical.” Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10), and any other required parameter including but not limited to matrix option.

The term “epitopes,” as used herein, refers to portions of a polypeptide having antigenic or immunogenic activity in an animal, for example a mammal, for example, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind its antigen as determined by any method well known in the art. Immunospecific binding excludes non-specific binding but does not necessarily exclude cross-reactivity with other antigens. Whereas all immunogenic epitopes are antigenic, antigenic epitopes need not be immunogenic.

An “effective amount” is that amount the administration of which to an individual, either in a single dose or as part of a series, is effective for treatment or prevention. An amount is effective, for example, when its administration results in a reduced incidence of influenza infections relative to an untreated individual, as determined two weeks after challenge with an infectious influenza virus. This amount varies depending upon the health and physical condition of the individual to be treated, the taxonomic group of individual to be treated (e.g. human, nonhuman primate, primate, etc.), the responsive capacity of the individual's immune system, the degree of protection desired, the formulation of the vaccine, a professional assessment of the medical situation, and other relevant factors. It is expected that the effective amount will fall in a relatively broad range that can be determined through routine trials. Typically a single dose is from about 10 μg to 10 mg of MVA/kg body weight or an amount of a modified carrier organism or host cell, sufficient to provide a comparable quantity of recombinantly expressed influenza polypeptide.

The term “subject” is meant any subject, particularly a mammalian subject, for whom diagnosis, prognosis, immunization, or therapy is desired. Mammalian subjects include, but are not limited to, humans, domestic animals, farm animals, zoo animals such as bears, sport animals, pet animals such as dogs, cats, guinea pigs, rabbits, rats, mice, horses, cattle, bears, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the animal is a human subject.

The term “animal” is intended to encompass a singular “animal” as well as plural “animals” and comprises mammals and birds, as well as fish, reptiles, and amphibians. The term animal also encompasses model animals, e.g., disease model animals. In some embodiments, the term animal includes valuable animals, either economically or otherwise, e.g., economically important breeding stock, racing animals, show animals, heirloom animals, rare or endangered animals, or companion animals. In particular, the mammal can be a human subject, a food animal or a companion animal.

As used herein, a “subject in need thereof” refers to an individual for whom it is desirable to treat, i.e., to prevent, cure, retard, or reduce the severity of influenza infections, and/or result in no worsening of symptoms of influenza infections over a specified period of time.

The terms “prime” or “priming” or “primary” and “boost” or “boosting” as used herein to refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously.

The term “passive immunity” refers to the immunity to an antigen developed by a host animal, the host animal being given antibodies produced by another animal, rather than producing its own antibodies to the antigen. The term “active immunity” refers to the production of an antibody by a host animal as a result of the presence of the target antigen.

As used herein, an “immune response” refers to a response in the recipient to the introduction of the polynucleotide, polypeptide, attenuated poxviruses, e.g., MVA, or composition of the present invention, generally characterized by, but not limited to, production of antibodies and/or T cells. Generally, an immune response may be a cellular response such as induction or activation of CD4+T cells or CD8+T cells or both, specific for influenza M2 ectodomain or M2 ectodomain tandem repeat (METR), a humoral response of increased production of influenza M2 ectodomain-specific or METR-specific antibodies, or both cellular and humoral responses. The immune response established by the vaccine comprising an MVA of the invention includes but is not limited to responses to proteins expressed by host cells after the MVA has entered host cells. In the instant invention, upon subsequent challenge by infectious organisms (e.g., influenza A virus), the immune response prevents formation or development of influenza viral particles. Immune responses can also include a mucosal response, e.g., a mucosal antibody response, e.g., S-IgA production or a mucosal cell-mediated response, e.g., T-cell response.

“Vaccine” as used herein is a composition comprising an immunogenic agent and a pharmaceutically acceptable diluent in combination with excipient, adjuvant, additive and/or protectant. The immunogen may be comprised of a whole infectious agent or a molecular subset of the infectious agent (produced by the infectious agent, synthetically or recombinantly including without limitation, polypeptides or polynucleotides). For example, an “MVA vaccine” as used herein means a composition comprising an isolated MVA comprising at least one polynucleotide encoding multiple copies of M2 ectodomain peptides (e.g., M2e Tandem Repeat) and a pharmaceutically acceptable diluent in combination with excipient, adjuvant, additive and/or protectant. When the vaccine is administered to a subject, the immunogen stimulates an immune response that will, upon subsequent challenge with infectious agent, protect the subject from illness or mitigate the pathology, symptoms or clinical manifestations caused by that agent. The vaccine, according to the invention, can be either therapeutic or prophylactic. A therapeutic (treatment) vaccine is given after infection and is intended to reduce or arrest disease progression. A preventive (prophylactic) vaccine is intended to prevent initial infection or reduce the burden of the infection. Agents used in vaccines against an influenza related disease may be an attenuated influenza virus, or purified or artificially manufactured molecules associated with the influenza virus, e.g., recombinant proteins, synthetic peptides, DNA plasmids, and recombinant viruses or bacteria expressing influenza proteins. A vaccine may further comprise other components such as excipient, diluent, carrier, preservative, adjuvant or other immune enhancer, or combinations thereof, as would be readily understood by those in the art.

A multivalent vaccine refers to any vaccine prepared from two or more poxviruses, e.g., MVAs, each of them expressing different antigens, e.g., different influenza antigens. Alternatively, a multivalent vaccine comprises a single isolated poxvirus, e.g., MVA comprising polynucleotides encoding two or more antigens, e.g., influenza antigens that are not identical. The two or more antigens, e.g., influenza antigens, may be derived from the same polypeptide but contain different epitopes that may induce an immune response that is not cross reactive.

The term “immunogenic carrier” as used herein refers to a first polypeptide or fragment, variant, or derivative thereof which enhances the immunogenicity of a second polypeptide, e.g., an antigenic epitope, or fragment, variant, or derivative thereof.

The term “adjuvant” refers to any material having the ability to (1) alter or increase the immune response to a particular antigen or (2) increase or aid an effect of a pharmacological agent. As used herein, any compound which may increase the expression, antigenicity or immunogenicity of an MVA of the invention is a potential adjuvant. In some embodiments, the term adjuvant refers to a TLR stimulating adjuvant, wherein the TLR adjuvant includes compounds that stimulate the TLR receptors (e.g., TLR1-TLR13), resulting in an increased immune system response to the vaccine composition of the present invention. TLR adjuvants include, but are not limited to, CpG and MPL.

The term “attenuate” as used herein includes rendering an infectious agent, e.g., a poxvirus, e.g., MVA, unable to replicate in at least one host cells, e.g., any mammalian cells, e.g., any human cells. An attenuated MVA may still have a limited capacity to replicate in certain mammalian cells, e.g., BS-C-1 and CV-1 cells. While being replication incompetent in a certain mammalian cell, the MVA may still retain a full capacity to replicate in other mammalian cells (e.g., BHK-21 cells) as well as avian cells, for example, primary or immortalized chick or duck cells, e.g., AGE1cr, AGE1cr.pIX, or EB66®. Cell replication cycles of attenuated MVA may be blocked in any stages of its life cycle. For example, an attenuated MVA have a cell replication cycle blocked in a later stage which prevents new viruses from being generated and released. The term “replicate” or “replicating” as used herein refers to an ability to progress through some portion of a viral life cycle, e.g., transcription and translation of viral gene products and nucleic acid replication and also in some instances, an ability to produce or develop mature infectious virions.

### Polynucleotides

The present invention provides an isolated polynucleotide comprising a coding region, which encodes a polypeptide comprising multiple copies of the ectodomains of Influenza virus Matrix 2 protein to induce an immune response against an influenza virus.

Influenza virus Matrix 2 protein (“M2”) is a proton-selective ion channel protein, which is integral in the viral envelope of the influenza A virus. The channel itself is a homotetramer, in which the units form helices stabilized by two disulfide bonds. The M2 protein unit consists of three protein domains: the ectodomain having the 24 amino acids on the N-terminal end, which is exposed to the outer environment, the transmembrane region having the 19 hydrophobic amino acids, and the cytoplasmic domain having the 54 amino acids on the C-terminal end. The M2 protein has an important role in the life cycle of the influenza A virus. Located in the viral envelope, the protein enables hydrogen ions to enter the viral particle (virion) from the endosome, thus lowering the pH inside of the virus, which causes dissociation of the viral matrix protein M1 from the ribonucleoprotein RNP. This is a crucial step in uncoating of the virus and exposing its content to the cytoplasm of the host cell.

The present invention provides an isolated polynucleotide, which comprises a coding region encoding a polypeptide, wherein the polypeptide comprises at least five, at least six, at least seven, at least eight, at least nine, at least ten, at least eleven, or at least twelve Influenza virus Matrix 2 protein (M2) ectodomain peptides, variants, fragments, derivatives, or analogues thereof. The M2 ectodomain peptides therein can be arranged or combined in any order relative to each other. In one embodiment, a polynucleotide of the present invention comprises a coding region encoding a polypeptide, which comprises at least six M2 ectodomain peptides, fragments, variants, fragments, derivatives, or analogues thereof, wherein the at least six M2 ecdotomain peptides are arranged or combined in any order relative to each other. The term “multiple copies of the M2 ectodomain peptides” is also used interchangeably herein as “M2e tandem repeat,” “M2 ectodomain tandem repeat,” or “METR.”

A Matrix 2 (M2) protein used for the instant invention can be obtained from any influenza A virus including, but not limited to, Human A/Puerto Rico/8/34 (H1N1), Human A/Viet Nam/1203/2004 (H5N1), Human A/Viet Nam/DT-036/2005 (H5N1), Human A/Grebe/Novoslbirsk/29/2005 (H5N1), Avian A/Bar-headed Goose/Mongolia/1/05 (H5N1), A/cat/Thailand/KU-02/04 (H5N1), Human A/Hong Kong/213/03 (H5N1), Avian A/chicken/Guandong/174/04 (H5N1), Human A/Hong Kong/156/97 (H5N1), Human A/Hong Kong/483/97 (H5N1), Avian A/Quail/Hong Kong/G1/97 (H9N2), Avian A/Duck/Hong Kong/Y260/97 (H9N2), Avian A/chicken HK/FY23/03 (H9N2), Avian A/turkey/Germany/3/91 (H1N1), Human A/Hong Kong/1073/99 (H9N2), Avian A/Chicken/HK/G9/97 (H9N2), Swine A/Swine/Hong Kong/10/98 (H9N2), Swine A/Swine/Saskatchewan/18789/02 (H1N1), Avian A/mallard/Alberta/1302003 (H1N1), Avian A/mallard/NY/6750/78 (H2N2), Avian A/mallard/Potsdam/177-4/83 (H2N2), Avian/A/duck/Hokkaldo/95/2001 (H2N2), Avian A/Duck/Korea/S9/2003 (H3N2), Swine A/swine/Shandong/2/03 (H5N1), Avian A/Chicken/California/0139/2001 (H6N2), Avian. A/Guillemot/Sweden/3/2000 (H6N2), Avian A/Goose/Hong Kong/W 217/97 (H6N9), Avian A/chicken/British Columbia/04 (H7N3), Avian A/Shorebird/Delaware/9/96 (H9N2), Avian A/Duck/Hong Kong/Y439/97 (H9N2), Avian A/Teal/Hong Kong/W312/97 (H6N1), Swine A/swine/Korea/S452/2004 (H9N2), Avian A/chicken/Netherlands/1/2003 (H7N7), Avian A/mallard/Alberta/2001 (H1N1), Avian A/Duck/Hunan/114/05 (H5N1), Swine A/Swine/Cotes d'Armor/1482/99 (H1N1), Swine A/Swine Belzig/2/2001 (H1N1), Avian A/Turkey/Italy/220158/2002 (H7N3), Avian A/HK12108/2003 (H9N2), Avian AJFFV/Rostock/34 (H7N1), Avian A/Turkey/Italy/4620/99 (H7N1), Avian AJFPV/Weybridge/34 (H7N7), Avian A/FPV/Dobson/27 (H7N7), Human A/New Calcdonia/20/99 (H1N1), Human A/Hong Kong/1/68 (H3N2), Human A/Charlottesville/03/2004 (H3N2), Human A/Canterbury/129/2005 (H3N2), Human A/Shiga/25/97 (H3N2), Human A/Singapore/1/57 (H2N2), Human A/Leningrad/134/57 (H2N2), Human A/Ann Arbor/6/60 (H2N2), Human A/Brevig Mission/1/18 (H1N1), Human A/Canada/720/05 (H2N2), Swine A/Swine/Wisconsin/464/98 (H1N1), Swine A/Swine/Texas/4199-2/98 (H3N2), Avian A/turkey/Ohio/313053/2004 (H3N2), Avian A/Turkey/North Carolina/12344/03 (H3N2), Avian A/Goose/Guangdong/1/98 (H5N1), Human A/Netherlands/219/03 (H7N7), Human A/Willson-Smith/33 (H1N1), Human A/New Calcdonia/20/99 (H1N1), Human A/Japan/305/57 (H2N2), Avian A/chicken/Iran/16/2000 (H9N2), Avian A/mallard/MN/1/2000 (H5N2), A/Leiden/01272/2006 (H3N2), A/Tilburg/45223/2005 (H3N2), A/Pennsylvania/PIT25/2008 (H3N2), A/NYMC X-171A(Puerto Rico/8/1934-Brisbane/10/2007)(H3N2), A/Managua/26/2007 (H3N2), A/Hong Kong/1-1-MA-20D/1968 (H3N2), A/Czech Republic/1/1966 (H2N2), A/chicken/Shanghai/2/1999 (H9N2))], A/Myanmar/M187/2007 (H3N2), A/Guinea fowl/New York/101276-1/2005 (H7N2), Avian A/Muscovy duck/New York/87493-3/2005 (H7N2), Avian A/turkey/New York/122501-2/2005 (H7N2), Avian A/mallard/Italy/4223-2/2006 (H5N2) and Human A/Wyoming/3/2003 (H3N2).

In one embodiment, the ectodomain of the Matrix 2 protein for the present invention is derived from influenza A/Puerto Rico/8/34 (H1N1). The full-length M2 protein of influenza human A/Puerto Rico/8/34 (H1N1) possesses 97 amino acids and is represented as SEQ ID NO: 14. A nucleic acid encoding the M2 protein (SEQ ID NO: 14) is represented herein as SEQ ID NO: 13. The N-terminal sequence exposed on the surface of an influenza virus is 23 amino acids without the N-terminal

Methionine or 24 amino acids with the N-terminal Methionine (the underlined sequence of SEQ ID NO: 14 shown below) and is identified as the ectodomain (“M2e”).

Matrix 2 protein sequence from influenza
A/Puerto Rico/8/34 (H1N1)
(SEQ ID NO: 14)

| | |
|---|---|
| 0 | MSLLTEVETP IRNEWGCRCN GSSDPLTIAA NIIGILHLTL |
| | WILDRLFFKC |
| 50 | IYRRFKYGLK GGPSTEGVPK SMREEYRKEQ QSAVDADDGH |
| | FVSIELE |

M2 ectodomain peptides, variants, derivatives, fragments, or analogues thereof as used herein can comprise an antibody epitope located in the M2 ectodomain peptide, wherein the M2 ectodomain peptide comprises, consists essentially of, or consists of about 8-39 amino acids, about 9-38 amino acids, about 10-37 amino acids, about 11-36 amino acids, about 12-35 amino acids, about 13-34 amino acids, about 14-33 amino acids, about 15-32 amino acids, about 16-31 amino acids, about 17-30 amino acids, about 18-29 amino acids, about 19-28 amino acids, about 20-27 amino acids, about 21-26 amino acids, about 22-25 amino acids, or about 23-24 amino acids. In a specific embodiment, an M2 ectodomain peptide consists essentially of or consists of 23 amino acids. Non-limiting examples of antibody epitopes comprise, consists essentially of, or consists of an amino acid sequence selected from the group consisting of EVETPTRN (amino acids 5-12 of SEQ ID NO: 1), SLLTEVETPT (amino acids 1-10 of SEQ ID NO: 1), ETPTRNEWECK (amino acids 7-17 of SEQ ID NO: 2), EVETPIRNEW (amino acids 5-14 of SEQ ID NO: 3), and LTEVETPIRNEWGCRCN (amino acids 3-19 of SEQ ID NO: 3).

Alternatively, M2 ectodomain peptides can be variants, derivatives, or analogues thereof, which are recognizable by an antibody that specifically binds to a peptide consisting of SEQ ID NOs: 1, 2, 3, 4, 5, or 6. For example, the variants, derivatives, or analogues of the M2 ectodomain peptide is at least 80%, 85%, 90%, 95%, 96%, 97%, 98%, 99%, or 100% identical to SEQ ID NOs: 1, 2, 3, 4, 5, or 6 (M2e#3_C), wherein the variants, derivatives, or analogues are recognizable by an antibody specifically binds to a peptide consisting of SEQ ID NO: 1, 2, 3, 4, 5, or 6. An example of an antibody specifically binds to a peptide consisting of SEQ ID NO: 3 is monoclonal antibody 14C2, which is described in U.S. Pat. No. 5,290,686, incorporated herein by reference in its entirety. Non-limiting examples of the M2 ectodomain peptides are listed in Table 1.

TABLE 1
List of Six Versions of M2e Peptides (C) Utilized in
METR Vaccine

| | | | Prevalence in |
|---|---|---|---|
| Amino Acid Sequence | Peptide | SEQ ID | Naturally Occurring |
| of M2e peptides | designation | NO: | Influenza strains |
| SLLTEVETPTRNEWECRCSDSSD | M2e#1_C | SEQ ID | H5 human 1999 to 2008 |
| | | NO: 1 | (70%) |
| SLLTEVETPTRNEWECKCIDSSD | M2e#2_C | SEQ ID | H5 human 1999 to |
| | | NO: 2 | 2008 (30%) |
| SLLTEVETPIRNEWGCRCNGSSD | M2e#3_C | SEQ ID | H3 human (some H1) |
| | | NO: 3 | |
| SLLTEVETPIRNEWGCRCNDSSD | M2e#4_C | SEQ ID | H1 and H3 human |
| | | NO: 4 | |
| SLLTEVETLTRNGWECRCSDSSD | M2e#5_C | SEQ ID | H9 and H6 human, also |
| | | NO: 5 | avian |
| SLLTEVETPTRNGWECKCSDSSD | M2e#6_C | SEQ ID | Avian H7, also in H3, |
| | | NO: 6 | H8, H10, H2, H6, H9 |

In addition, M2 ectodomain peptides, variants, fragments, derivatives, or analogues as used herein may include, but not limited to, M2 ectodomain peptide variants, in which cysteines (C) in the M2 ectodomain peptides are substituted by serines (S). The substitution prevents disulfide bond formation between the two cysteines but does not affect immunogenicity of the M2 ectodomain peptides. Non-limiting examples of the serine-substituted M2 ectodomain peptides are shown in Table 2:

TABLE 2
List of Six Versions of M2e Peptides (S)
Utilized in METR Vaccine

| Amino Acid Sequence | Peptide | SEQ ID |
|---|---|---|
| of M2e peptides | designation | NO: |
| SLLTEVETPTPNEWESRSSDSSD | M2e#1_S | SEQ ID |
| | | NO: 7 |
| SLLTEVETPTRNEWESKSIDSSD | M2e#2_S | SEQ ID |
| | | NO: 8 |
| SLLTEVETPIRNEWGSRSNGSSD | M2e#3_S | SEQ ID |
| | | NO: 9 |
| SLLTEVETPIRNEWGSRSNDSSD | M2e#4_S | SEQ ID |
| | | NO: 10 |
| SLLTEVETLTRNGWESRSSDSSD | M2e#5_S | SEQ ID |
| | | NO: 11 |
| SLLTEVETPTRNGWESKSSDSSD | M2e#6_S | SEQ ID |
| | | NO: 12 |

In one embodiment, polynucleotides of the invention encode a protein, which comprises an amino acid sequence selected from the group consisting of SEQ ID NO: 7, SEQ ID NO: 8, SEQ ID NO: 11, SEQ ID NO: 12, and a combination of SEQ ID NOs: 7, 8, 11, and 12.

The present invention is directed to an isolated polynucleotide comprising a coding region encoding a polypeptide, wherein the polypeptide comprises at least three of the following M2 ectodomain peptides arranged in any order relative to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S). In one embodiment, the polynucleotide comprises at least four, at least five, at least six, at least seven, at least eight, at least nine, at least ten, at least eleven, or at least twelve of the M2 ectodomain peptides arranged or combined in any order relative to each other:

In another embodiment, a polynucleotide of the invention comprises a nucleic acid sequence encoding a polypeptide, wherein the polypeptide comprises at least six of the following M2 ectodomain peptides arranged in any order relative to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S). In a particular embodiment, the M2 ectodomain peptides in the invention comprises the following amino acid sequences combined or arranged in any order relative to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); and (vi) SEQ ID NO: 6 (M2e#6_C) or an amino acid sequence selected from the group consisting of: (i) SEQ ID NO: 7 (M2e#1_S); (ii) SEQ ID NO: 8 (M2e#2_S); (iii) SEQ ID NO: 9 (M2e#3_S); (iv) SEQ ID NO: 10 (M2e#45); (v) SEQ ID NO: 11 (M2e#5_S); and (vi) SEQ ID NO: 12 (M2e#6_S). In one specific embodiment, an isolated polynucleotide of the instant invention comprises a coding region encoding a polypeptide, which comprises the following amino acid sequences arranged in any order relative to each other: (a) SEQ ID NO: 1, SEQ ID NO: 2, SEQ ID NO: 3, SEQ ID NO: 4, SEQ ID NO: 5, and SEQ ID NO: 6 or (b) SEQ ID NO: 7, SEQ ID NO: 8, SEQ ID NO: 9, SEQ ID NO: 10, SEQ ID NO: 11, and SEQ ID NO: 12. In another specific embodiment, the polypeptide of the invention comprising the multiple copies of the M2 ectodomain peptides (METR) sequence comprises, consists essentially of, or consists of SEQ ID NOs: 1-6. In a still specific embodiment, the METR sequence comprises, consists essentially of, or consists of SEQ ID NO: 16 or SEQ ID NO: 55.

METR_C Sequence
(SEQ ID NO: 16)
SLLTEVETPTRNEWECRCSDSSD GSASG
SLLTEVETPTRNEWECKCIDSSD SGSGA
SLLTEVETPIRNEWGCRCNGSSD SAGSG
SLLTEVETPIRNEWGCRCNDSSD QVHFQPLPPAVVKL
SLLTEVETLTRNGWECRCSDSSD QFIKANSKFIGITE
SLLTEVETPTRNGWECKCSDSSD
METRC Sequence
(SEQ ID NO: 55)
SLLTEVETPTRNEWECRCSDSSD GSASG
SLLTEVETPTRNEWECKCIDSSD SGSGA
SLLTEVETPIRNEWGCRCNGSSD SAGSG
SLLTEVETPIRNEWGCRCNDSSD GSASG
SLLTEVETLTRNGWECRCSDSSD SGSGA
SLLTEVETPTRNGWECKCSDSSD

In a further embodiment, the polypeptide comprising the METR sequence comprises, consists essentially of, or consists of SEQ ID NOs: 7-12. In a particular embodiment, the METR sequence comprises, consists essentially of, or consists of SEQ ID NO: 18 or SEQ ID NO: 56.

METR_S Sequence
(SEQ ID NO: 18)
SLLTEVETPTRNEWESRSSDSSD GSASG
SLLTEVETPTRNEWESKSIDSSD SGSGA
SLLTEVETPIRNEWGSRSNGSSD SAGSG
SLLTEVETPIRNEWGSRSNDSSD QVHFQPLPPAVVKL
SLLTEVETLTRNGWESRSSDSSD QFIKANSKFIGITE
SLLTEVETPTRNGWESKSSDSSD
METR_S Sequence
(SEQ ID NO: 56)
SLLTEVETPTRNEWESRSSDSSD GSASG
SLLTEVETPTRNEWESKSIDSSD SGSGA
SLLTEVETPIRNEWGSRSNGSSD SAGSG
SLLTEVETPIRNEWGSRSNDSSD GSASG
SLLTEVETLTRNGWESRSSDSSD SGSGA
SLLTEVETPTRNEWESKSIDSSD

Also provided is an isolated polynucleotide comprising a coding region, which encodes an influenza nucleoprotein (NP) consensus sequence. Influenza NP proteins are structurally associated with influenza gene (RNA) segments and possess 498 amino acids in length. The primary function of NP is to encapsidate the virus genome for the purpose of RNA transcription, replication, and packaging. The NP gene is relatively well conserved, with a maximum amino acid difference of less than 11% (Shu, L. L., et al., Nucleic Acids Res. 22: 5047-5053 (1993)). The influenza NP consensus sequence for the invention can be obtained by comparing an alignment of 700 of the most frequent NP Influenza A sequences from viruses that emerged recently (2004-2007) as disclosed in the Influenza sequence database (www.flu.lanl.gov/). The NP consensus sequence can induce an immune response against an influenza virus. An exemplary sequence of the NP consensus comprises, consists essentially of, or consists of SEQ ID NO: 20.

NP Consensus Sequence
(SEQ ID NO: 20)

| | |
|---|---|
| 0 | MASQGTKRSY EQMETDGDRQ NATEIRASVG KMIDGIGRFY |
| | IQMCTELKLS |
| 50 | DHEGRLIQNS LTIEKMVLSA FDERRNKYLE EHPSAGKDPK |
| | KTGGPIYRRV |
| 100 | DGKWMRELVL YDKEEIRRIW RQANNGEDAT AGLTHIMIWH |
| | SNLNDATYQR |
| 150 | TRALVRTGMD PRMCSLMQGS TLPRRSGAAG AAVKGIGTMV |
| | MELIRMVKRG |
| 200 | INDRNFWRGE NGRKTRSAYE RMCNILKGKF QTAAQRAMVD |
| | QVRESRNPGN |
| 250 | AEIEDLIFLA RSALILRGSV AHKSCLPACA YGPAVSSGYD |
| | FEKEGYSLVG |
| 300 | IDPFKLLQNS QIYSLTRPNE NPAHKSQLVW MACHSAAFED |
| | LRLLSFIRGT |
| 350 | KVSPRGKLST RGVQIASNEN MDNMGSSTLE LRSGYWAIRT |
| | RSGGNTNQQR |
| 400 | ASAGQTSVQP TFSVQRNLPF EKSTIMAAFT GNTEGRTSDM |
| | RAEIIRMMEG |
| 450 | AKPEEVSFRG RGVFELSDEK ATNPIVPSFD MSNEGSYFFG |
| | DNAEEYDN |

In certain embodiments, the polypeptide encoded by the polynucleotide of the present invention further comprises a linker interposed between any two M2 ectodomain peptides. In one embodiment, any M2 ectodomain peptide present in the polypeptide has a linker peptide interposed between it and the adjacent M2 ectodomain peptide. In general, any given polypeptide may have no linker, one linker or multiple linker peptides. When more than one linker peptide is present in the polypeptides, the linker can be the same or different. The polypeptide can comprise at least two, at least three, at least four, at least five, at least six, at least seven, at least eight, at least nine, at least ten, or at least eleven linker peptides. The linker peptides can be at least three, at least four, at least five, at least six, at least seven, at least eight, at least nine, at least ten, at least 11, at least 12, at least 13, at least 14, or at least 15 amino acids. In one embodiment, the linker peptides are five amino acids in length. In another embodiment, the linker peptides have low or minimum immunogenicity, hydrophobicity or hydrophilicity to the host.

In other embodiments, the linker peptides have homology to an amino acid sequence found in a lower organism. In still other embodiments, the linker peptides have no homology to an amino acid sequence found in a primate. If these linker peptides are immunogenic and result in production of antibodies, those antibodies would circulate in the host with an opportunity to bind to any homologous antigens. If the potential antigens are self proteins, the immunogenicity is not desired. If the potential antigens are homologous to pathogens or other microbes, the immunogenicity may be beneficial.

In certain embodiments, the linker peptides used in the invention may be homologous to an amino acid sequence found in Burkholderia sp. H160, Arthrospira maxima CS-328, Helicoverpa armigera SNPV, Francisella novicida FTG, Peromyscus californicus insignis, Helicobacter pylori G27, Aliivibrio salmonicida LFI1238, Solanum pennellii, Saccharomyces cerevisiae AWRI1631, Cryptosporidium hominis, Bodo saltans, Nitrosococcus oceani C-27, beta-proteobacterium KB13, Campylobacterales bacterium GD 1, Candidatus Pelagibacter sp. HTCC7211, Thermodesulfovibrio yellowstonii DSM 11347, Bacillus cereus AH1134, Rhodobacterales bacterium Y4I, Leptospirillum sp. Group II ‘5-way CG’, Laccaria bicolor S238N-H82, Clostridium bartlettii DSM 16795, Claviceps purpurea, Tetraodon nigroviridis, Polynucleobacter necessarius STIR1, Piromyces rhizinflatus, neuraminidase [Influenza A virus (A/chicken/Iran/16/2000 (H9N2))], neuraminidase [Influenza A virus (A/mallard/MN/1/2000 (H5N2))], Escherichia coli O157:H7 str. EC4045, S1 glycoprotein [Infectious bronchitis virus], neuraminidase [Influenza A virus (A/Leiden/01272/2006 (H3N2))], neuraminidase [Influenza A virus (A/Tilburg/45223/2005 (H3N2))], neuraminidase [Influenza A virus (A/Pennsylvania/PIT25/2008 (H3N2))], neuraminidase [Influenza A virus (A/NYMC X-171A(Puerto Rico/8/1934-Brisbane/10/2007)(H3N2))], neuraminidase [Influenza A virus (A/Managua/26/2007 (H3N2))], neuraminidase [Influenza A virus (A/Hong Kong/1-1-MA-20D/1968 (H3N2))], neuraminidase [Influenza A virus (A/Czech Republic/1/1966 (H2N2))], neuraminidase [Influenza A virus (A/chicken/Shanghai/2/1999 (H9N2))], neuraminidase [Influenza B virus (B/Myanmar/M170/2007)], neuraminidase [Influenza A virus (A/Myanmar/M187/2007 (H3N2))], neuraminidase [Influenza A virus (A/Guinea fowl/New York/101276-1/2005 (H7N2))], neuraminidase [Influenza A virus (A/Muscovy duck/New York/87493-3/2005 (H7N2))], neuraminidase [Influenza A virus (A/turkey/New York/122501-2/2005 (H7N2))], and/or neuraminidase [Influenza A virus (A/mallard/Italy/4223-2/2006 (H5N2))]. In a specific embodiment, a linker peptide used in the present invention is Gly-Ser-Ala-Ser-Gly (GSASG) (SEQ ID NO: 21).

In other embodiments, the linker peptides have homology to an amino acid sequence found in Staphylococcus phage phi2958PVL, Streptomyces rimosus, Bodo saltans, Coprothermobacter proteolyticus DSM 5265, and/or Leptospirillum sp. Group II 5-way CG'. In a specific embodiment, a linker peptide used in the invention is Ser-Gly-Ser-Gly-Ala (SGSGA) (SEQ ID NO: 22).

In still other embodiments, the linker peptides have homology to an amino acid sequence found in Drosophila montana, polyprotein [Tomato torrado virus], immunoglobulin heavy chain variable region [Canis lupus familiaris], polyprotein [Dengue virus I], or oxysterol binding protein [Mus musculus]. In a particular embodiment, a linker peptide used in the invention is Ser-Ala-Gly-Ser-Gly (SAGSG) (SEQ ID NO: 23).

A linker peptide used in the present invention can be any linker known in the art, for example, an scFv linkers used for a single chain antibody, e.g., scFv. In one embodiment, the linker peptide is the sequence (Gly)ₙ. In another embodiment, the linker peptide comprises the sequence (GlyAla)ₙ. In other embodiments, the linker peptide comprises the sequence (GGS)ₙ, (EGGS)ₙ, (SEQ ID NO: 24) or (GGS)n(GGGGS)n (SEQ ID NO: 25), wherein n is an integer from 1-10, 5-20, 10-30, 20-50, 40-80, or 50-100.

The present invention also provides an isolated polynucleotide encoding a fusion protein comprising a coding region, which encodes a polypeptide comprising multiple copies of the M2 ectodomain peptides (METR), wherein the polypeptide further comprises one or more epitope. In one embodiment, the polypeptide further comprises at least one, at least two, at least three, at least four, at least five, at least six, at least seven, at least eight, at least nine, at least ten, at least eleven, or at least twelve epitopes. In another embodiment, the epitopes is at least four, at least five, at least six, at least seven, at least eight, at least nine, at least ten, at least 11, at least 12, at lesat 13, at least 14, at least 15, at least 16, at least 17, at least 18, at least 19, at least 20, at least 21, at least 22, at least 23, at least 24 or at least 25 amino acids in length. In one embodiment, any M2 ectodomain peptide present in the polypeptide has an epitope interposed between it and the adjacent M2 ectodomain peptide. In another embodiment, the polypeptides may further comprise one epitope or more than one epitopes interposed between the M2 ectodomains. When more than one epitope is present, the epitopes can be the same or different.

The epitope can be a B-cell epitope or a T-cell epitope. B-cell epitopes useful for the invention may be derived from the M2 protein, e.g., an antibody epitope located within the N-terminal 19-20 amino acids of the M2 ectodomain. Non limiting examples of the B-cell epitopes are amino acids 5-12 of SEQ ID NO: 1, amino acids 1-10 of SEQ ID NO: 1, amino acids 7-17 of SEQ ID NO: 2, amino acids 5-14 of SEQ ID NO: 3 or amino acids 3-19 of SEQ ID NO: 3. B-cell epitopes can be derived from other domains of the M2 protein, e.g., transmembrane domain or cytoplasmic domain. Alternatively, B-cell epitopes can be obtained from any influenza proteins or fragments thereof, e.g., hemagglutinin (HA), neuraminidase (NA), nucleoprotein (NP), Matrix 1 protein (M1), Matrix 2 protein (M2), non-structural protein (NS), RNA polymerase PA subunit (PA), RNA polymerase PB1 subunit (PB1), or RNA polymerase PB2 subunit (PB2).

T-cell epitopes used in the present invention can comprise any number of amino acids and be derived from any known antigens or immunogens. In one embodiment, T-cell epitopes can be derived from any influenza proteins or fragments thereof, e.g., hemagglutinin (HA), neuraminidase (NA), nucleoprotein (NP), Matrix 1 protein (M1), Matrix 2 protein (M2), non-structural protein (NS), RNA polymerase PA subunit (PA), RNA polymerase PB1 subunit (PB1), or RNA polymerase PB2 subunit (PB2). In a particular embodiment, T-helper cell epitopes can contain 9 core amino acids with 3 flanking amino acids on each side for a total of 15 amino acids. Its binding to the clefts of the Major Histocompatibility Complex (MHC in mice, HLA in humans) can be calculated by the known methods. The high-scoring peptides are predicted to be ligands for those MHC of HLA molecules.

In a specific embodiment, T-cell epitopes used in the invention comprises an amino acid sequence selected from the group consisting of SEQ ID NO: 26, SEQ ID NO: 27, or both, which are described in U.S. Pat. No. 6,663,871, incorporated herein by reference in its entirety.

The present invention includes multiple copies of M2 ectodomain peptides, one or more optional linker peptide interposed between two or more M2 ectodomain peptides, and/or one or more optional epitope interposed between two or more M2 ectodomain peptides. In one embodiment, the polynucleotides of the present invention encodes a polypeptide comprising at least six M2 ectodomain peptides (M2e#1, M2e#2, M2e#3, M2e#4, M2e#5, and M2e#6), one or more linker peptide interposed between any two or more ectodomain peptides (e.g., M2e#1-M2e#2, M2e#2-M2e#3, M2e#3-M2e#4, M2e#4-M2e#5, or M2e#5-M2e#6), and one or more epitopes interposed between any two or more M2 ectodomain peptides. In a specific embodiment, the polypeptide comprises six M2 ectodomain peptides, three linker peptides interposed there between, and two epitopes interposed there between, wherein:
(1) the first linker peptide is interposed between the first M2 ectodomain peptide (M2e#1) and the first M2 ectodomain peptide (M2e#2);
(2) the second linker peptide is interposed between the second M2 ectodomain peptide (M2e#2) and the third M2 ectodomain peptide (M2e#3);
(3) the third linker peptide is interposed between the third M2 ectodomain peptide (M2e#3) and the fourth M2 ectodomain peptide (M2e#4);
(4) the first epitope is interposed between the fourth M2 ectodomain peptide (M2e#4) and the fifth M2 ectodomain peptide (M2e#5); and
(5) the second epitope is interposed between the fifth M2 ectodomain peptide (M2e#5) and the sixth M2 ectodomain peptide (M2e#6).

In some embodiments, an isolated polynucleotide of the instant invention comprises a coding region, which encodes a polypeptide comprising multiple copies of the M2 ectodomain peptides, wherein the coding region further comprises an additional nucleic acid sequence. The additional nucleic acid sequence can, in certain embodiments, encode an additional polypeptide, optionally fused to the polypeptide of the invention. The additional polypeptide can comprise at least one immunogenic epitope of an influenza virus, wherein the epitope elicits a B-cell (antibody) response, a T-cell response, or both.

Various additional nucleic acids can be used to encode their respective additional polypeptides. In one embodiment, the additional polypeptide is fused to the METR polypeptide of the present invention. In another embodiment, the additional polypeptide is not fused to the METR polypeptide of the present invention but is produced in the same vector expressing the METR polypeptide. Non-limiting examples of the additional nucleic acid sequence are nucleic acid sequences encoding an influenza protein, variant, derivative, analogue, or fragment thereof. The influenza protein can be selected from the group consisting of hemagglutinin (1-1A), neuraminidase (NA), nucleoprotein (NP), Matrix 1 protein (M1), Matrix 2 protein (M2), non-structural protein (NS), RNA polymerase PA subunit (PA), RNA polymerase PB1 subunit (PB1), or RNA polymerase PB2 subunit (PB2).

In some embodiments, a additional polypeptide is selected from the group consisting of an N- or C-terminal peptide imparting stabilization, secretion, or simplified purification, i.e., His-tag, ubiquitin tag, NusA tag, chitin binding domain, ompT, oinpA, pelB, DsbA, DsbC, c-myc, KSI, polyaspartic acid, (Ala-Trp-Trp-Pro)n (SEQ ID NO: 28), polypheriyalanine, polycysteine, polyarginine, B-tag, HSB-tag, green fluorescent protein (GFP), hemagglutinin influenza virus (HAI), calmodulin binding protein (CBP), galactose-binding protein, maltose binding protein (MBP), cellulose binding domains (CBD's), dihydrofolate reductase (DHFR), glutathione-S-transferase (GST), streptococcal protein G, staphylococcal protein A, T7gene10, avidin/streptavidin/Strep-tag, trpE, chloramphenicol acetyltransferase, lacZ (β-Galactosidase), His-patch thioredoxin, thioredoxin, FLAG™ peptide (Sigma-Aldrich), S-tag, and T7-tag. See e.g., Stevens, R. C., Structure, 8:R177-R185 (2000). The heterologous polypeptides can further include any pre- and/or pro-sequences that facilitate the transport, translocations, processing and/or expression of the METR sequences or any useful immunogenic sequence, including but not limited to sequences that encode a T-cell epitope of a microbial pathogen, or other immunogenic proteins and/or epitopes. Other suitable additional polypeptides can include a leader sequence or signal sequence.

### Codon Optimization

Also included within the scope of the invention is a codon-optimized polynucleotide encoding a polypeptide comprising multiple copies of M2 ectodomain peptide sequences. Modifications of nucleic acids encoding the polypeptide can readily be accomplished by those skilled in the art, for example, by oligonucleotide-directed site-specific mutagenesis of a polynucleotide coding for a polypeptide. Such modified polypeptide can be encoded by a codon-optimized nucleotide sequence. Such modifications impart one or more amino acid substitutions, insertions, deletions, and/or modifications to expressed polypeptides including fragments, variants, and derivatives. Such modifications may enhance the immunogenicity of antigens, for example, by increasing cellular immune responses compared with unmodified polypeptides. Such modification may enhance solubility of the polypeptides. Alternatively, such modifications may have no effect. For example, an M2 ectodomain peptide may be modified by introduction, deletion, or modification of particular cleavage sites for proteolytic enzymes active in antigen presenting cells, to enhance immune responses to particular epitopes.

As appreciated by one of ordinary skill in the art, various nucleic acid coding regions will encode the same polypeptide due to the redundancy of the genetic code. Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 3. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the polypeptides encoded by the DNA.

TABLE 3
The Standard Genetic Code

| | T | C | A | G |
|---|---|---|---|---|
| T | TTT Phe (F) | TCT Ser (S) | TAT Tyr (Y) | TGT Cys |
| | | | | (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp |
| | | | | (W) |
| C | CTT Leu (L) | CCT Pro (P) | CAT His (H) | CGT Arg |
| | | | | (R) |
| | CTC Leu (L) | CCC Pro (P) | CAC His (H) | CGC Arg |
| | | | | (R) |
| | CTA Leu (L) | CCA Pro (P) | CAA Gln (Q) | CGA Arg |
| | | | | (R) |
| | CTG Leu (L) | CCG Pro (P) | CAG Gln (Q) | CGG Arg |
| | | | | (R) |
| A | ATT Ile (I) | ACT Thr (T) | AAT Asn (N) | AGT Ser |
| | | | | (S) |
| | ATC Ile (I) | ACC Thr (T) | AAC Asn (N) | AGC Ser |
| | | | | (S) |
| | ATA Ile (I) | ACA Thr (T) | AAA Lys (K) | AGA Arg |
| | | | | (R) |
| | ATG Met (M) | ACG Thr (T) | AAG Lys (K) | AGG Arg |
| | | | | (R) |
| G | GTT Val (V) | GCT Ala (A) | GAT Asp (D) | GGT Gly |
| | | | | (G) |
| | GTC Val (V) | GCC Ala (A) | GAC Asp (D) | GGC Gly |
| | | | | (G) |
| | GTA Val (V) | GCA Ala (A) | GAA Glu (E) | GGA Gly |
| | | | | (G) |
| | GTG Val (V) | GCG Ala (A) | GAG Glu (E) | GGG Gly |
| | | | | (G) |

It is to be appreciated that any polynucleotide that encodes a polypeptide in accordance with the invention falls within the scope of this invention, regardless of the codons used.

Many organisms display a bias for use of particular codons to code for insertion of a particular amino acid in a growing polypeptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The present invention provides an isolated polynucleotide containing a polynucleotide comprising, consisting essentially of, or consisting of a coding optimized coding region which encodes an influenza protein, e.g., METR, disclosed herein. In such embodiments the codon usage is adapted for optimized expression in the cells of a given prokaryote or eukaryote.

The polynucleotides are prepared by incorporating codons preferred for use in the genes of a given species into the DNA sequence. Also provided are polynucleotide expression constructs, vectors, and host cells comprising nucleic acid fragments of codon-optimized coding regions which encode the influenza polypeptide, e.g., METR, as well as various methods of using the polynucleotide expression constructs, vectors, and host cells to treat or prevent influenza infections in an animal.

Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/ (visited May 30, 2006), and these tables can be adapted in a number of ways. See Nakamura, Y., et al., “Codon usage tabulated from the international DNA sequence databases: status for the year 2000” Nucl. Acids Res. 28:292 (2000). A codon usage table for human calculated from GenBank Release 151.0, is reproduced below as Table 4 (from www.kazusa.or.jp/codon/ supra). These tables use mRNA nomenclature, and so instead of thymine (T) which is found in DNA, the tables use uracil (U) which is found in RNA. The tables have been adapted so that frequencies are calculated for each amino acid, rather than for all 64 codons.

TABLE 4
Codon Usage Table for Human Genes (Homo
sapiens)

| Amino | | Frequency |
|---|---|---|
| Acid | Codon | of Usage |
| Phe | UUU | 0.4525 |
| | UUC | 0.5475 |
| Leu | UUA | 0.0728 |
| | UUG | 0.1266 |
| | CUU | 0.1287 |
| | CUC | 0.1956 |
| | CUA | 0.0700 |
| | CUG | 0.4062 |
| Ile | AUU | 0.3554 |
| | AUC | 0.4850 |
| | AUA | 0.1596 |
| Met | AUG | 1.0000 |
| Val | GUU | 0.1773 |
| | GUC | 0.2380 |
| | GUA | 0.1137 |
| | GUG | 0.4710 |
| Ser | UCU | 0.1840 |
| | UCC | 0.2191 |
| | UCA | 0.1472 |
| | UCG | 0.0565 |
| | AGU | 0.1499 |
| | AGC | 0.2433 |
| Pro | CCU | 0.2834 |
| | CCC | 0.3281 |
| | CCA | 0.2736 |
| | CCG | 0.1149 |
| Thr | ACU | 0.2419 |
| | ACC | 0.3624 |
| | ACA | 0.2787 |
| | ACG | 0.1171 |
| Ala | GCU | 0.2637 |
| | GCC | 0.4037 |
| | GCA | 0.2255 |
| | GCG | 0.1071 |
| Tyr | UAU | 0.4347 |
| | UAC | 0.5653 |
| His | CAU | 0.4113 |
| | CAC | 0.5887 |
| Gln | CAA | 0.2541 |
| | CAG | 0.7459 |
| Asn | AAU | 0.4614 |
| | AAC | 0.5386 |
| Lys | AAA | 0.4212 |
| | AAG | 0.5788 |
| Asp | GAU | 0.4613 |
| | GAC | 0.5387 |
| Glu | GAA | 0.4161 |
| | GAG | 0.5839 |
| Cys | UGU | 0.4468 |
| | UGC | 0.5532 |
| Trp | UGG | 1.0000 |
| Arg | CGU | 0.0830 |
| | CGC | 0.1927 |
| | CGA | 0.1120 |
| | CGG | 0.2092 |
| | AGA | 0.2021 |
| | AGG | 0.2011 |
| Gly | GGU | 0.1632 |
| | GGC | 0.3438 |
| | GGA | 0.2459 |
| | GGG | 0.2471 |

By utilizing these or similar tables, one of ordinary skill in the art can apply the frequencies to any given polypeptide sequence, and produce a nucleic acid fragment of a codon-optimized coding region which encodes the polypeptide, but which uses codons optimal for a given species.

A number of options are available for synthesizing codon-optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

### Vectors

The present invention relates to vectors, e.g., plasmids, cosmids, viruses, and bacteriophages, used conventionally in genetic engineering, the vectors comprising a polynucleotide encoding the influenza antigen or the polypeptide comprising multiple copies of the M2 ectodomain peptides, e.g., METR, which are arranged in any order relative to each other.

In one embodiment, the vector is an expression vector and/or a gene transfer or targeting vector. In another embodiment, the vector is a viral vector. Expression vectors derived from viruses such as retroviruses, vaccinia viruses, adeno-associated viruses, adeno viruses, herpes viruses, or bovine papilloma viruses, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells. The vectors containing the polynucleotides of the invention (e.g., the multiple copies of the M2 ectodomain sequences (METR)) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra. In general, vectors compatible with the instant invention will comprise a selection marker, appropriate restriction sites to facilitate cloning of the desired gene and the ability to enter and/or replicate in eukaryotic or prokaryotic cells.

In certain embodiments, the present invention is directed to a poxvirus, e.g., a vaccinia virus, e.g., a modified vaccinia virus Ankara (MVA), comprising a polynucleotide, which encodes a polypeptide comprising an influenza polypeptide, e.g., the multiple copies of the M2 ectodomain (METR), NP, or both. MVA is a highly attenuated vaccinia virus strain, a member of the genus Orthopoxvirus in the family of Poxyiridae. Poxviruses include four genera of pox viruses, i.e., orthopox, parapox, yatapox, and molluscipox viruses. Orthopox viruses include without limitation, variola virus (the agent causing smallpox), vaccinia virus, cowpox virus, monkeypox virus, and raccoon poxvirus; Parapox viruses include, without limitation, orf virus, pseudocowpox, and bovine papular stomatitis virus; Yatapox viruses include, without limitation, tanapox virus and yaba monkey tumor virus; and Molluscipox viruses include molluscum contagiosum virus (MCV).

Vaccinia viruses have been used as a live vaccine to immunize against the human smallpox disease or to engineer viral vectors for recombinant gene expression or for the potential use as recombinant live vaccines (Mackett, M. et al., 1982 PNAS USA 79:7415-7419; Smith, G. L. et al., 1984 Biotech Genet Engin Rev 2:383-407). The engineered viral vectors may contain DNA sequences (genes) which code for foreign antigens, e.g., Influenza polypeptides, with the aid of DNA recombination techniques. If the gene is integrated at a site in the viral genome non-essential for the life cycle of the virus, the newly produced recombinant vaccinia virus incorporating the foreign gene may be capable of infecting host cells and thus inducing expression of the foreign protein in the host cells. (U.S. Pat. Nos. 5,110,587; 83,286; and 110,385, incorporated herein by reference in their entireties). Recombinant vaccinia viruses (e.g., MVA) prepared in this way are used according to this invention as live vaccines for the prophylaxis of infectious Influenza diseases in vivo.

In one embodiment, an example of vaccinia virus strains used herein is a highly attenuated modified vaccinia virus Ankara (MVA). MVA was generated by long-term serial passages of the Ankara strain of vaccinia virus (CVA) on chicken embryo fibroblasts (for review see Mayr, A. et al. 1975 Infection 3:6-14; Swiss Patent No. 568,392). MVA viruses are publicly available, e.g., from the American Type Culture Collection as ATCC No.: VR-1508. MVA is distinguished by its attenuation, e.g., diminished virulence and limited ability to reproduce infectious virions in certain mammalian cells, while maintaining good immunogenicity and full capacity to replicate and produce infectious virions in avian cells. The MVA virus has alterations in its genome that induce attenuation relative to the parental CVA strain. Six major deletions of genomic DNA (deletion I, II, III, IV, V, and VI) totaling 31,000 base pairs (about 10% of its genome) have been identified (Meyer, H. et al. 1991 J Gen Virol 72:1031-1038, which is incorporated herein by reference in its entirety). The resulting MVA virus became severely attenuated in mammalian cells. Due to its attenuation, MVA of the present invention may be avirulent even in immunosuppressed individuals and have very little side effects associated with the use of MVA in a live vaccine against an infectious influenza disease.

The vectors, e.g., MVAs of the present invention may undergo limited replication in human cells as its replication is blocked in the late stage of infection. The limited replication prevents the assembly to mature infectious virions. Nevertheless, the vectors, e.g., MVAs of the present invention are capable of expressing viral and recombinant genes at high levels even in non-permissive cells and also capable of serving as an efficient and safe gene expression vector.

In one embodiment of the present invention, the isolated polynucleotide sequence coding for the influenza polypeptides, e.g., METR, is fused to MVA flanking sequences adjacent to a naturally occurring deletion, e.g., deletion I, deletion II, deletion III, deletion IV, deletion, V, or deletion VI, or other non-essential sites present in the MVA genome at the 5′ or 3′ end of the polynucleotide. The non-essential regions of the MVA genome include, but are not limited to, intergenic regions and naturally occurring deletion regions as well as other genes that are not required for replication, e.g., the tk gene. The DNA sequence carrying the polynucleotide sequence which codes for one or more Influenza antigens, e.g., METR, can be linear or circular, being a polymerase chain reaction product or plasmid, and may further comprise a regulatory sequences such as a promoter which is operatively associated to the coding region encoding at least one influenza polypeptide. Non-limiting examples of the regulatory elements include the vaccinia 11 ka gene as are described in EP-A-198,328, and those of the 7.5 kDa gene (EP-A-110,385), each of which is incorporated herein by reference in its entirety.

In some embodiments, the present invention provides a recombinant MVA containing a polynucleotide that comprises a promoter operably associated with the coding sequence encoding the influenza polypeptide antigen, e.g., METR. In a particular embodiment, the promoter is a viral promoter (e.g., a vaccinia virus or Modified Vaccinia Ankara Virus promoter). In a further particular embodiment, the promoter is a synthetic promoter. In further embodiments, the promoter is a strong promoter. In a specific embodiment, the promoter is a strong synthetic promoter. In a specific embodiment, the promoter is the PS promoter having the sequence AAAAATTGAAATTTTATTTTTTTTTTTTGGAATATAAATA (SEQ ID NO: 29) (Chakrabarti, Sisler and Moss (1997). Biotechniques 23:1094-1097). In a further specific embodiment the promoter is the modified 115 promoter having the sequence AAAAAATGAAAATAAATACAAAGGTTCTTGAGGGTTGTGTTAAATTGAAA GCGAGAAATAATCATAAATT (SEQ ID NO: 30) (Rosel et al. (1986). J. Virol. 60 (2): 236-249).

Poxvirus transcriptional control regions comprise a promoter and a transcription termination signal. Gene expression in poxviruses is temporally regulated, and promoters for early, intermediate, and late genes possess varying structures. Certain poxvirus genes are expressed constitutively, and promoters for these “early-late” genes bear hybrid structures. Synthetic early-late promoters have also been developed. See Hammond J. M., et al., J. Virol. Methods 66:135-8 (1997); Chakrabarti S., et al., Biotechniques 23:1094-7 (1997). Therefore, in the present invention, any poxvirus promoter may be used, e.g., early, late, or constitutive promoters.

Non-limiting examples of early promoters include the 7.5-kD promoter (also a late promoter), the DNA pol promoter, the tk promoter, the RNA pol promoter, the 19-kD promoter, the 22-kD promoter, the 42-kD promoter, the 37-1d) promoter, the 87-kD promoter, the H3′ promoter, the H6 promoter, the D1 promoter, the D4 promoter, the D5 promoter, the D9 promoter, the D12 promoter, the 13 promoter, the M1 promoter, and the N2 promoter. See, e.g., Moss, B., “Poxyiridae and their Replication” IN Virology, 2d Edition, B. N. Fields, D. M. Knipe et al., Eds., Raven Press, p. 2088 (1990). Early genes transcribed in vaccinia virus and other poxviruses recognize the transcription termination signal TTTTTNT (SEQ ID NO: 31), where N can be any nucleotide. Transcription normally terminates approximately 50 bp upstream of this signal. Accordingly, if heterologous genes are to be expressed from poxvirus early promoters, care must be taken to eliminate occurrences of this signal in the coding regions for those genes. See, e.g., Earl, P. L., et al., J. Virol. 64:2448-51 (1990).

Examples of late promoters include without limitation the 7.5-1d) promoter, the MIL promoter, the 37-kD promoter, the 11-kD promoter, the 11L promoter, the 12L promoter, the 13L promoter, the 15L promoter, the 17L promoter, the 28-kD promoter, the H1L promoter, the H3L promoter, the H5L promoter, the H6L promoter, the H8L promoter, the D11L promoter, the D12L promoter, the D13L promoter, the A1l promoter, the A2L promoter, the A3L promoter, and the P4b promoter. See, e.g., Moss, B., “Poxyiridae and their Replication” IN Virology, 2d Edition, B. N. Fields, D. M. Knipe et al., Eds., Raven Press, p. 2090 (1990). The late promoters apparently do not recognize the transcription termination signal recognized by early promoters.

Non-limiting examples of constitutive promoters for use in the present invention include the synthetic early-late promoters described by Hammond and Chakrabarti, the MH-5 early-late promoter, and the 7.5-kD or “p7.5” promoter.

The present invention is also directed to a vector, e.g., an MVA, comprising the polynucleotide of the invention, and an additional nucleic acid sequence. The additional nucleic acid sequence can be inserted in an insertion site that is same as or different from the site in which the polynucleotide sequence encoding the multiple copies of the M2 ectodomain peptides (METR) is inserted. The additional nucleic acid sequence can comprise a coding region encoding an additional polypeptide. The additional nucleic acid sequence can further be connected to a promoter. In one embodiment, the vector of the invention, e.g., MVA, encodes two or more antigens or immunogens, one antigen being METR or NP consensus and another antigen being an additional polypeptide. The additional polypeptide can be an influenza protein, variant, fragment, derivative, or analogue thereof as used herein.

In a particular embodiment, the vector of the invention, e.g., MVA, expresses at least two influenza antigens, e.g., METR and HA, METR and NP, HA and NP, and at least three influenza antigens, e.g., METR, HA, and NP. In certain embodiments, a vector of the invention express at least four, at least five, at least six, at least seven, or at least eight influenza antigens.

The present invention also provides a method of producing a vector, e.g., an MVA comprising introducing into a host cell infected with a vector, e.g., an MVA, an isolated polynucleotide (DNA) construct comprising a polynucleotide encoding the multiple copies of the M2 ectodomain peptides (METR) to allow homologous recombination. Once the DNA construct encoding the METR polypeptide is introduced into the host cell and the foreign DNA sequence is recombined with the viral DNA, the resulting recombinant MVA virus comprises a polynucleotide encoding the METR polypeptide. The present invention also provides isolating the resulting MVA by known techniques, e.g., with the aid of a marker. The DNA construct carrying an METR polypeptide genes may also be introduced into the MVA infected cells by transfection, for example by means of calcium phosphate precipitation (Graham et al. 1973 Virol 52:456-467; Wigler et al. 1979 Cell 16:777-785), by means of electroporation (Neumann et al. 1982 EMBO J. 1:841-845), by microinjection (Graessmann et al. 1983 Meth Enzymol 101:482-492), by means of liposomes (Straubinger et al. 1983 Meth Enzymol 101:512-527), by means of spheroplasts (Schaffher 1980 PNAS USA 77:2163-2167) or by other methods known to those skilled in the art. The references listed herein for the transfection methods are incorporate herein by reference in their entireties.

According to the present invention, the recombinant MVA vaccinia viruses can be isolated by several well-known techniques, for example the K1L-gene based selection protocol. As a non-limiting example, a DNA-construct may contain a DNA-sequence which codes for the Vaccinia Virus K1L protein or a K1L-derived polypeptide as a marker and a DNA sequence encoding the METR polypeptide both flanked by DNA sequences flanking a non-essential site, e.g. a naturally occurring deletion, e.g. deletion III, within the MVA genome.

Host cells used for the present invention include, but are not limited to, eukaryotic cells, avian cells, mammalian cells, or human cells. Non-limiting examples of eukaryotic cells are BHK-21 (ATCC CCL-10), BSC-1 (ATCC CCL-26), CV-1 (ECACC 87032605) or MA104 (ECACC 85102918). In one embodiment, host cells are avian cells including, but not limited to, chicken cells, duck cells, or quail cells. Non-limiting examples of avian cells are chicken fibroblast cells, quail fibroblast cells, QT9 cells, QT6 cells, QT35 cells, Vero cells, MRC-5 cells, chicken embryo derived LSCC-H32 cells, chicken DF-1 cells, or primary chicken embryo fibroblast (CEF) cells. In other embodiments, the avian cells used as host cells in the invention are immortalized. The immortalized avian cells may be immortalized duck cells, including but not limited to AGE1cr cells and AGE1cr.pIX cells described in US Application Publication Nos. US 2008/0227146 A1 and International Publication No. WO 2007/054516 A1, incorporated herein by reference in their entireties. Other immortalized duck cells useful in the present invention include embryonic derived stem cells, e.g., EB66′ cells, described in US Application Publication No. US 2010/062489 A1, which is incorporated herein by reference in its entirety. Other useful avian cell lines are described in PCT Application Publication No. WO 2006/1088646 A2, US Application Publication No. 2006/0233834 A1, and U.S. Pat. Nos. 5,830,510 and 6,500,668, all of which are incorporated herein by reference in their entireties.

### Polypeptides

The present invention also includes a polypeptide comprising multiple copies of the M2 ectodomain peptides (METR). In one embodiment, the present invention is an isolated polypeptide comprising at least five of the following influenza virus Matrix 2 protein ectodomain peptides arranged in any order respective to each other: (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6 S).

In one embodiment, the present invention provides an isolated polypeptide comprising at least three of the following M2 ectodomain peptides arranged in any order respective to each other: (i) SEQ ID NO: 1 (M2e#1C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S). In another embodiment, the polypeptide comprises at least four, five, six, seven, eight, nine, ten, eleven, or twelve M2 ectodomain peptides. In some embodiments, the polypeptide of the invention is a fusion protein and induces an immune response against influenza viruses. In certain embodiments, the polypeptide of the invention comprises at least three of the following M2 ectodomain peptides arranged in any order respective to each other: (a) (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); and (vi) SEQ ID NO: 6 (M2e#6_C) or (b) (i) SEQ ID NO: 7 (M2e#1_S); (ii) SEQ ID NO: 8 (M2e#2_S); (iii) SEQ ID NO: 9 (M2e#3_S); (iv) SEQ ID NO: 10 (M2e#4_S); (v) SEQ ID NO: 11 (M2e#5_S); and (vi) SEQ ID NO: 12 (M2e#6_S).

In a particular embodiment, the polypeptide of the present invention comprises the following six amino acid sequences arranged in any order respective to each other: (a) (i) SEQ ID NO: 1 (M2e#1_C); (ii) SEQ ID NO: 2 (M2e#2_C); (iii) SEQ ID NO: 3 (M2e#3_C); (iv) SEQ ID NO: 4 (M2e#4_C); (v) SEQ ID NO: 5 (M2e#5_C); and (vi) SEQ ID NO: 6 (M2e#6_C); or (b) (i) SEQ ID NO: 7 (M2e#1_S); (ii) SEQ ID NO: 8 (M2e#2_S); (iii) SEQ ID NO: 9 (M2e#3_S); (iv) SEQ ID NO: 10

(M2e#4_S); (v) SEQ ID NO: 11 (M2e#5_S); and (vi) SEQ ID NO: 12 (M2e#6_S). In other embodiments, a polypeptide of the invention comprises the NP consensus sequence of SEQ ID NO: 20.

A polypeptide of the present invention can be a fusion protein, which further comprises an additional polypeptide. Non-limiting examples of the additional polypeptide are additional influenza polypeptides, variants, derivatives, analogues, or fragments thereof. Additional polypeptides may be immunogenic or antigenic and can be any known antigens.

### Compositions

Compositions, e.g., pharmaceutical or vaccine compositions that contain an immunologically effective amount of an isolated polynucleotide, polypeptide, or vector, e.g., MVA, are further embodiments of the invention. Such compositions may include, for example, lipopeptides (e.g., Vitiello, A. et al., J. Clin. invest. 95:341, 1995), polypeptides encapsulated, e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995); polypeptide compositions contained in immune stimulating complexes (ISCOMS) (see, e.g., Takahashi et al., Nature 344:873-875, 1990; Hu, et al., Clin Exp Immunol. 113:235-243, 1998); multiple antigen peptide systems (MAPS) (see e.g., Tam, J. P., Proc. Natl. Acad. Sci. U.S.A. 85:5409-5413, 1988; Tam, J. P., J. Immunol. Methods 196:17-32, 1996); particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995); adjuvants (e.g., incomplete Freund's adjuvant) (Warren, H. S., Vogel, F. R., and Chedid, L. A. Annu. Rev. Immunol. 4:369, 1986; Gupta, R. K. et al., Vaccine 11:293, 1993); or liposomes (Reddy, R. et al., J. Immunol. 148:1585, 1992; Rock, K. L., Immunol. Today 17:131, 1996). The compositions can be pharmaceutical, antigenic, immunogenic, or vaccine compositions.

Compositions, e.g., vaccine compositions, of the present invention can be formulated according to the known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995), both of which are incorporated herein by reference in their entireties. Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives. Once formulated, the compositions of the invention can be administered directly to the subject. The subjects to be treated can be animals; in particular, human.

The concentration of polynucleotides, polypeptides, or vectors, e.g., MVA, in the compositions of the invention can vary widely, i.e., from less than about 0.1%, usually at or at least about 2% to as much as 20% to 50% or more by weight, and will be selected primarily by fluid volumes, viscosities, etc., in accordance with the particular mode of administration selected.

In one embodiment, a composition of the present invention comprises an isolated polynucleotide comprising a coding sequence, which encodes a polypeptide comprising multiple copies, e.g., at least three, four, five, six, seven, eight, nine, ten, eleven, or twelve of the following M2 ectodomain peptides arranged in any order respective to each other: (i) SEQ ID NO: 1 (M2e#1_C); (ii) SEQ ID NO: 2 (M2e#2S); (iii) SEQ ID NO: 3 (M2e#3_C); (iv) SEQ ID NO: 4 (M2e#4_C); (v) SEQ ID NO: 5 (M2e#5_C); (vi) SEQ ID NO: 6 (M2e#6_C); (vii) SEQ ID NO: 7 (M2e#1_S); (viii) SEQ ID NO: 8 (M2e#2_S); (ix) SEQ ID NO: 9 (M2e#3_S); (x) SEQ ID NO: 10 (M2e#4_S); (xi) SEQ ID NO: 11 (M2e#5_S); and (xii) SEQ ID NO: 12 (M2e#6_S). In another embodiment, a composition, e.g., a vaccine composition of the present invention comprises one or more vector, e.g., MVA, comprising a polynucleotide, which encodes multiple copies of the M2 ectodomain peptides (METR) or the NP consensus sequence. In some embodiments, a composition comprises a polypeptide of the present invention comprising multiple copies of the M2 ectodomain peptides.

In some embodiments, a host cell having a vector comprising the polynucleotide of the present invention is incorporated in a composition, as described in Eko, et al., J. Immunol., 173:3375-3382, 2004.

Certain compositions can further include one or more adjuvants before, after, or concurrently with the polynucleotide, polypeptide, or vector, e.g., MVA. A great variety of materials have been shown to have adjuvant activity through a variety of mechanisms. Potential adjuvants which may be screened for their ability to enhance the immune response according to the present invention include, but are not limited to: inert carriers, such as alum, bentonite, latex, and acrylic particles; pluronic block polymers, such as TITERMAX® (block copolymer CRL-8941, squalene (a metabolizable oil) and a microparticulate silica stabilizer), depot formers, such as Freund's adjuvant, surface active materials, such as saponin, lysolecithin, retinal, Quil A, liposomes, and pluronic polymer formulations; macrophage stimulators, such as bacterial lipopolysaccharide; polycationic polymers such as chitosan; alternate pathway complement activators, such as insulin, zymosan, endotoxin, and levamisole; and non-ionic surfactants, such as poloxamers, poly(oxyethylene)-poly(oxypropylene) tri-block copolymers, cytokines and growth factors; bacterial components (e.g., endotoxins, in particular superantigens, exotoxins and cell wall components); aluminum-based salts such as aluminum hydroxide; calcium-based salts; silica; polynucleotides; toxoids; serum proteins, viruses and virally-derived materials, poisons, venoms, imidazoquiniline compounds, poloxamers, mLT, and cationic lipids. International Patent Application, PCT/US95/09005 incorporated herein by reference describes use of a mutated form of heat labile toxin of enterotoxigenic E. coli (“mLT”) as an adjuvant. U.S. Pat. No. 5,057,540, incorporated herein by reference, describes the adjuvant, Qs21. In some embodiments, the adjuvant is a toll-like receptor (TLR) stimulating adjuvant. See e.g., Science 312:184-187 (2006). TLR adjuvants include compounds that stimulate the TLRs (e.g., TLR1-TLR17), resulting in an increased immune system response to the vaccine composition of the present invention. TLR adjuvants include, but are not limited to CpG (Coley Pharmaceutical Group Inc.) and MPL (Corixa). One example of a CpG adjuvant is CpG7909, described in WO 98/018810, US Patent Application Publication No. 2002/0164341A, U.S. Pat. No. 6,727,230, and International Publication No. WO98/32462, which are incorporated herein by reference in their entireties.

Dosages of the adjuvants can vary according to the specific adjuvants. For example, in some aspects, dosage ranges can include: 10 μg/dose to 500 μg/dose, or 50 μg/dose to 200 μg/dose for CpG. Dosage ranges can include: 2 μg/dose to 100 μg/dose, or 10 μg/dose to 30 μg/dose for MPL. Dosage ranges can include: 10 μg/dose to 500 μg/dose, or 50 μg/dose to 100 μg/dose for aluminum hydroxide. In a prime-boost regimen, as described elsewhere herein, an adjuvant may be used with either the priming immunization, the booster immunization, or both.

In certain adjuvant compositions, the adjuvant is a cytokine. Certain compositions of the present invention comprise one or more cytokines, chemokines, or compounds that induce the production of cytokines and chemokines, or a polynucleotide encoding one or more cytokines, chemokines, or compounds that induce the production of cytokines and chemokines. Examples of cytokines include, but are not limited to granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interferon alpha (MN), interferon beta (IFN), interferon gamma (IFN), interferon omega (IFN), interferon tau (IFN), interferon gamma inducing factor I (IGIF), transforming growth factor beta (TGF-), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th₂ response into a primarily cellular, or Th₁ response. Immune responses to a given antigen may be tested by various immunoassays well known to those of ordinary skill in the art, and/or described elsewhere herein.

Furthermore, the multiple copies of the M2 ectodomain peptides (METR) polypeptides may be conjugated to a bacterial toxoid, such as a toxoid from diphtheria, tetanus, cholera, H. pylori, or other pathogen. Furthermore, the METR polypeptide may be conjugated to a bacterial polysaccharide, such as the capsular polysaccharide from Neisseria spp., Streptococcus pneumoniae spp. or Haemophilus influenzae type-b bacteria.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more polypeptides of the invention, often at a concentration of 25%-75%.

In some embodiments, the present invention is directed to a multivalent vaccine. For example, a multivalent vaccine of the present invention can comprise a polynucleotide, polypeptide, or vector, e.g., MVA, wherein the polynucleotide or vector, e.g., MVA, encodes two or more influenza epitopes or the polypeptide comprises two or more influenza epitopes, when administered to a subject in need thereof in a sufficient amount. Two or more influenza epitopes may be derived from the same or different antigens. In certain embodiments, the multivalent vaccine induces an immune response against the influenza matrix 2 protein and an additional influenza protein or fragment thereof. In a particular embodiment, a composition of the present invention includes two or more polynucleotides, polypeptides, or vectors, e.g., MVAs, of the present invention. As a specific example, the present invention can include a composition comprising two or more populations of MVAs, wherein the first MVA comprises a polynucleotide comprising a coding region, which encodes multiple copies of the M2 ectodomain peptides (METR) of the present invention and the second MVA comprises a polynucleotide encoding an additional antigen, e.g., an additional influenza virus protein or fragment thereof. In one embodiment, the additional antigen is HA, NP consensus, variants, derivatives, analogues, or fragments thereof. In another embodiment, the additional antigen in the second MVA is a polypeptide comprising multiple copies of the M2e ectodomains that is not identical to the METR sequence expressed in the first MVA.

In certain embodiments, a multivalent vaccine composition of the instant comprises the polynucleotide, polypeptide, or vector, e.g., MVA, wherein the polynucleotide, polypeptide, or vector, e.g., MVA, when administered to a subject in need thereof in a sufficient amount, induces an immune response against an influenza virus and a polypeptide that elicits an immune reaction to one or more additional organisms and/or viruses, e.g., Haemophilus influenzae type b, Hepatitis B virus, Hepatitis A virus, Hepatitis C virus, Corynebacterium diphtheriae, Clostridium tetani, Polio virus, Rubeola virus, Rubella virus, myxovirus, Neisseria, e.g., N. gonnorrheae, Haemophilus ducrey, Granuloma inguinale, Calymmatobacterium granulomatis, human papilloma virus (HPV) type I and II, Ureaplasma urealyticutn, Mycoplasma hominis, Treponema pallidum, Poxvirus of the Molluscipox virus genus, Human Immunodeficiency Virus (HIV), Epstein-Barr virus (EBV), herpes simplex virus, or varicella-zoster virus.

The multivalent vaccine of the present invention can comprise a polynucleotide, polypeptide, or vector, e.g., MVA, and a compatible vaccine, wherein both the vaccine of the present invention and the compatible vaccine are targeted for a similar patient population, e.g., an immunocompromised population, children, infants, or elderly.

### Methods of Treatment/Prevention and Regimens

Also provided is a method to treat or prevent an influenza virus infection or a condition associated with an influenza virus infection in a subject comprising: administering to the subject in need thereof a composition containing the polynucleotide, polypeptide, or vector, e.g., MVA, of the present invention. In certain embodiments, the subject is a vertebrate, e.g., a mammal, e.g., a primate, e.g., a human. In some embodiments, the invention is directed to a method of inducing an immune response against an influenza virus in a subject, e.g., a host animal comprising administering an effective amount a composition containing any one or more of the polynucleotide, polypeptide, or vector, e.g., MVAs of the present invention.

In some embodiments, an animal can be treated with the polynucleotides, polypeptides, vectors, e.g., MVAs, or compositions prophylactically, e.g., as a prophylactic vaccine, to establish or enhance immunity to one or more influenza virus species in a healthy animal prior to exposure to an influenza virus or contraction of an influenza virus symptom, thus preventing the disease or reducing the severity of disease symptoms. One or more polynucleotides, polypeptides, vectors, e.g., MVAs, or compositions of the invention can also be used to treat an animal already exposed to an influenza virus, or already suffering from an influenza virus-related symptom to farther stimulate the immune system of the animal, thus reducing or eliminating the symptoms associated with that exposure. As defined herein, “treatment of an animal” refers to the use of one or more polynucleotides, polypeptides, vectors, e.g., MVAs, or compositions comprising the polynucleotides, polypeptides, vectors, e.g., MVAs, to prevent, cure, retard, or reduce the severity of the symptoms caused by an influenza virus infection, e.g., flu, in an animal, and/or result in no worsening of the symptoms over a specified period of time. It is not required that any polynucleotides, polypeptides, vectors, e.g., MVAs, or compositions of the present invention provides total protection against an influenza virus infection or totally cure or eliminate all symptoms related to an influenza virus infection. As used herein, “an animal in need of therapeutic and/or preventative immunity” refers to an animal which it is desirable to treat, i.e., to prevent, cure, retard, or reduce the severity of symptoms related to an influenza virus infection, and/or result in no worsening of the symptoms over a specified period of time.

Treatment with pharmaceutical compositions comprising the polynucleotide, polypeptide, or a vector, e.g., MVA, can occur separately or in conjunction with other treatments, as appropriate.

In therapeutic applications, polynucleotides, polypeptides, vectors, e.g., MVAs, or compositions of the invention are administered to a patient in an amount sufficient to elicit an effective CTL response to the influenza virus-derived polypeptide to cure or at least partially arrest symptoms and/or complications. An amount adequate to accomplish this is defined as “therapeutically effective dose” or “unit dose.” Amounts effective for this use will depend on, e.g., the polynucleotides, polypeptides, vectors, e.g., MVAs, or compositions of the instant invention, the manner of administration, the stage and severity of the disease being treated, the weight and general state of health of the patient, and the judgment of the prescribing physician. In general, ranges for the initial immunization for MVA vaccines is (that is for therapeutic or prophylactic administration) from about 100 pfu to about 1×10¹⁵ pfu of MVA, in some embodiments about 10⁵ pfu to about 10⁹ pfu of MVA, followed by boosting dosages of from about 10³ pfu to about 10⁸ pfu, in some embodiments 10⁶ pfu to about 10⁹ pfu of MVA pursuant to a boosting regimen over weeks to month, depending upon the patient's response and condition by measuring specific CTL activity in the patient's blood. In alternative embodiments, generally for humans the dose range for the initial immunization (that is for therapeutic or prophylactic administration) is from about 10 pfu to about 1×10²⁰ pfu of MVA, for a 70 kg patient, in some embodiments 1000 pfu, 5×10⁴ pfu, 10⁵ pfu, 5×10⁵ pfu, 10⁶ pfu, 5×10⁶ pfu, 10⁷ pfu, 5×10⁷ pfu, 10⁸ pfu, 5×10⁸ pfu, 10⁹ pfu, or 10¹⁰ pfu, followed by boosting dosages in the same dose range pursuant to a boosting regimen over weeks to months depending upon the patient's response and condition by measuring specific CTL (cytotoxic T lymphocytes) activity in the patient's blood. In a specific, non-limiting embodiment of the invention, approximately 10 pfu to about 1×10¹⁵ pfu, or in some embodiments 10⁴ pfu to about 1×10¹⁰ pfu or 10⁷ pfu to 10⁹ pfu, of a MVA of the present invention, or its fragment, derivative variant, or analog is administered to a host.

In non-limiting embodiments of the invention, an effective amount of a composition of the invention produces an elevation of antibody titer to at least two or three times the antibody titer prior to administration.

It must be kept in mind that the polynucleotides, polypeptides, vectors, e.g., MVAs, and compositions of the present invention may generally be employed in serious disease states, that is, life-threatening or potentially life threatening situations. In such cases, in view of the minimization of extraneous substances and the relative nontoxic nature of the polypeptides, it is possible and may be felt desirable by the treating physician to administer substantial excesses of these polypeptide compositions.

For therapeutic use, administration should begin at the first sign of influenza virus infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

More specifically, the compositions of the present invention may be administered to any tissue of an animal, including, but not limited to, muscle, skin, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, or connective tissue, e.g., cartilage.

Furthermore, the compositions of the present invention may be administered to any internal cavity of a vertebrate, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland, or a liver. When the compositions of the present invention are administered to the lumen of a duct of a salivary gland or a liver, the desired polypeptide is encoded in each of the salivary gland and the liver such that the polypeptide is delivered into the blood stream of the vertebrate from each of the salivary gland and the liver. Certain modes for administration to secretory organs of a gastrointestinal system using the salivary gland, liver and pancreas to release a desired polypeptide into the bloodstream is disclosed in U.S. Pat. Nos. 5,837,693 and 6,004,944, both of which are incorporated herein by reference in their entireties.

In certain embodiments, one or more compositions of the present invention are delivered to an animal by methods described herein, thereby achieving an effective immune response, and/or an effective therapeutic or preventative immune response. Any mode of administration can be used so long as the mode results in the delivery and/or expression of the desired polypeptide in the desired tissue, in an amount sufficient to generate an immune response to an influenza virus and/or to generate a prophylactically or therapeutically effective immune response to an influenza virus, in an animal in need of such response. According to the disclosed methods, compositions of the present invention can be administered by mucosal delivery, transdermal delivery, subcutaneous injection, intravenous injection, oral administration, pulmonary administration, intramuscular (i.m.) administration, or via intradural injection. Other suitable routes of administration include, but not limited to intratracheal, transdermal, intraocular, intranasal, inhalation, intracavity, intraductal (e.g., into the pancreas) and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), intraatrial (i.e., into the heart atrium) and sub arachnoid (i.e., into the sub arachnoid spaces of the brain) administration.

Any mode of administration can be used so long as the mode results in the delivery and/or expression of the desired polypeptide in the desired tissue, in an amount sufficient to generate an immune response to an influenza virus, and/or to generate a prophylactically or therapeutically effective immune response to an influenza virus in an animal in need of such response. Administration means of the present invention include needle injection, catheter infusion, biolistic injectors, particle accelerators (e.g., “gene guns” or pneumatic “needleless” injectors) Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet (Linmayer, I., et al., Diabetes Care 9:294-297 (1986)), Medi-jector (Martins, J., and Roedl, E. J. Occup. Med. 21:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65, 2193-2203 (1999)) or topical applications during surgery. Certain modes of administration are intramuscular needle-based injection and pulmonary application via catheter infusion. Each of the references cited in this paragraph is incorporated herein by reference in its entirety.

Upon immunization with the polynucleotides, polypeptides, vectors, e.g., MVAs, or compositions in accordance with the invention, the immune system of the host responds to the vaccine by producing large amounts of HTLs (helper T lymphocytes) and/or CTLs (cytotoxic T lymphocytes) specific for the desired antigen. Consequently, the host becomes at least partially immune to later infection, or at least partially resistant to developing an ongoing chronic infection.

In some embodiments, polynucleotides, polypeptides, vectors, e.g., MVAs, or compositions of the present invention stimulate a cell-mediated immune response sufficient for protection of an animal against an influenza viral infection. In other embodiments, polynucleotides, polypeptides, vectors, e.g., MVA, or compositions o the invention induce a humoral immune response. In certain embodiments, polynucleotides, polypeptides, or vectors, e.g., MVA, of the present invention stimulate both a humoral and a cell-mediated response, the combination of which is sufficient for protection of an animal against influenza virus infection.

In still other embodiments, components that induce T cell responses are combined with components that induce antibody responses to the target antigen of interest. Thus, in certain embodiments of the invention, vaccine compositions of the invention are combined with polypeptides or polynucleotides which induce or facilitate neutralizing antibody responses to the target antigen of interest. One embodiment of such a composition comprises a class I epitope in accordance with the invention, along with a PADRE® (Epimmune, San Diego, Calif.) molecule (described, for example, in U.S. Pat. No. 5,736,142, which is incorporated herein by reference in its entirety.).

Polynucleotides, polypeptides, vectors, e.g., MVAs, or compositions comprising the polynucleotides, polypeptides, or vectors, e.g., MVAs, can be incorporated into the cells of the animal in vivo, and an antigenic amount of the influenza M2-derived polypeptide, or fragments, variants, or derivatives thereof, is produced in vivo. Upon administration of the composition according to this method, the METR polypeptide is expressed in the animal in an amount sufficient to elicit an immune response. Such an immune response might be used, for example, to generate antibodies to an influenza virus for use in diagnostic assays or as laboratory reagents.

The present invention further provides a method for generating, enhancing, or modulating a protective and/or therapeutic immune response to an influenza virus in an animal, comprising administering to the animal in need of therapeutic and/or preventative immunity one or more of the compositions described herein. In some embodiments, the composition includes a recombinant MVA containing a polynucleotide comprising a codon-optimized coding region encoding a polypeptide of the present invention, optimized for expression in a given host organism, e.g., a human, or a nucleic acid fragment of such a coding region encoding a fragment, variant, or derivative thereof. The recombinant MVA is incorporated into the cells of the animal in vivo, and an immunologically effective amount of the influenza viral polypeptide, or fragment or variant is produced in vivo. Upon administration of the composition according to this method, the influenza virus-derived polypeptide is expressed in the animal in a therapeutically or prophylactically effective amount.

The compositions of the present invention can be administered to an animal at any time during the lifecycle of the animal to which it is being administered. For example, the composition can be given shortly after birth. In humans, administration of the composition of the present invention can occur while other vaccines are being administered, e.g., at birth, 2 months, 4 months, 6 months, 9 months, at 1 year, at 5 years, or at the onset of puberty. In some embodiments, administration of the composition of the present invention can occur before initiation of an immune-suppressing treatment.

Furthermore, the compositions of the invention can be used in any desired immunization or administration regimen; e.g., in a single administration or alternatively as part of periodic vaccinations such as annual vaccinations, or as in a prime-boost regime wherein the polypeptide or polynucleotide of the present invention is administered either before or after the administration of the same or of a different polypeptide or polynucleotide.

Recent studies have indicated that a prime-boost protocol is often a suitable method of administering vaccines. In a prime-boost protocol, one or more compositions of the present invention can be utilized in a “prime boost” regimen. An example of a “prime boost” regimen may be found in Yang, Z. et al. J. Virol. 77:799-803 (2002), which is incorporated herein by reference in its entirety. In a non-limiting example, one or more vaccine compositions comprising the polynucleotides, polypeptides, or vectors, e.g., MVAs, of the present invention are delivered to an animal, thereby priming the immune response of the animal to an influenza M2 polypeptide, and then a second immunogenic composition is utilized as a boost vaccination.

In another non-limiting example, a priming composition and a boosting composition are combined in a single composition or single formulation. For example, a single composition may comprise an isolated polynucleotide or vector, e.g., comprising a polynucleotide encoding an influenza protein, fragment, variant, derivative, or analogue thereof or an isolated polypeptide comprising an influenza protein, fragment, variant, derivative, or analogue thereof as the priming component and a polynucleotide, polypeptide, or vector, e.g., MVA, of the present invention as the boosting component. In this embodiment, the compositions may be contained in a single vial where the priming component and boosting component are mixed together. In general, because the peak levels of expression of polypeptide from the polynucleotide does not occur until later (e.g., 7-10 days) after administration, the polynucleotide component may provide a boost to the isolated polypeptide component. Compositions comprising both a priming component and a boosting component are referred to herein as “combinatorial vaccine compositions” or “single formulation heterologous prime-boost vaccine compositions.” In addition, the priming composition may be administered before the boosting composition, or even after the boosting composition, if the boosting composition is expected to take longer to act.

In another embodiment, the priming composition may be administered simultaneously with the boosting composition, but in separate formulations where the priming component and the boosting component are separated.

### Kits

The polynucleotide, polypeptide, or vector, e.g., MVA, or compositions of this invention can be provided in kit form together with a means for administering the recombinant MVA or composition of the present invention. In some embodiments, the kit can further comprise instructions for vaccine administration.

Typically the kit would include desired composition(s) of the invention in a container, e.g., in unit dosage form and instructions for administration. Means for administering the composition of the present invention can include, for example, a sterile syringe, an aerosol applicator (e.g., an inhaler or any other means of nasal or pulmonary administration), a gel, a cream, a transdermal patch, transmucosal patch (or any other means of buccal or sublingual administration), or an oral tablet. In some embodiments, the kit of the present invention contains two or more means for administering the polypeptides, polynucleotides, vectors, or compositions of the present inventions, e.g., two or more syringes.

In some embodiments, the kit may comprise more than one container comprising the polypeptide, polynucleotide, or composition of the present invention. For example. in some embodiments the kit may comprise a container containing a priming component of the present invention, and a separate container comprising the boosting component of the present invention.

Optionally associated with such container(s) can be a notice or printed instructions. For example, such printed instructions can be in a form prescribed by a governmental agency regulating the manufacture, use or sale of pharmaceuticals or biological products, which notice reflects approval by the agency of the manufacture, use or sale for human administration of the present invention. “Printed instructions” can be, for example, one of a book, booklet, brochure or leaflet.

The kit can also include a storage unit for storing the components (e.g., means of administering, containers comprising the recombinant MVA or compositions of the present inventions, printed instructions, etc.) of the kit. The storage unit can be, for example, a bag, box, envelope or any other container that would be suitable for use in the present invention. For example, the storage unit is large enough to accommodate each component that may be necessary for administering the methods of the present invention.

The present invention can also include a method of delivering a recombinant MVA or composition of the present invention to an animal such as a human in need thereof, the method comprising (a) registering in a computer readable medium the identity of an administrator (e.g., a physician, physician assistant, nurse practitioner, pharmacist, veterinarian) permitted to administer the polypeptide, polynucleotide, vector, or composition of the present invention; (b) providing the human with counseling information concerning the risks attendant the polypeptide, polynucleotide, vector, or composition of the present invention; (c) obtaining informed consent from the human to receive the polypeptide, polynucleotide, vector, or composition of the present invention despite the attendant risks; and (e) permitting the human access to the polypeptide, polynucleotide, vector, or composition of the present invention.

### EXAMPLES

### Example 1

### Construction of Recombination Vector

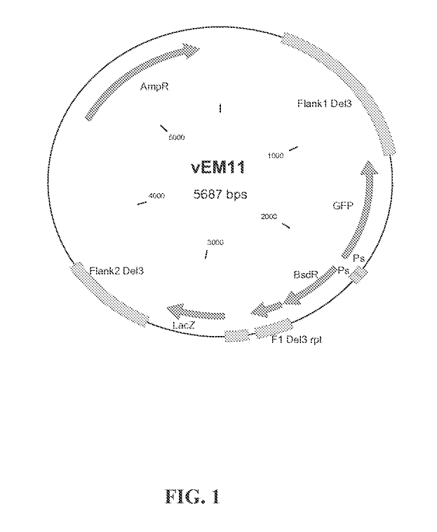

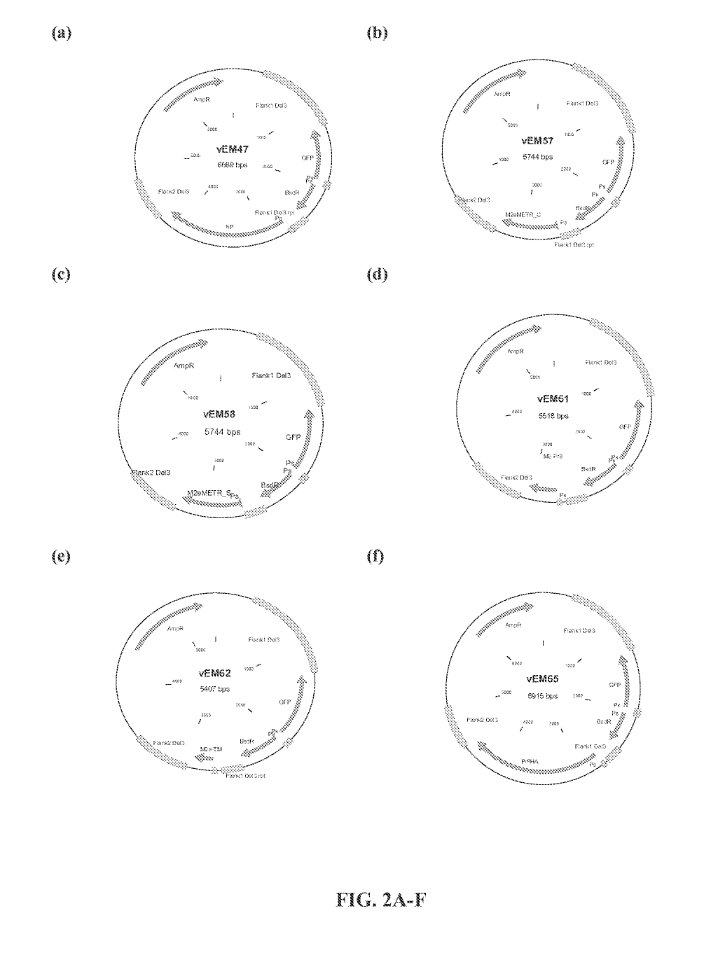

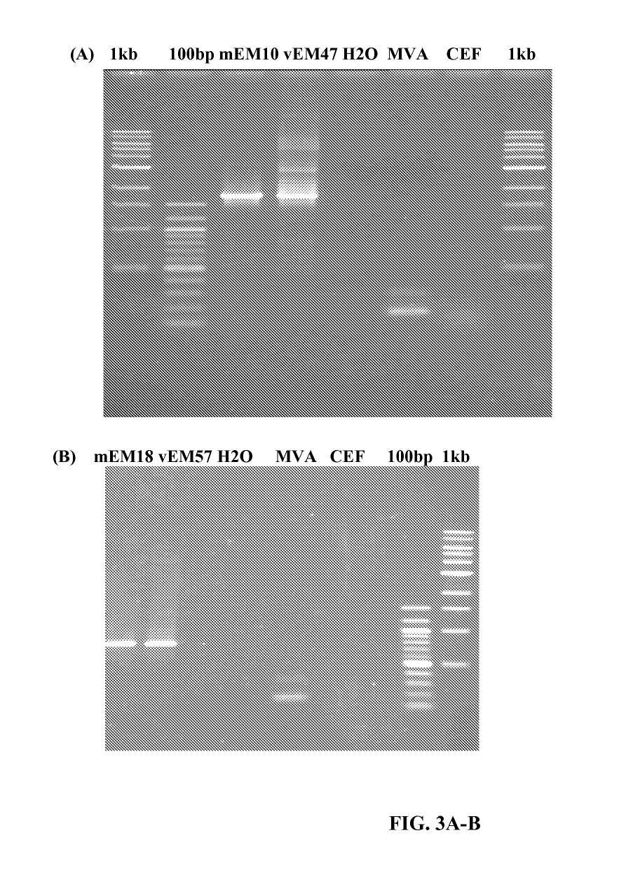

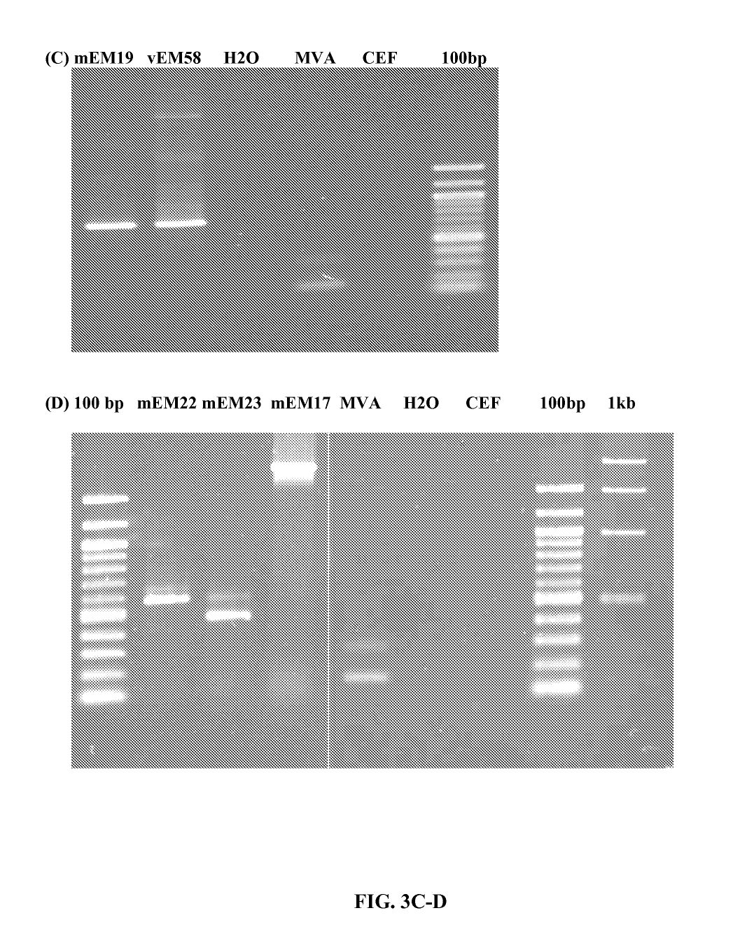

The sequences of Influenza A genes Pr8HA (SEQ ID NOs: 51-52), NP consensus (SEQ ID NOs: 19-20), Pr8M2 (SEQ ID NOs: 13-14), Pr8M2e_TML (SEQ ID NOs: 53-54), METR_S (SEQ ID NOs: 17-18 and 56), and METR_C (SEQ ID NOs: 15-16 and 55) were cloned in the vEM11 recombination vector (FIG. 1). The resulting recombination vectors vEM47 (coding for the NP consensus sequence), vEM58 (coding for the METR_S peptide), vEM57 (coding for the METR_C peptide), vEM61 (coding for Pr8M2), vEM62 (coding for Pr8M2e-TML) and vEM65 (coding for Pr8HA) are shown in FIG. 2A-F.

### Example 2

### Homologous Recombination and Isolation of Recombinant Virus

For the insertion of the influenza genes in a modified MVA viral vector, MVAtor™ (Emergent Biosolutions), CEF cells were infected with MVAtor and subsequently transfected with the recombination vectors shown in FIG. 2A-F. Two to three set-ups in parallel per each vector were performed. First, 5×10⁵ CEF cells were seeded per well of a six well plate and incubated for 24 hours at 37° C. and 5% CO₂. On the next day, the cell density was assumed to be 10⁶ cells per well. The MVAtor standard was diluted in Opti-Pro SFM containing 4 mM L-Glutamine and 2.5 μg Gentamicin per ml so that 500 μl did contain 5×10⁴ TCID₅₀, i.e., a working concentration of 1×10⁵ TCID₅₀/ml and resulted in a moi of 0.05. For the infection, the growing medium was removed from cells, 500 μl diluted MVAtor standard were added per well and incubated for one hour at room temperature while rocking. The virus inoculum was removed, and the cells were washed with Opti-Pro SFM. The infected cells were left in 2.0 ml of Opti-Pro SFM while transfection reaction was set up.

The transfection reactions were set up in a sterile 5 ml PS tube. Approximately 1.0 to 2.0 μg DNA of the recombination vector and 6 μl Transfection reagent (FuGene HD) were used for transfection that was performed according to the FuGENE HD standard protocol provided by the supplier. The cells were incubated for 48 hours at 37° C. and 5% CO₂ in an incubator.

After the incubation, the cells were screened on fluorescing cells that indicate the presence of MVAtor and recombination vector within the cells. Cells were screened on fluorescing foci and set ups with the most efficient gfp expression were used for two to three passages of the recombinant MVAtor under selective conditions. For this passage, the infected transfected cells were scraped into the medium by using a cell lifter and transferred to a 1.5 ml vial. Viruses were released by ultrasound treatment according to EPDD-SOP-EQU-033. 1/10-½ of the transfection set up was plated on fresh CEF cells seeded in a 12-well plate, filled ad 1 ml and 5 μg Blasticidine per ml were added.

In order to plaque purify the recombinant MVAtors, recombinant MVAtor was seeded in serial dilutions in 6-well plates and 96-well plates, respectively, under selective conditions (5 μg/ml Blasticidine per ml Opti-Pro SFM). Single fluorescing plaques were isolated by using a 100 μl pipette. The isolated virus was transferred to a 1.5 ml vial and released by ultrasound treatment. The virus was analyzed by PCR on empty vector (as described below) and the isolate showing the weakest empty vector signal is passaged on fresh CEF cells seeded in 12-well plates. The plaque purification was repeated until the isolate was free of empty vectors. As soon as a pure clone was isolated, the virus was passaged without blasticidine and the non fluorescing viruses devoid of selection/reporter cassette were isolated.

For the deletion of the selection cassette, the pure recombinant viruses were passaged without Blasticidine. Plaques without fluorescence were isolated and tested on the insertion of the expected Influenza A gene in the genome. The recombinant MVAtors were then amplified up to 3×T175 and a detailed preliminary testing was performed (PCR, Sequencing, expression, titre) in the following virus stocks: (1) MVAtor-NP consensus (mEM10), P21pp8, (2) MVAtor-METR_C (mEM18), P14pp8, (3) MVAtor-METR_S (mEM19), P18pp13, (4) MVAtor-Pr8M2 (mEM22), P13pp3, (5) MVAtor-Pr8M2e_TML (mEM23), P14pp3, and (6) MVAtor-Pr8HA (mEM17). The virus stocks were shown to have a 100% correct sequence read-out and to be free of residual empty vector and functional.

After the isolation of a pure recombinant MVAtor devoid of the selection/reporter cassette, the viruses were amplified on T225 flasks. Ten T225 flask primary CEF cells were split 1:4 in a total amount of 40 T225 flasks for amplification of each individual recombinant MVAtor. After two days of incubation, the cells of one flask were trypsinized and counted. The 39 T225 flasks were infected with a moi of 0.1 and incubated for 72 hours at 37° C. and 5% CO₂. The flasks were harvested and the content of all flasks was pooled. The homogenization pooled virus stock was performed by ultrasound treatment using a flow cell device. The flask containing the virus stock that had to be purified was placed on ice and connected to the flow cell. The ultrasound flow cell device was set as following: Amplitude to 100%, Cycle 1, pump speed of 50 ml/min i.e. 0.8 and switched on. After sounding the stock was frozen until purification.

Considering the titre, the purified stock was diluted and filled to a final filling volume of 600 μl per vial containing 109 TCID₅₀ per ml (nominal titer). The filling resulted in the following amount of vials per virus: (1) 91×MVAtor-NP consensus (mEM10); (2) 35×MVAtor-METR_C (mEM18), (3) 29×MVAtor-METR_S (mEM19), (4) 56×MVAtor-Pr8M2 (mEM22), (5) 38×MVAtor-Pr8M2e_TML (mEM23), and (6) 30×MVAtor-Pr8HA (mEM17). All vials were stored at −70° C. until further use, i.e. testing or shipping. One vial per virus was archived in the −70° C. sample archive.

### Example 3

### PCR for the Exclusion of Empty Vector Contamination

In order to confirm that the residual non-recombinant MVAtors were successfully excluded from the filled vials of the purified virus stocks, DNA from purified and filled MVAtor-NP for mEM10, MVAtor-METR_C for mEM18, MVAtor-METR_S for mEM19, MVAtor-Pr8M2 for mEM22, or MVAtor-Pr8M2e_TML for mEM23 was isolated and used for PCR. As a positive control for recombinant virus, the recombination vector vEM47 was used (vEM47). As positive control for empty vector, DNA isolated from MVAtor was used (MVA). As negative controls, H₂O and CEF cells were used.

### Example 4

### Sequencing for Influenza Genes and Flanking Sequences of Insertion Site

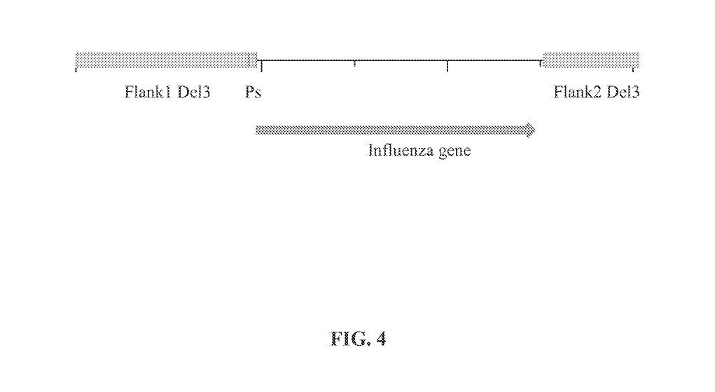

DNA of each virus was isolated and the entire insertion site was amplified by PCR using the primers 5′-ggagctccactatttagttggtggtcgcc-3′ (SEQ ID NO: 32) (oVIV47) and 5′-cgggtaccetagtttccggtgaatgtg-3′ (SEQ ID NO: 33) (oVIV89). Using these primers, the inserted Influenza genes as well as the flanking sequences used for the homologous recombination are amplified (FIG. 4).

The PCR fragments were purified and shipped to GATC for sequencing. For the sequencing, the primers were chosen to cover the entire PCR fragment as provided below:

TABLE 5
Primers
5A. Primers for Sequencing MVAtor-NP consensus and flanking regions (mEM10)

| | | |
|---|---|---|
| oVIV38 oVIV-Del IIIend | ctagatcatcgtatggagagtcg | (SEQ ID NO: 34) |
| oVIV45 oVIV-F2up + ApaI | gaaagttttataggtag | (SEQ ID NO: 35) |
| oVIV48 oVIV-F1end + BstXI | gccaccgcggtggccagccaccgaaagagcaatc | (SEQ ID NO: 36) |
| oVIV49 oVIV-F1mid(rpt) + BglII | ggaagatctcaattaacgatgagtgtag | (SEQ ID NO: 37) |
| oVIV54 oVIV-Del IIIF1-seq | gatgtaggcgaatttggatc | (SEQ ID NO: 38) |
| oVIV53 oVIV-Del IIIF2-seq | tggtaatcgtgtcatattag | (SEQ ID NO: 39) |
| oVIV55 oVIV-Del IIIF1mid rev | cattattatcggttacacttc | (SEQ ID NO: 40) |
| oEM229 NP fw | CAAGAAGTGCTTATGAG | (SEQ ID NO: 41) |
| oEM230 NP rev | ggttccgactttctctcact | (SEQ ID NO: 42) |

5B. Sequencing MVAtor-METR_C and flanking regions (mEM18):

| | | |
|---|---|---|
| oVIV37 oVIV-Del IIIup | ggcacctctcttaagaagtgtaac | (SEQ ID NO: 43) |
| oVIV54 oVIV-Del IIIF1-seq | gatgtaggcgaatttggatc | (SEQ ID NO: 38) |
| oVIV53 oVIV-Dei IIIF2-seq | tggtaatcgtgtcatattag | (SEQ ID NO: 39) |
| oVIV55 oVIV-Del IIIF1mid rev | cattattatcggttacacttc | (SEQ ID NO: 40) |
| oEM283 M2tandem_forward | CTTACAGAAGTGGAGACAC | (SEQ ID NO: 44) |
| oEM284 M2tandem_backward | GTAAGGAGACTCAGCTTC | (SEQ ID NO: 45) |

5C. Sequencing MVAtor-METR_S and flanking regions (mEM19):

| | | |
|---|---|---|
| oVIV37 oVIV-Del IIIup | ggcacctctcttaagaagtgtaac | (SEQ ID NO: 43) |
| oVIV45 oVIV-F2up + ApaI | gaaagttttataggtag | (SEQ ID NO: 35) |
| oVIV53 oVIV-Del IIIF2-seq | tggtaatcgtgtcatattag | (SEQ ID NO: 39) |
| oVIV55 oVIV-Del IIIF1mid rev | cattattatcggttacacttc | (SEQ ID NO: 40) |
| oEM184 Flank1-seq-down | GGATAGAGAIGTTIGIGAAC | (SEQ ID NO: 46) |
| oEM283 M2tandem_forward | CTTACAGAAGTGGAGACAC | (SEQ ID NO: 47) |
| oEM284 M2tandem_backward | GTAAGGAGACTCAGCTTC | (SEQ ID NO: 48) |

5D. Sequencing MVAtor-Pr8M2 and flanking regions (mEM22):

| | | |
|---|---|---|
| oVIV37 oVIV-Del IIIup | ggcacctctcttaagaagtgtaac | (SEQ ID NO: 43) |
| oVIV45 oVIV-F2up + ApaI | gaaagttttataggtag | (SEQ ID NO: 35) |
| oVIV47 oVIV-F1up + SacI | ggagctccactatttagttggtggtcgcc | (SEQ ID NO: 32) |
| oVIV53 oVIV-Del IIIF2-seq | tggtaatcgtgtcatattag | (SEQ ID NO: 39) |
| oVIV54 oVIV-Del IIIF1-seq | gatgtaggcgaatttggatc | (SEQ ID NO: 38) |
| oVIV55 oVIV-Del IIIF1mid rev | cattattatcggttacacttc | (SEQ ID NO: 40) |
| oVIV89 F2end Acc65Inew | cgggtaccctagtttccggtgaatgtg | (SEQ ID NO: 33) |

5E. Sequencing MVAtor-Pr8M2e_TML and flanking regions (mEM23):

| | | |
|---|---|---|
| oVIV37 oVIV-Del IIIup | ggcacctctcttaagaagtgtaac | (SEQ ID NO: 43) |
| oVIV45 oVIV-F2up + ApaI | gaaagttttataggtag | (SEQ ID NO: 35) |
| oVIV47 oVIV-F1up + SacI | ggagctccactatttagttggtggtcgcc | (SEQ ID NO: 32) |
| oVIV48 oVIV-F1end + BstXI | gccaccgcggtggccagccaccgaaagagcaatc | (SEQ ID NO: 36) |
| oVIV53 oVIV-Del IIIF2-seq | tggtaatcgtgtcatattag | (SEQ ID NO: 39) |
| oVIV54 oVIV-Del IIIF1-seq | gatgtaggcgaatttggatc | (SEQ ID NO: 38) |
| oVIV55 oVIV-Del IIIF1mid rev | cattattatcggttacacttc | (SEQ ID NO: 40) |
| oVIV89 F2end Acc65Inew | cgggtaccctagtttccggtgaatgtg | (SEQ ID NO: 33) |

5F. Sequencing MVAtor-Pr8HA and flanking regions (mEM17):

| | | |
|---|---|---|
| oVIV37 oVIV Del III up | ggtggtgagttgaaggattcacttcc | (SEQ ID NO: 43) |
| oVIV38 oVIV-Del III end | ctagatcatcgtatggagagtcg | (SEQ ID NO: 34) |
| oVIV45 oVIV-F2up + ApaI | gaaagttttataggtag | (SEQ ID NO: 35) |
| oVIV47 oVIV-F1up + SacI | ggagctccactatttagttggtggtcgcc | (SEQ ID NO: 32) |
| oVIV48 oVIV-F1end + BstXI | gccaccgcggtggccagccaccgaaagagcaatc | (SEQ ID NO: 36) |
| oVIV53 oVIV-Del IIIF2-seq | tggtaatcgtgtcatattag | (SEQ ID NO: 39) |
| oVIV55 oVIV-Del IIIF1mid rev | cattattatcggttacacttc | (SEQ ID NO: 40) |
| oEM280 Pr8 HA_mid_backward | GTTACACTCATGCATTGATG | (SEQ ID NO: 49) |
| oEM281 Pr8 HA_mid_forward | CAAATGGAAATCTAATAGCAC | (SEQ ID NO: 50) |

### Example 5

### Expression Analysis of Influenza Polypeptides

Western blot was performed to analyze expression of MVAtor-NP as well as both-METR constructs (SEQ ID NO: 16 for the METR_C polypeptide and SEQ ID NO: 18 for the METR_S polypeptide). In a six well plate, 6×10⁵ cells were seeded per well in the appropriate amount of wells. The amount of wells was determined as follows: number of recombinant MVAtor samples plus MVAtor control plus CEF control. Cells were infected with MVAtor and MVAtor-NP/-METR_S, -METR_C, respectively, using a moi of 1 according to standard protocols. After 24 hours of infection, 300 μl RIPA buffer (pre cooled on ice) per well were added, and the plates were incubated for 5 min on ice. The cells were scraped into the RIPA buffer and the cell suspensions were transferred each in a 1.5 ml vial and placed on ice. Protease inhibitor cocktail in an amount of 0.5 μl was added to each vial. A volume of 60 μl per sample was transferred into a new 1.5 ml vial and 22 μl loading dye, and 8 μl 2-Mercaptoethanol was added. For the Influenza virus positive control, 5 μl of inactivated Influenza virus was used. A volume of 8 μl RIPA buffer was added. All samples were incubated on ice for 5 min. Afterwards all samples were heated for 10 min. at 95° C. in a thermo mixer.

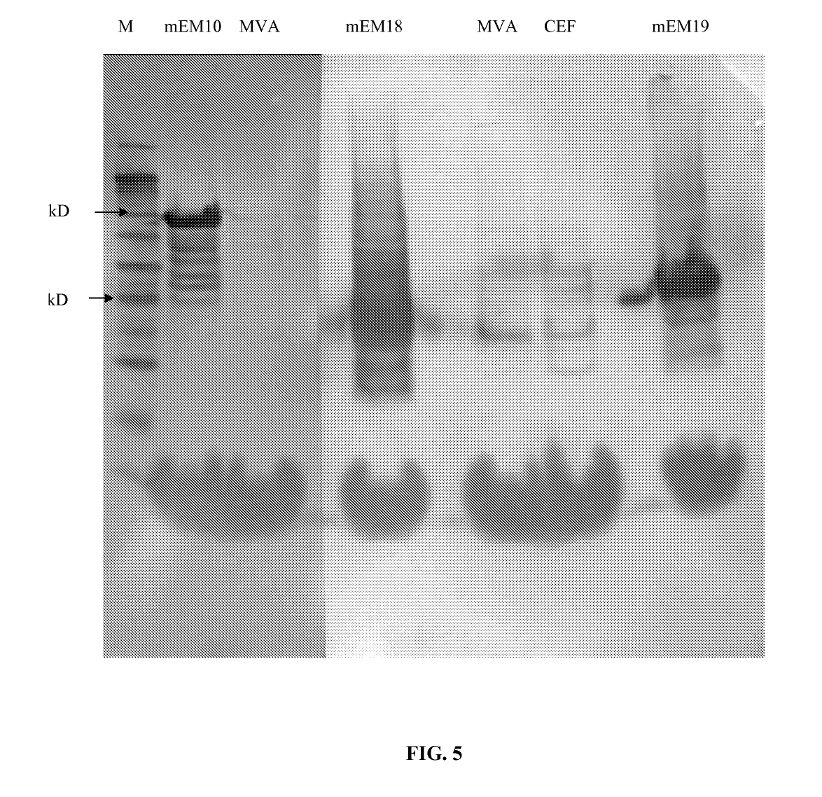

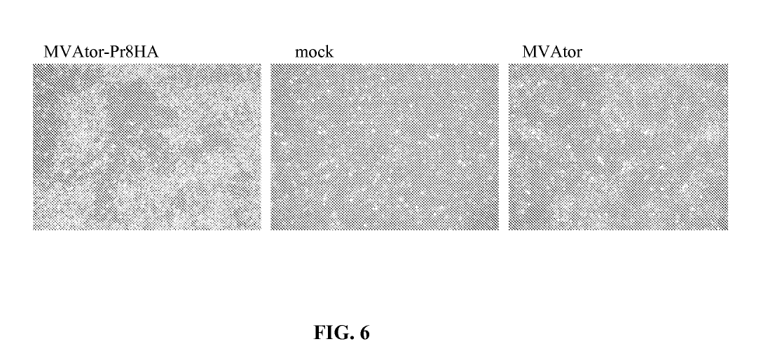

A haemadsorption assay (HAD) was performed to analyze expression of the MVAtor-Pr8HA. For this purpose CEF cells were infected with MVAtor and MVAtor-HA (mEM17), respectively. Another set of cells was mock infected. Twenty four hours after the infection, the cells were incubated with 1% erythrocyte dilution of human blood (FIG. 6).

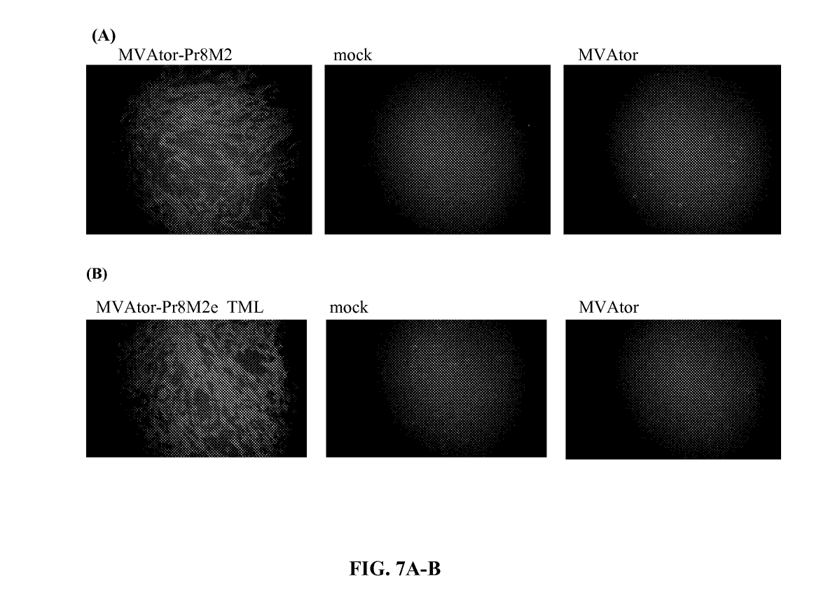

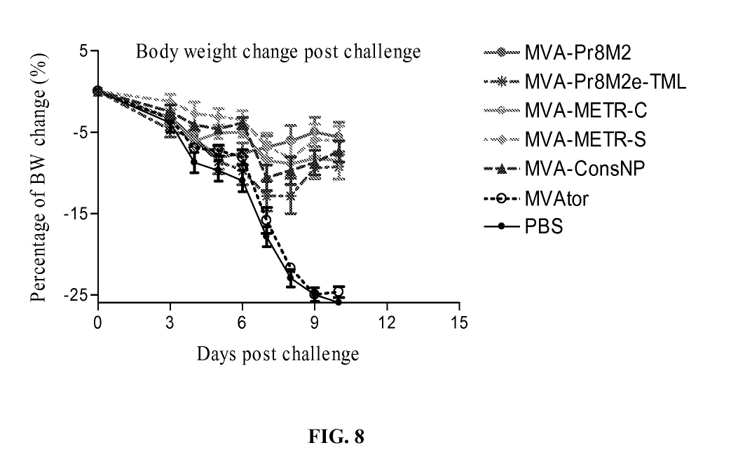

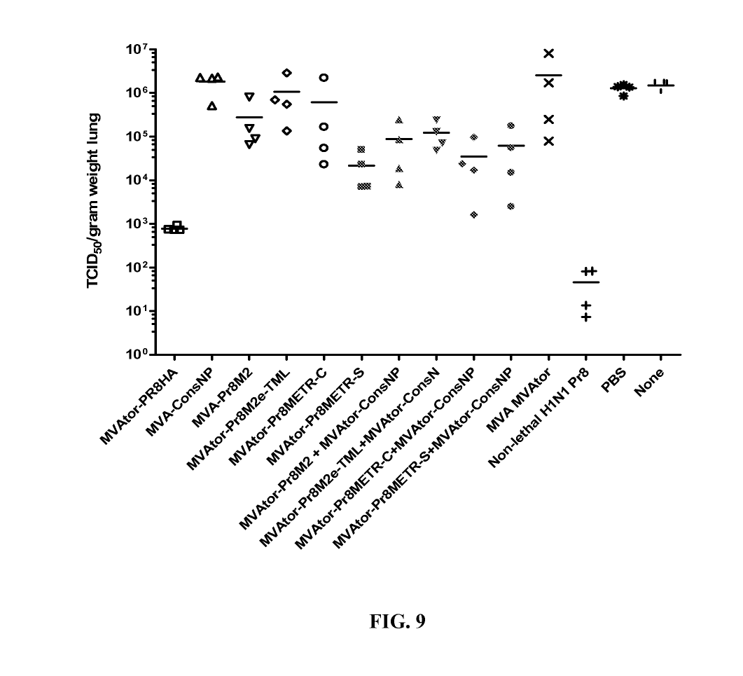

An immunoassaying was performed to analyze expression of MVAtor-M2 and MVAtor-M2e-TML (FIG. 7). Cells were infected with MVAtor-Pr8M2 (FIG. 7A) and MVAtor-Pr8M2e-TML (FIG. 7B). In parallel, cells were infected with MVAtor or were incubated without infection.

### Example 6

### In Vivo Efficacy of the MVA Vaccines—Body Weight and Viral Burden

The MVAtors expressing either METR-C (SEQ ID NO: 16) or METR-S (SEQ ID NO: 18) were tested for their in vivo efficacy. Efficacy of the MVA vaccines was observed by assessing 1) weight loss or mortality of the mice in the challenged groups compared with the control groups, and 2) viral burden in the lungs. Groups of mice (eight mice per group) were immunized twice intramuscularly with the MVA vaccines: (1) MVA-Pr8M2 (MVA construct expressing full-length M2 of influenza A virus Puerto Rico 1934 H1N1 (Pr8)); (2) MVA-Pr8M2e-TML (MVA construct expressing the native transmembrane region of M2 (TML)); (3) MVA-METR-C (MVA construct expressing the METR polypeptide having cysteins); (4) MVA-METR-S (MVA construct expressing the METR polypeptide in which cysteines had been substituted with Serines); (5) MVA-ConsNP (MVA construct expressing NP consensus sequence); (6) MVAtor (MVA vector alone); and (7) PBS as a negative control.

Three weeks after the immunization, mice were infected intrapulmonarily with 50 mcL of Influenza A virus (A/PR/8/34, H1N1) at 629 TCID50 per mouse. Mice were monitored daily for body weight reduction. The data are the mean±SEM (percentage) compared to body weight before challenge. Mice were euthanized if BW reduction reached 25%. As a result, the negative control mice suffered weight loss and died. Weight loss was measurable in mice that received an MVA-M2eTML construct (14%) but the METR constructs resulted in only 7% weight loss, similar to full length M2 (Pr8M2).

The quantitative recovery of influenza viruses from the infected mice lungs was performed, and viral burdens were compared in order to determine immune benefits due to the previous vaccinations.

Groups of four cryotubes containing individual mouse lungs were removed from −80° C. and placed on ice to thaw. Each lung was weighed. L-15-2×PSK media (Leibovitz+4 mM L-Glutamine+2× antibiotic-antimycotic) was transferred to each tube so that the lung weight is equal to 10% of the total volume. Each lung was homogenized using Power Gen 125 and disposable homogenizers until complete (30 seconds to 1 minute). Lung homogenates were spun down 20 minutes at 30000 rpm at 4° C. Lung homogenate supernatants were aliquoted; 200 mcL for TCID50 and two aliquots of 400 mcL freeze directly in dry ice. The aliquots were stored at −80° C.

Lung homogenates were vortexed, and 20 mcL was added to 4 wells containing MDCK cells plated 18-24 hours previously at 4×10e5 cells in 180 mcL of serum free MEM containing Trypsin (TPCK) and 4 mM Glutamine and 2× antibiotic-antimycotic. Serial 10 fold dilutions were performed down the plate by transferring 20 mcL from row A to 180 mcL of media in row B and continuing through row H. Influenza Virus A/PR/8/34 stock ATCC VR-9 was used to infect MDCK cells in 4 wells of every third assay plate as a positive control. Source of positive control A/PR/8/34 was from three vial sources stored at −80° C. freezer that had temperature failure. Infected MDCK cells were incubated at 37° C./5% CO₂ for 4 days. Plates were washed once with PBS, and MDCK cells were incubated at 4° C. for 30 minutes with 0.1% Turkey red bloods cells. Unattached Turkey red blood cells were washed vigorously with PBS four times and infected MDCK cells were visible by adherent red blood cells. Log 10 TCID50 titer was calculated from counting infected wells of MDCK cells. Results expressed TCID50/gram weight lung tissue by calculation of the anti-log of Log 10TCID50 divided by gram weight of lung tissue for each mouse. Mean, standard deviation and Coefficient of variation expressed as % were determined for each group.

### Example 7

### Antibody Response Against M2e in Immunized Mice

In order to test antibody titer of the immunized animal, mice were immunized intramuscularly once, twice, or thrice with the MVA vaccines: (1) MVA-Pr8M2, (2) MVA-Pr8M2e-TML; (3) MVA-METR-C, (4) MVA-METR-S, (5) MVA-Pr8M2_MVA-ConsNP; (6) MVA-Pr8M2e-TML+MVA-ConsNP; (7) MVA-Pr8M2e-TML+MVA-ConsNP; (8) MVA-ConsNP, (9) Control group, (10) MVA virus with insert (no influenza antigen expressed); (11) sublethal infection using PR8 infective influenza virus (No MVA vaccine); and (12) PBS as a positive control. After the final immunization, the mice were challenged using infective influenza virus (A/Pr/8/34). To perform the M2 ELISA, biotinylated peptides consisting of the M2e regions were immobilized on to the ELISA plates via a form of avidin. Of the 6 peptides represented within an M2 Ectodomain Tandem Repeat (METR), representative anti-M2e titers were obtained by using ELISA plate coats of Peptide 4, representing influenza A Puerto Rico 1934 H1N1. Serum titers for peptide #4 (SEQ ID NO: 4) recognition are summarized in Table 6.

TABLE 6
Anti-M2e peptide #4 Antibody Response (μg/mL, Geomean ±
geometric SD)

| | | | |
|---|---|---|---|
| Test antigen inserted | | | |
| into MVA vectored | | | |
| influenza vaccine, and | | | |
| delivered to mice at a | | | |
| dose level of 8 × 10e7 | | | |
| MVA TCID50 | Ab after one | Ab after two | Ab after three |
| per mouse. | vaccination | vaccinations | vaccinations |

Test Groups

| | | | |
|---|---|---|---|
| Pr8M2 | 0.2 ± (−1.6) | 7.2 ± 2.0* | 6.4 ± 1.9* |
| Pr8M2e-TML | 0.3 ± (−1.3) | 6.5 ± 1.9* | 16.1 ± 2.8* |
| METR-C | 0.6 ± (−0.5) | 46.3 ± 3.8*@ | 103 ± 4.6*#@ |
| METR-S | 6.3 ± 1.8 | 73.5 ± 4.3*@ | 43.4 ± 3.8*@ |
| Pr8M2 + ConsNP | 0.5 ± (−0.6) | 10.6 ± 2.4* | NA |
| Pr8M2e-TML + ConsNP | 0.3 ± (−1.1) | 12.9 ± 2.6* | NA |
| METR-C + ConsNP | 1.0 ± 0.0 | 54.3 ± 4.0*@ | NA |
| METR-S + ConsNP | 0.3 ± (−1.1) | 12.9 ± 2.6* | NA |
| ConsNP | 0.1 | 0.1 | ND |

Control Groups

| | | | |
|---|---|---|---|
| MVA virus with insert | 0.1 | 0.1 | 0.1 |
| (no influenza antigen | | | |
| expressed) | | | |
| Sublethal infection using | 2.6 | 3.4 ± 0.4 | 0.6 ± 0.4 |
| PR8 infective | | | |
| influenza virus | | | |
| (No MVA vaccine) | | | |
| PBS (No MVA vaccine) | 0.1 | 0.1 | NA |

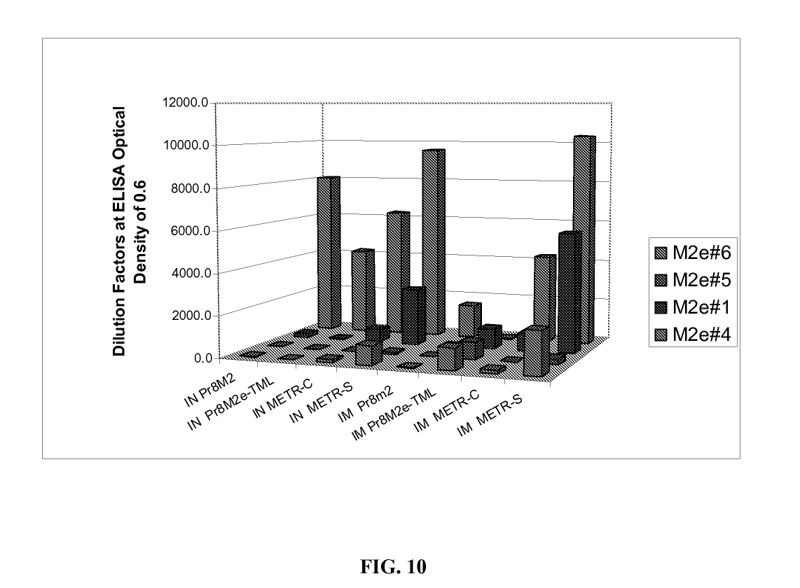

As shown in FIG. 10, the MVA-METR vaccines expressing multiple M2e regions generated higher titered serum levels of IgG anti-M2 peptide than the MVA-M2 vaccines did.

In a further study, BALB/c mice were immunized twice either intranasally to the lung (IN) or intramuscularly (IM) with the MVA vaccines: (1) sublethal dose of influenza A virus Puerto Rico 1934 H1N1 (positive control), (2) MVA-HA (MVA construct expressing full-length HA); (3) MVA-METR-S (MVA construct expressing METR with Serine substitutions); (4) MVA-M2 (MVA construct expressing full-length M2); (5) MVA-METR-C (MVA construct expressing METR with the native cysteines); (6) MVA-Pr8M2e-TML (MVA construct that contained the native transmembrane region of M2 (TML); (7) PBS (negative control); (8) MVA-ConsNP (MVA construct expressing NP consensus); and (9) MVAtor-alone (negative control). Immune sera were obtained 21 days after the second immunization of the vaccines and were tested by ELISA, which was coated with one of the four peptides, each representing different strains of influenza A virus protein: M2e#1 H5 1999 to 2008 (bars second from back of graph); M2e#4 H1 and H3 human (bars at back of graph); M2e#5 H9 and H6 (bars second from front of graph); M2e#6 H7 and H3, H8, H10, H2, H6, H9 (bars at front of graph).

TABLE 7
Anti-M2e peptide#4 antibody responses(μg/mL, Geomean ±
geometric SD)

| Antigen, | | | | |
|---|---|---|---|---|
| expressed by | Intranasal delivery | | Intramuscular injection | |
| MVA vector | Ab after one | Ab after two | Ab after one | Ab after two |
| per vaccine | vaccination | vaccinations | vaccination | vaccinations |
| Pr8M2 | 0.4 ± (−1.0) | 32.6 ± 3.5* | 0.4 ± (−1.0) | 8.8 ± 2.2* |
| Pr8M2e-TML | 0.2 ± (−1.7) | 26.7 ± 3.3* | 0.2 ± (−1.5) | 6.5 ± 1.9* |
| METR-C | 0.2 ± (−15) | 29.1 ± 3.4* | 0.5 ± (−0.7) | 20.7 ± 3.0* |
| METR-S | 1.3 ± 0.3 | 39.1 ± 3.7* | 4.6 ± 1.5 | 62.5 ± 4.1* |
| No influenza | 0.1 | 0.1 | 0.1 | 0.1 |
| antigen. | | | | |
| MVA alone | | | | |
| No influenza | 0.1 | 0.1 | NA | NA |
| antigen. | | | | |
| PBS alone | | | | |

Body weight changes in the intranasal (IN) or intramuscular (IM) immunized mice immunized are shown in FIG. 11A and FIG. 11B.

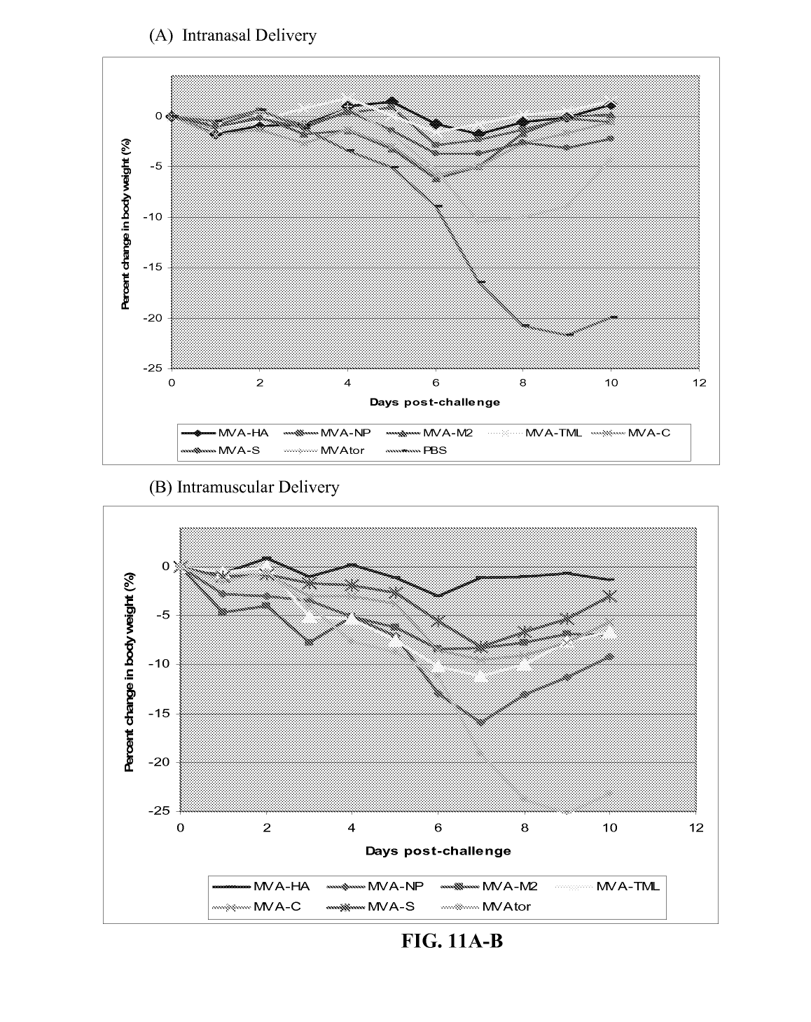

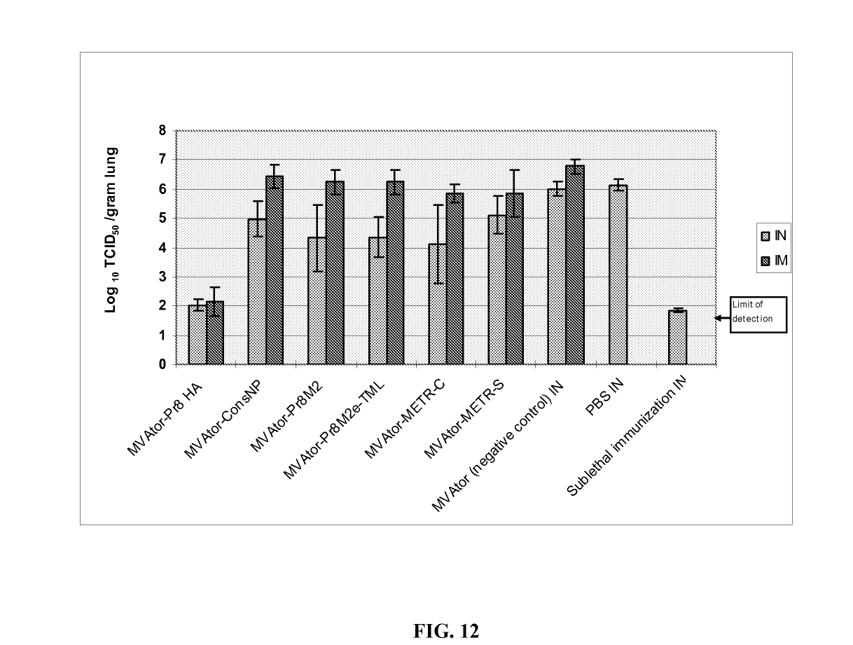

Viral burden in mice immunized intranasally (IN) or intramuscularly (IM) were measured. Viral load in lung tissues of mice immunized twice with the MVA vaccines were measured three days after challenge with influenza A virus. Data are shown in FIG. 12.

### Example 8

### Immune Response Against Low-Dose H1N1 PR8 Challenge in NP and M2 Immunized Mice

The efficacy of NP+M2 vaccination against homologous low dose influenza infection (H1N1 Pr8) was tested in mice. Mice for this study were immunized intramuscularly at days 0 and 21 with MVAtor-based vaccines containing conserved influenza antigens as summarized in Table 8.

TABLE 8
Treatment Groups

| | Animals | | |
|---|---|---|---|
| | | Viral Load | |
| Group | Body Weight | (per time point) | Immunization Material |
| 1 | 10 | 8 | PR8 HA |
| 2 | 10 | 8 | NP |
| 3 | 10 | 8 | M2 |
| 4 | 10 | 8 | NP + M2 |
| 5 | 10 | 8 | MVAtor |
| 6 | 10 | 8 | Sublethal infection with PR8 |

The mice were challenged with H1N1 PR8 administered intranasally (<2 LD50) at day 42. Body weight, survival, and viral load (days 2 and 4) were analyzed.

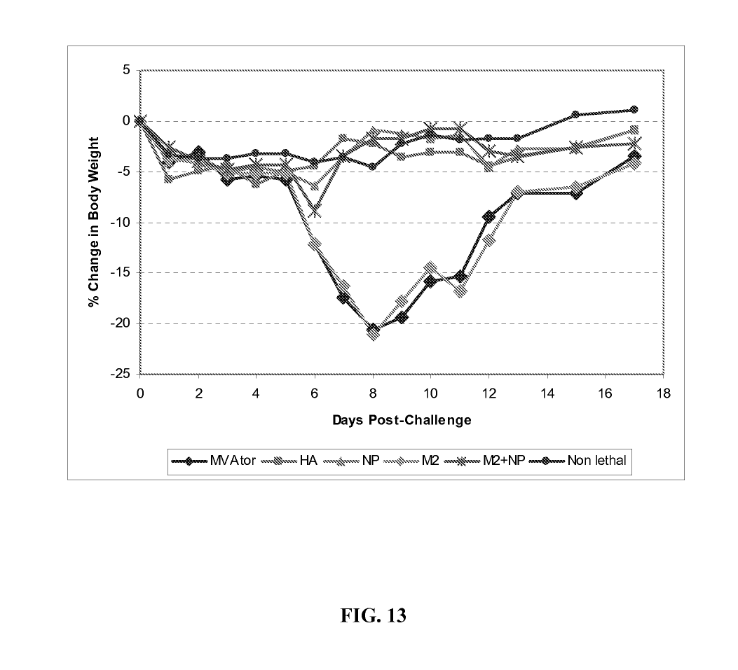

As shown in FIG. 13, the HA, NP, M2+NP, and nonlethal immunization groups were protected against body weight loss. In the M2 immunization group, 6 of 10 mice lost <20% body weight by day 8. In the MVAtor immunization group 10 of 10 mice lost <20% body weight by day 8. There was 100% survival for all treatment and control groups.

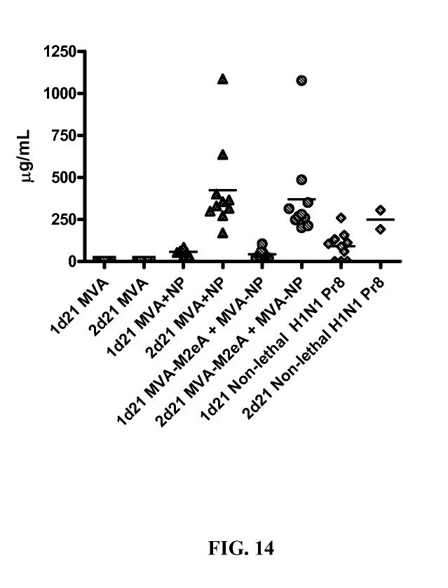

In a further study, the anti-NP immune response was tested by ELISA using recombinant NP (ImGenex) coated plates. The ELISA results are shown in FIG. 14 for 1d21 MVA, 2d21 MVA, 1d21 MVA+NP, 2d21 MVA+NP, 1d21 MVA-M2eA+MVA-NP, 2d21 MVA-M2eA+MVA-NP, 1d21 Non-lethal H1N1 PRS, and 2d21 Non-lethal H1N1 PR8. “1d21” refers to the immune response measured after a single vaccination, and “2d21” refers to the immune response measured after two doses of the vaccine construct. These results show that anti-NP immune responses were observed after two immunization.

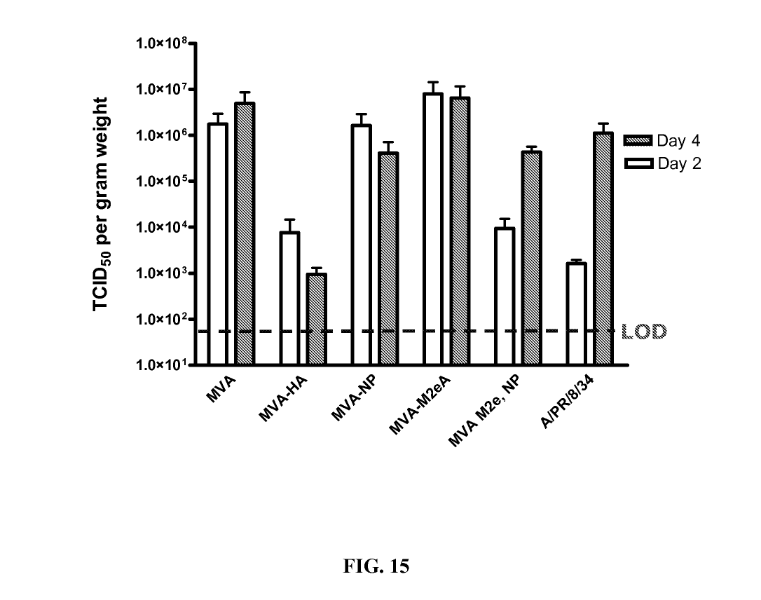

Viral load in lung tissues of mice immunized intramuscularly (IM) with MVA, MVA-HA, MVA-NP, MVA-M2eA, MVA-M2e+NP, or A/PR/8/34 was measured at days 2 and 4 after challenge with H1N1 PR8 virus. The viral load results are shown in FIG. 15. Animals immunized with MVA-HA had a 2-3 log reduction in lung load on both day 2 and day 4. The results show that animals immunized with NP+M2 showed 2-log reduction in lung vial load on day 2. However, there was no significant reduction in virus replication in the lungs on day 4 in any of the groups receiving NP or M2 antigens.

### Example 9

### Immune Response Against Lethal Dose H1N1 PR8 Challenge in NP and M2 Immunized Mice

The efficacy of NP+M2 vaccination against homologous lethal dose influenza infection (H1N1 Pr8) was tested in mice. Mice for this study were immunized intranasally (IN) or intramuscularly (IM) at days 0 and 21 with MVAtor-based vaccines containing conserved influenza antigens as summarized in Table 9.

TABLE 9
Treatment Groups

| | Animals | | |
|---|---|---|---|
| | | Viral Load | |
| Group | Body Weight | (per time point) | Immunization Material |
| 1 | 10 | 4 | PR8 HA |
| 2 | 10 | 4 | NP |
| 3 | 10 | 4 | M2 |
| 4 | 10 | 4 | M2-TML |
| 5 | 10 | 4 | METR-C |
| 6 | 10 | 4 | METR-S |
| 7 | 10 | 4 | NP + M2 |
| 8 | 10 | 4 | NP + M2 TML |
| 9 | 10 | 4 | NP + METR-C |
| 10 | 10 | 4 | NP + METR-S |
| 11 | 10 | 4 | MVAtor |
| 12 | 10 | 4 | PBS |

The mice were challenged with H1N1 PR8 administered intranasally (3 LD50) at day 42. Hemagglutination inhibition (HAI) (pre-challenge), body weight, survival, and viral load (day 3) were analyzed.

Influenza A H1N1 Puerto Rico 8/1934, abbreviated “PR8,” is a mouse-adapted influenza virus and was used in the HAI assay for this study. Prior to HAI, PR8 was assayed for Haemagglutinin capability using turkey red blood cells, and thereafter diluted to the appropriate concentration of 8 haemagglutinin units (dilution factor 1024) per mL in order to perform HAL For HAL 25 mcL of mouse serum was incubated with 75 mcL receptor destroying enzyme (RDE) for 30 minutes at 37° C. followed by dilution to 250 mcL (1/10 dilution of serum) in normal saline and storage at 4° C. until used.

Sera were tested using the HAI procedure in pools of 3 or 4 sera. Sera (25 mcL) were diluted in sequential 2-fold dilutions in 25 mcL PBS, to which 25 mcL of diluted influenza virus were added and incubated for 30 minutes at room temperature. Turkey red blood cells (50 mcL, washed and diluted) were added and incubated for 60 minutes at room temperature, followed by assessment for inhibition of haemaglutination. The HAI results are shown in Table 10.

TABLE 10
HAI Results

| | Animals immunized with | |
|---|---|---|
| | MVAtor vector indicating | Pre-challenge |
| Groups | antigens | (GMT) HAI |
| 1 | Sublethal dose-H1N1 | 119 ± 528 |
| 2 | MVA-HA | 1092 ± 740 |
| 11 | PBS | <20 |
| 13 | MVAtor-alone | <20 |

N = 8 mice/group;
<20 = below limit of detection

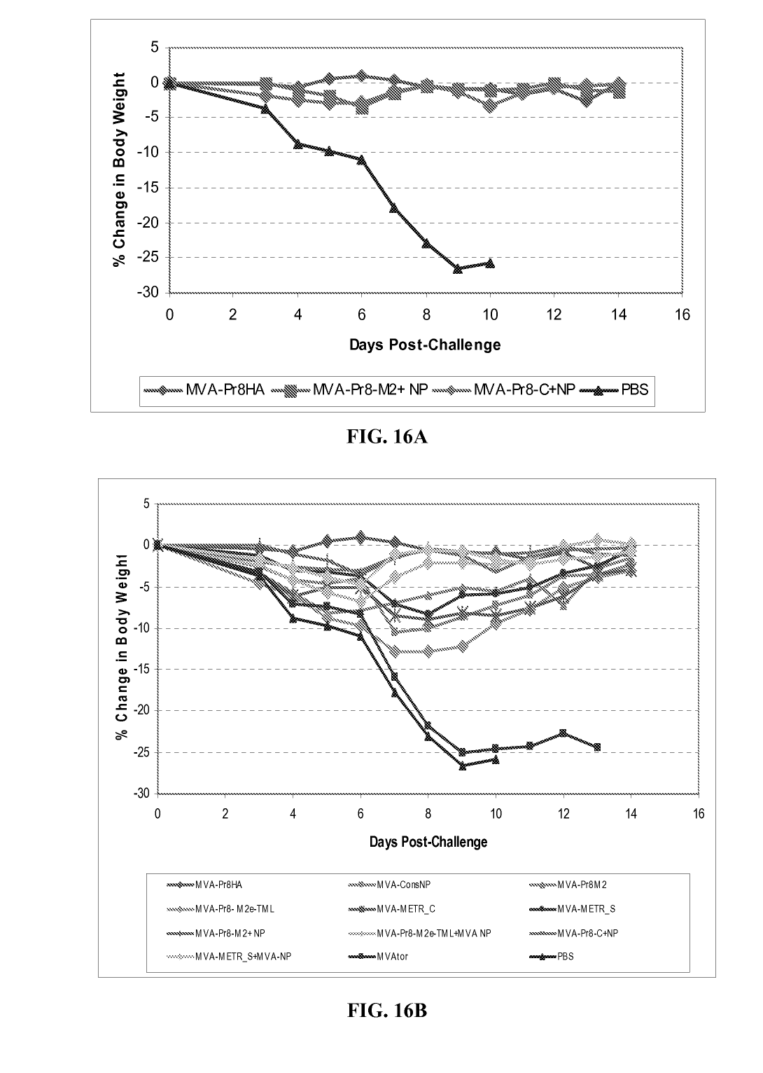

As shown in FIGS. 16A and 16B, the NP+M2 immunization groups were protected against body weight loss. All immunized groups were 100% protected against death, while MVAtor and PBS control groups resulted in 0% (0/10) survival.

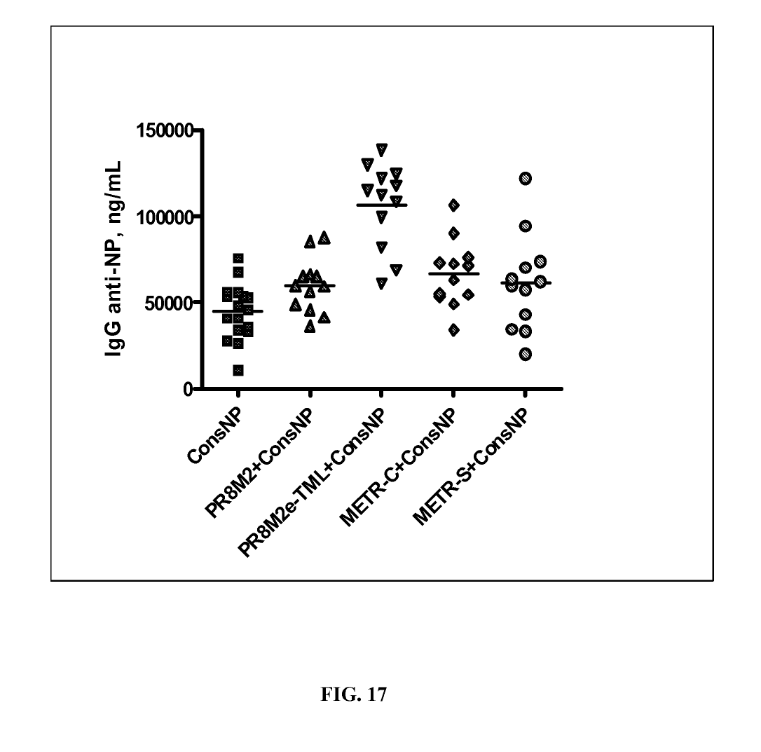

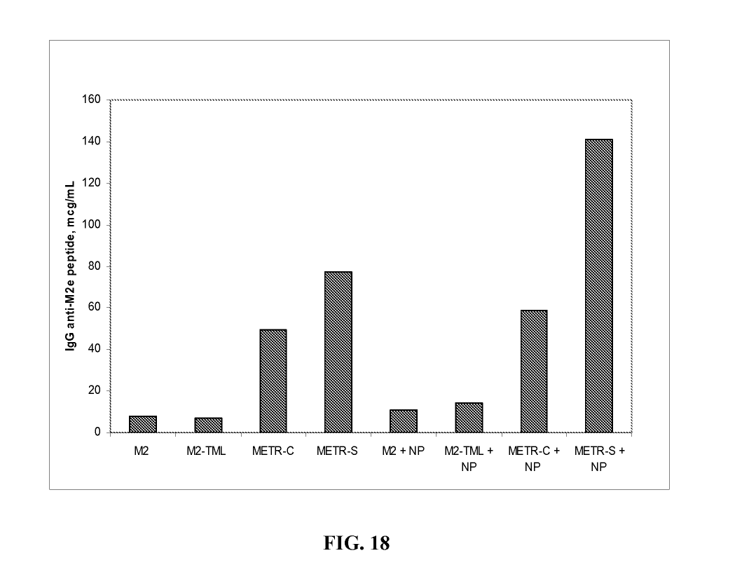

In a further study, the anti-NP and anti-M2 immune responses were tested by ELISA using recombinant NP (ImGenex) and M2 peptide #4 (SEQ ID NO: 4) coated plates, respectively. The anti-NP ELISA results are shown in FIG. 17 for ConsNP, Pr8M2+ConsNP, Pr8M2e-TML+ConsNP, METR-C+ConsNP, and METR-S+ConsNP. The anti-M2 ELISA results are shown in FIG. 18 for M2, M2-TML, METR-C, METR-S, M2+NP, M2-TML+NP, METR-C+NP, and METR-S+NP. These results show that anti-NP and anti-M2 immune responses were generated in vaccinated animals.

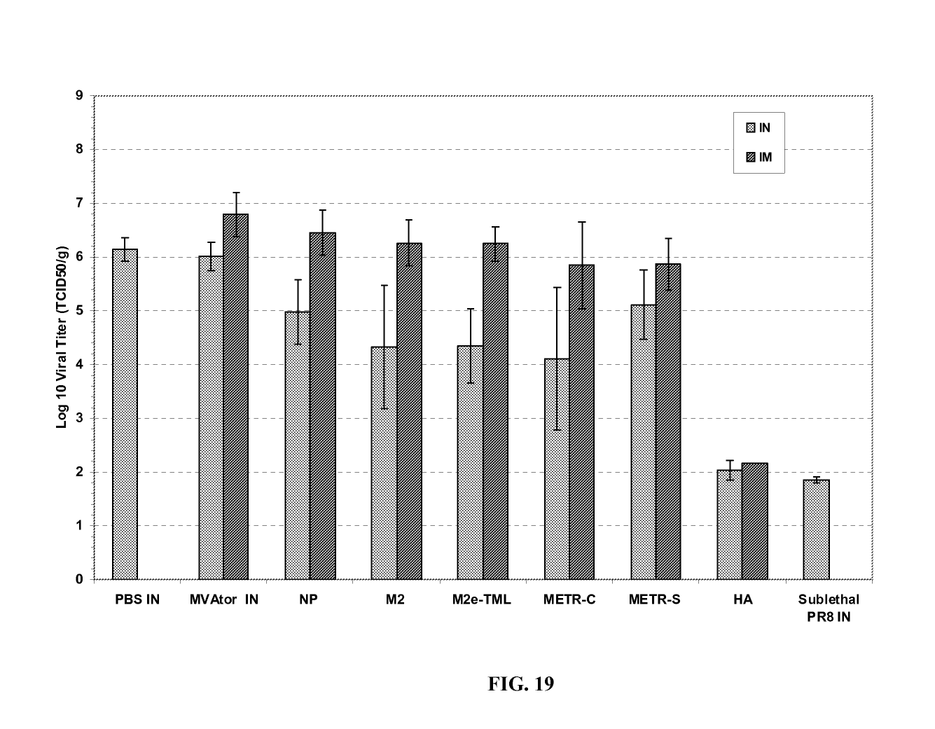

Viral load in lung tissues of mice immunized intranasally (IN) or intramuscularly (IM) with PBS IN, MVAtor, NP, M2, M2e-TML, METR-C, METR-S, HA, and sublethal PR8 IN was measured at day 3 after challenge with H1N1 PR8 virus. The viral load results are shown in FIG. 19. These results show that animals immunized intranasally with NP, M2, M2e-TML, METR-C and METR-S were partially protected against virus replication in the lungs. There was no significant reduction in virus replication in the lungs in the groups receiving intramuscular NP or M2 antigens.

### Example 10

### Immune Response Against Lethal Dose H1N1 Swine Challenge in NP, M2, and M1 Immunized Mice

The efficacy of NP+M2+M1 vaccination against homologous lethal dose influenza infection (sH1N1 A/Mx/4108/09) was tested in mice. Mice for this study were immunized intramuscularly at days 0 and 21 with MVAtor-based vaccines containing conserved influenza antigens as summarized in Table 11.

TABLE 11
Treatment Groups

| | Animals | | |
|---|---|---|---|
| | | Viral Load | |
| Group | Body Weight | (per time point) | Immunization Material |
| 1 | 10 | 5 | Flulaval (2010 TIV) |
| 2 | 10 | 5 | NP + METR-C + M1 |
| 3 | 10 | 5 | NP + M2 + M1 |
| 4 | 10 | 5 | NP + M2 |
| 5 | 10 | 5 | M1 |
| 6 | 10 | 5 | NP + M1 |
| 7 | 10 | 5 | MVAtor |
| 8 | 10 | 5 | PBS |

The mice were challenged with sH1N1 A/Mx/4108/09 administered intranasally (˜20 LD50) at day 42. Hemagglutination inhibition (HAT) (pre-challenge), body weight, survival, and viral load (days 2 and 4) were analyzed.

The HAI assay was performed as described above in Example 9. The HAI results are shown in Table 12.

TABLE 12
HAI Results

| Groups - | California/07/09 | Mexico/4108/09 |
|---|---|---|
| Flulaval | (pandemic H1N1) | (pandemic H1N1) |
| Animal # | Day 39 | Day 39 |
| 2081 | 160 | 80 |
| 2082 | 160 | 80 |
| 2083 | 160 | 80 |
| 2084 | 80 | 40 |
| 2085 | 80 | 40 |
| 2086 | 40 | 20 |
| 2087 | 160 | 80 |
| 2088 | 80 | 40 |
| 2029 | 80 | 40 |
| 2090 | 80 | 80 |
| 2091 | 160 | 40 |
| 2092 | 320 | 80 |
| 2093 | 160 | 80 |
| 2094 | 80 | 40 |
| 2095 | 80 | 40 |
| 2201 | 160 | 80 |
| 2202 | 80 | 40 |
| 2203 | 160 | 40 |
| Geomean | 113 (±65) | 52 (±23) |
| (±SD) | | |
| | CDC Ferret Reference | CDC Ferret Reference |
| | (A/Ca/04/09): 6400 | (A/Ca/04/09): 1600 |

Flulaval dose was given to mice: 150 μL, IM (4.5 μg HA of each of the 3 viruses); human dose: 500 μL, IM (15 μg HA of each of the 3 viruses). All pre-bleed samples for Group 1 (Flulaval) and Group 8 (PBS) were below limit of detection (<20). All day 39 (pre-challenge) samples for Group 8 (PBS) were below limit of detection for both H1N1 pandemic viruses.

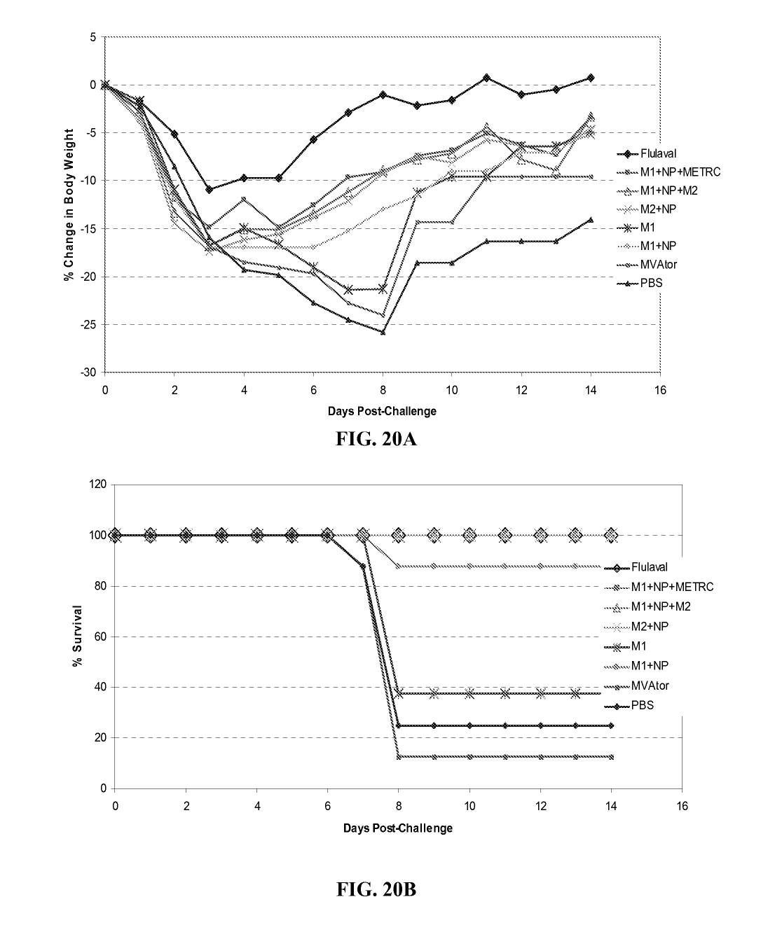

As shown in FIG. 20A, NP+M2 immunization did protect against body weight loss. However, a significant additional benefit was not observed when M1 was added to NP+M2. FIG. 20B shows the survival outcome for vaccinated mice. The survival results for this study are shown in Table 13 below.

TABLE 13
Survival Results

| Immunization Group | % Survival |
|---|---|
| Flulaval | 100% (8/8) |
| M1 + NP + METRC | 100% (8/8) |
| M1 + NP + M2 | 100% (8/8) |
| M2 + NP | 100% (8/8) |
| M1 | 38% (3/8) |
| M1 + NP | 88% (7/8) |
| MVAtor | 13% (1/8) |
| PBS | 25% (2/8) |

These results show that the mortality in groups that received M1+NP+M2 and M1+NP+METR-C was significantly (p=0.0035) lower than the PBS group.

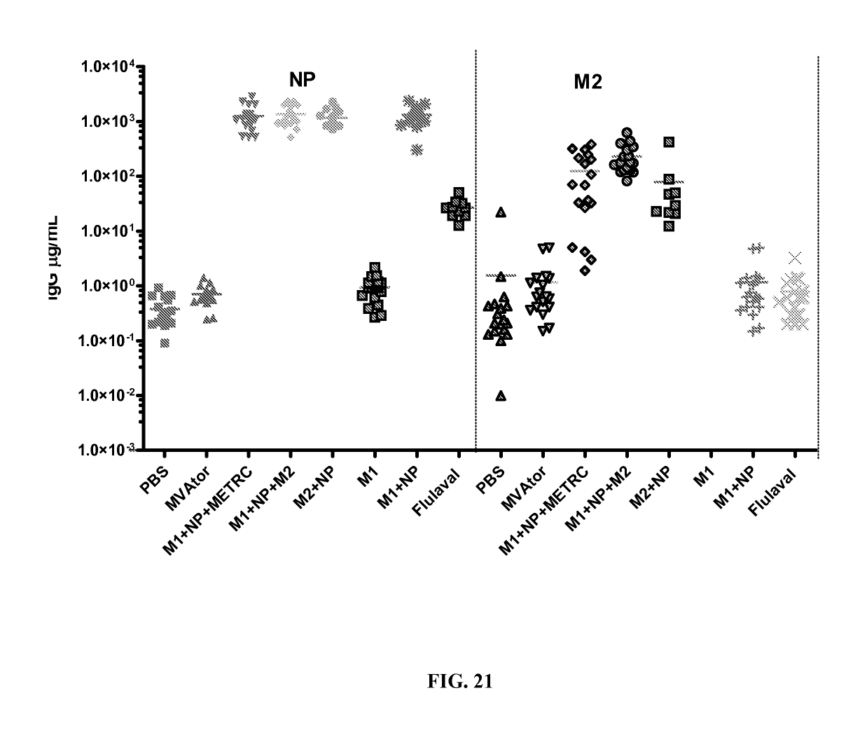

In a further study, the anti-NP and anti-M2 immune responses were tested by ELISA using recombinant NP (EPDU) and M2 peptide #4 (SEQ ID NO: 4) coated plates, respectively. The anti-NP and anti-M2 ELISA results are shown in FIG. 21 for PBS, MVAtor, M1+NP+METRC, M1+NP+M2, M2+NP, M1, M1+NP, and Flulaval. These results show that vaccination induced strong anti-NP and anti-M2 immune responses.

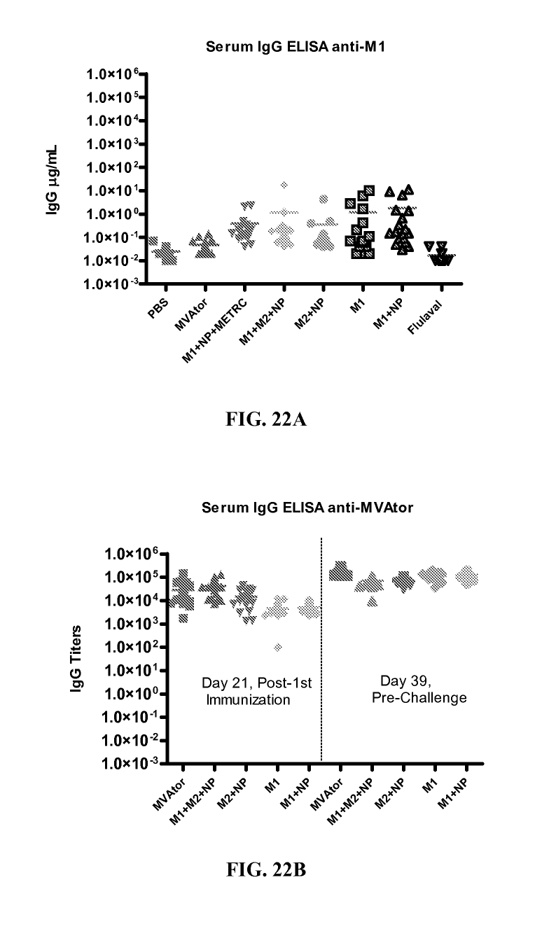

The anti-M1 and anti-MVA immune responses were tested by ELISA using recombinant M1 and MVA CT84 coated plates, respectively. The anti-M1 and anti-MVA ELISA results are shown in FIG. 22A-B for PBS, MVAtor, M1+NP+METRC, M1+M2+NP, M2+NP, M1, M1+NP, and Flulaval. These results show that vaccination did not induce an anti-M1 immune responses.

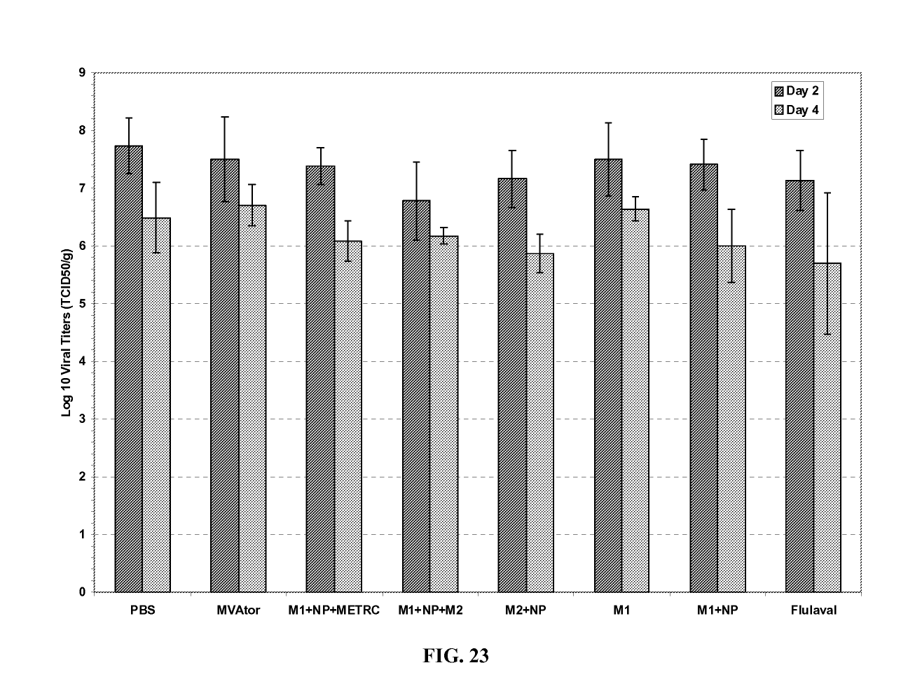

Viral load in lung tissues of mice immunized intramuscularly (IM) with PBS, MVAtor, M1+NP+METRC, M1+NP+M2, M2+NP, M1, M1+NP, and Flulaval was measured at days 2 and 4 after challenge with sH1N1 A/Mx/4108/09 virus. The viral replication in the lung results are shown in FIG. 23. The results show that the tested vaccines did not reduce virus replication in the lungs.

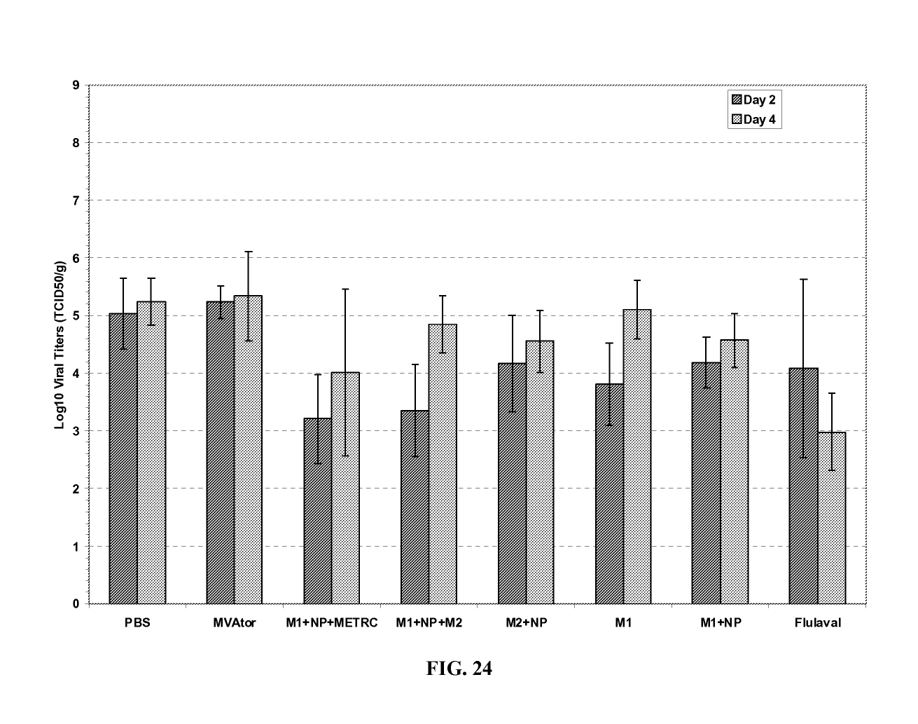

Viral load in nasal turbinates of mice immunized intramuscularly (IM) with PBS, MVAtor, M1+NP+METRC, M1+NP+M2, M2+NP, M1, M1+NP, and Flulaval was measured at days 2 and 4 after challenge with sH1N1 A/Mx/4108/09 virus. The viral replication in the lung results are shown in FIG. 24. The results show a partial reduction in viral load in nasal turbinates in all vaccinated groups. The addition of M1 to the vaccine did not increase the reduction in viral load in nasal turbinates.

1. An isolated polynucleotide comprising a coding region encoding a polypeptide, wherein said polypeptide comprises at least five influenza virus Matrix 2 protein (M2) ectodomain peptides.

2. (canceled)

3. An isolated polynucleotide comprising a coding region encoding a polypeptide, wherein said polypeptide comprises any three, four, five, six or more of the following M2 ectodomain peptides arranged in any order relative to each other:
ii. SEQ ID NO: 1 (M2e#1_C);
ii. SEQ ID NO: 2 (M2e#2_C);
iii. SEQ ID NO: 3 (M2e#3_C);
iv. SEQ ID NO: 4 (M2e#4_C);
v. SEQ ID NO: 5 (M2e#5_C);
vi. SEQ ID NO: 6 (M2e#6_C);
vii. SEQ ID NO: 7 (M2e#1_S);
viii. SEQ ID NO: 8 (M2e#2_S);
ix. SEQ ID NO: 9 (M2e#3_S);
x. SEQ ID NO: 10 (M2e#4_S);
xi. SEQ ID NO: 11 (M2e#5_S); and
xii, SEQ ID NO: 12 (M2e#6_S).

4. The polynucleotide of claim 3, wherein said polypeptide comprises any three or more of the following M2 ectodomain peptides arranged in any order relative to each ether:
i. SEQ ID NO: 1 (M2e#1_C);
ii. SEQ ID NO: 2 (M2e#2_C);
iii. SEQ ID NO: 3 (M2e#3_C);
iv. SEQ ID NO: 4 (M2e#4_C);
v. SEQ ID NO: 5 (M2e#5_C); and
vi. SEQ ID NO: 6 (M2e#6_C).

5. The polynucleotide of claim 3, said polypeptide comprises any three or more of the following M2 ectodomain peptides arranged in any order relative to each other:
i. SEQ ID NO: 7 (M2e#1_S);
ii. SEQ ID NO: 8 (M2e#2_S);
iii. SEQ ID NO: 9 (M2e#3_S);
iv. SEQ ID NO: 10 (M2e#4_S);
v. SEQ ID NO: 11 (M2e#5_S); and
vi. SEQ ID NO: 12 (M2e#6_S).

6-8. (canceled)

9. The polynucleotide of claim 1, wherein said polypeptide comprises the following M2 ectodomain peptides arranged in any order relative to each other: SEQ ID NO: 1 (M2e#1_C), SEQ ID NO: 2 (M2e#2_C), SEQ ID NO: 3 (M2e#3_C), SEQ ID NO: 4 (M2e#4_C), SEQ ID NO: 5 (M2e#5_C), and SEQ ID NO: 6 (M2e#6_C).

10. The polynucleotide of claim 1, wherein said polypeptide comprises the following M2 ectodomain peptides arranged in any order relative to each other: SEQ ID NO: 7 (M2e#1_S), SEQ ID NO: 8 (M2e#2_S), SEQ ID NO: 9 (M2e#3_S), SEQ ID NO: 10 (M2e#4_S), SEQ ID NO: 11 (M2e#5_S), and SEQ ID NO: 12 (M2e#6_S).

11. (canceled)

12. The polynucleotide of claim 1, wherein at least two of said Matrix 2 protein (M2) ectodomain peptides are fused together without a linker peptide interposed therebetween or with a linker peptide interposed therebetween.

13-17. (canceled)

18. The polynucleotide of claim 1, wherein said polypeptide further comprises at least one T cell epitope.

19. The polynucleotide of claim 18, wherein said at least one T cell epitope comprises one or more of SEQ ID NO: 26 and SEQ ID NO: 27.

20. The polynucleotide of claim 1, wherein said polypeptide comprises the amino acid sequence of SEQ ID NO: 16 or SEQ ID NO: 18.

21. (canceled)

22. The polynucleotide of claim 1, wherein said coding region is codon-optimized for expression in a human.

23. The polynucleotide of claim 1, wherein said coding region further comprises a promoter operably associated with said influenza virus Matrix 2 protein (M2) ectodomain peptide coding regions.

24. (canceled)

25. The polynucleotide of claim 1, wherein said coding region further comprises an additional polypeptide.

26. The polynucleotide of claim 25, wherein said additional polypeptide is hemagglutinin (HA), neuraminidase (NA), nucleoprotein (NP), Matrix 1 protein (M1), Matrix 2 protein (M2), non-structural protein (NS), RNA polymerase PA subunit (PA), RNA polymerase PB1 subunit (PB 1), RNA polymerase PB2 subunit (PB2), or a combination of two or more of said influenza polypeptides or fragments thereof.

27-31. (canceled)

32. A vector comprising the polynucleotide of claim 1.

33. (canceled)

34. The vector of claim 32, wherein said vector is selected from the group consisting of a vaccinia virus vector, an adeno virus vector, an adeno-associated virus vector, and a retroviral vector.

35. The vector of claim 34, wherein said vaccinia virus vector comprises a modified vaccinia virus Ankara (MVA).

36. The vector of claim 35, which further expresses an additional polypeptide.

37. The vector of claim 36, wherein said additional polypeptide is hemagglutinin (HA), neuraminidase (NA), nucleoprotein (NP), Matrix I protein (M1), Matrix 2 protein (M2), non-structural protein (NS), RNA polymerase PA subunit (PA), RNA polymerase PB 1 subunit (PB 1), RNA polymerase PB2 subunit (PB2), or a combination of two or more of said influenza polypeptides or fragments thereof.

38-41. (canceled)

42. The vector of claim 35, wherein said MVA is capable of replicating in an avian cell.

43-45. (canceled)

46. The vector of claim 42, wherein said avian cell is AGE1cr, AGE.pIX, or E866.

47. The vector of claim 35, wherein the polynucleotide is inserted in the MVA genome within a naturally-occurring deletion site.

48. (canceled)

49. The vector of claim 35, wherein the polynucleotide is inserted in an open reading frame (ORF) of the MVA genome.

50-52. (canceled)

53. A host cell comprising the polynucleotide of claim 1.

54-59. (canceled)

60. A polypeptide encoded by the polynucleotide of claim 1.

61. A method of producing an influenza polypeptide, comprising culturing the host cell of claim 53, and recovering said polypeptide.

62. A composition comprising the vector of claim 32, and a pharmaceutically acceptable carrier.

63. The composition of claim 62, further comprising an adjuvant.

64-70. (canceled)

71. A kit comprising: (a) the vector of claim 32; and (b) a means for administering said vector.

72. A method of inducing an immune response against an influenza virus in a subject in need thereof comprising administering to said subject an effective amount of the vector of claim 32.

73. The method of claim 72, wherein said immune response comprises an antibody response, a cell-mediated immune response, or a combination of an antibody response and a cell-mediated immune response.

74-76. (canceled)

77. A method for treating, preventing, or reducing the symptoms of an influenza virus infection or a condition associated with an influenza virus infection in a subject in need thereof comprising administering to said subject an effective amount of the vector of claim 32.

78. (canceled)

79. A method of vaccinating a subject in need thereof against an influenza virus infection comprising administering to said subject an effective amount of the vector of claim 32.

80-87. (canceled)

88. The method of claim 72, wherein the administering is performed via intradural injection, subcutaneous injection, intravenous injection, oral administration, mucosal administration, intranasal administration, or pulmonary administration.

89. (canceled)

90. The method of claim 72, wherein the method further comprises the step of administering at least one priming immunization.

91-92. (canceled)

93. The method of claim 72, wherein the method further comprises the step of administering at least one booster immunization.

94-98. (canceled)

99. A host cell comprising the vector of claim 35.

100. A method of producing a vaccine against an influenza virus comprising: (a) culturing the host cell of claim 99 and (b) isolating the MVA from said host cell.
